(19) United States
(12) Patent Application Publication
(10) Pub. No.: US 2024/0150333 A1
(43) Pub. Date: May 9, 2024

(54) TAF1 INHIBITORS

(71) Applicant: H. LEE MOFFITT CANCER CENTER AND RESEARCH INSTITUTE, INC., Tampa, FL (US)

(72) Inventors: Ernst SCHONBRUNN, Tampa, FL (US); Justin LOPCHUK, Tampa, FL (US); Md Rezaul KARIM, Tampa, FL (US); Zachary SHULTZ, Lakeland, FL (US)

(21) Appl. No.: 17/913,033

(22) PCT Filed: Mar. 22, 2021
(86) PCT No.: PCT/US21/23419

§ 371 Date: Sep. 20, 2022

Related U.S. Application Data
(60) Provisional application No. 62/992,475, filed on Mar. 20, 2020.

(51) Int. Cl. C07D 413/04; C07D 413/14; C07D 471/04

(52) U.S. Cl. CPC C07D 413/04; C07D 413/14; C07D 471/04

(57) ABSTRACT

Disclosed are inhibitors for TAF1. The TAF1 inhibitors are compounds having a structure according to one of the Formula described herein. Methods of using the disclosed compounds to treat proliferative disorders are also disclosed.

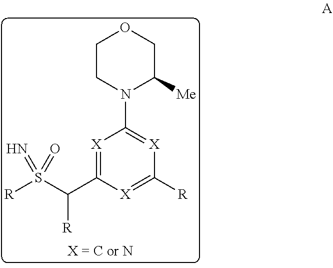

### BACKGROUND

Bromodomains (BRD) are highly conserved epigenetic “reader” modules that specifically recognize N-acetylated lysine (KAc) residues on histones and other proteins. Bromodomain-containing proteins control numerous functions including gene transcription and chromatin remodeling, gene splicing, protein scaffolding and signal transduction, and therefore, play fundamental roles in cell proliferation and division. A number of BRD-containing proteins, particularly those of the bromodomain and extra-terminal (BET) family, have been linked to tumorigenesis and inflammatory diseases. The landmark discoveries of potent small molecule inhibitors of BET bromodomains provided chemical tools to explore the function of proteins such as BRD4 in disease states for the first time. Since then several BRD4 inhibitors have entered clinical trials for oncology and cardiovascular indications. More recently, inhibitors targeting non-BET bromodomains, for which the physiological functions are not yet well understood, have been the subject of intense efforts in academia and pharmaceutical industry alike. Such inhibitors are valuable probes to unravel the function of bromodomains outside the BET family, their relevance in cancer and their potential as drug targets.

The bromodomain-containing protein TAF1 (TATA-box binding protein associated factor 1) is a subunit of the core promoter recognizing complex TFIID involved in general transcription. It is composed of several domains, a TBP (TATA binding protein) binding domain, an N-terminal kinase domain, a domain of unidentified function (DUF3591), a histone acetyltransferase domain, a winged-helix domain, a zinc knuckle motif, a tandem bromodomain (BD1 and BD2), a serine rich acidic tail domain, and a C-terminal kinase domain (FIG. 1). Recently, genomic landscape studies identified TAF1 as a significantly mutated gene in uterine serous carcinoma, and TAF1 overexpression has been described as a major factor for the high mitotic activity of human lung and breast carcinoma cells. TAF1 has been reported to inactivate p53 through phosphorylation at Thr55, translocating p53 to the cytoplasm and Mdm2-mediated degradation to induce G1/S-transition.24-26 Upon DNA damage, diacetylated p53 (K373ac and K382ac) directly interacts with the bromodomains of TAF1 to initiate gene transcription. Inactivation of TAF1 has been associated with activation of DNA damage response, similar to that mediated by ATR (Ataxia telangiectasia and Rad3-related protein). Temperature-sensitive mutations in the putative HAT domain of TAF1 caused p53 activation and cell cycle arrest, hallmarks of an ATR-mediated DNA damage response.

Transcription initiation factor TFIID subunit 1 (TAF1) is the largest subunit of general transcription factor TFIID and initiates the assembly of pre-initiation complex (PIC) for transcription by recognizing the core promoter elements of target genes. Full-length TAF1 is composed of several domains, including a tandem bromodomain (BRD) module, which performs a wide range of regulatory functions in transcription. For example, TAF1 N-terminal binding domain binds to Myc oncoprotein and assists Myc-driven gene transcription whereas TAF1 BRDs can directly interact with diacetylated p53 (K373ac and K382ac) to initiate the transcription of p53 target genes. TAF1 has also been reported to activate Mdm2-mediated p53 degradation leading to G1/S cell cycle transition. Cell lines with defective TAF1 exhibit hallmarks of an ATR-mediated DNA damage response (DDR). TAF1 has been found to be significantly mutated in uterine serous carcinoma, and TAF1 overexpression has been described as a major factor for the high mitotic activity in solid tumors.

Moreover, the acetyl-lysine recognition property of TAF1 BRD has been implicated in AML1-ETO driven acute myeloid leukemia (AML). Although deregulation of gene transcription and evolving plasticity are the underlying causes of cancer and resistance to cancer therapeutics, TAF1 remains an underexplored drug target. Of the multiple domains of TAF1 only the histone acetyl transferase (HAT) and BRDs have been structurally determined at high resolution (<3 Å). The HAT domain does not share the common architecture found in other HAT family members and requires association with TAF7 (Transcription initiation factor TFIID subunit 7) as an essential structural component. This pseudo-HAT domain is considered less druggable, while the BRDs have been shown to be tractable by small molecule inhibitors. Among the few TAF1 inhibitors reported to date are the pan-BRD inhibitor bromosporine (BSP), the moderately selective inhibitor BAY299 and the highly selective inhibitor GNE-371. Cancer cell growth inhibitory activity of BAY299 and GNE-371 was weak or not reported, but antiproliferative synergy with the BET inhibitor JQ1 was demonstrated. Biological effects upon chemical inhibition of TAF1 have not been reported and no TAF1 inhibitor has reached the clinic yet.

Combined, the present knowledge suggests that TAF1 is a promising yet underexplored target for the development of small molecule inhibitors directed at the transcription machinery of cancer cells through an epigenetic mechanism of action. There is a need for compositions and methods for chemical inhibition of the bromodomain of TAF1 alone and in combination with ATR in cancer. The precise role of the tandem bromodomain of TAF1 in the upregulation of oncogenic or inactivation of apoptotic pathways is unclear, and the effect of chemical TAF1 inhibition on transcriptional activity has not been reported yet. Previously, it was shown that TAF1 regulates cyclin A and D1 gene expression, Rb phosphorylation, DNA damage response (DDR) pathways and p21/p27 expression. However, the contribution of bromodomains in these signaling pathways is not known. Inhibiting the DDR has become a therapeutic concept in cancer therapy. Resistance to genotoxic therapies has been associated with increased DDR signaling, and many cancers are dependent on the functional DDR pathways for survival.

What are needed are new, potent and selective inhibitors for TAF1 and methods for their use. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. The compounds can have a structure represented by Formula I.

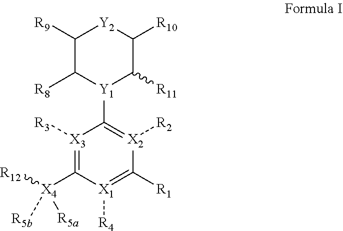

wherein
X₁, X₂, and X₃ are independently selected from C, N, or S;
X₄ is selected from C, N, or S;
Y₁ is selected from CH or N;
Y₂ is selected from O or NH;
R₁ is selected from halogen, amine, alkylamine, thiol, sulfide, sulfonyl, sulfoximine, sulfoxide, sulfur diimide, sulphonamide, alkoxyl, cycloalkyl, heterocycloalkyl, aryl, or heteroaryl, wherein R₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₂, R₃, and R₄, when present, are independently selected from, hydrogen, halogen, hydroxyl, cyano, carboxyl, C₁-C₃ alkyl, C₁-C₃ haloalkyl, or cyclopropyl; R₅ₐ and R_{5b}, when present, are independently selected from, hydrogen, halogen, hydroxyl, cyano, carboxyl, amino, aminoalkyl, C₁-C₆ alkyl, C₁-C₆ haloalkyl, cycloalkyl, heterocycloalkyl, or R₅ₐ and R_{5b} combine to form a carbonyl, a C₃-C₈ cycloalkyl or a C₂-C₇ heterocycloalkyl together with the atom to which they are attached, wherein R₅ₐ and R_{5b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₈ is selected from hydrogen, halogen, hydroxyl, cyano, carboxyl, amino, C₁-C₃ alkyl, C₁-C₃ haloalkyl, or C₁-C₃ aminoalkyl, wherein R₈ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₉ and R₁₀ are independently selected from hydrogen, halogen, hydroxyl, cyano, carboxyl, amino, C₁-C₃ alkyl, C₁-C₃ haloalkyl, or C₁-C₃ aminoalkyl, wherein R₉ and R₁₀ are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₁ is selected from C₁-C₃ alkyl, C₁-C₃ haloalkyl, or C₁-C₃ aminoalkyl, wherein R₁₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₂ is selected from sulfoximine, sulfonyl, sulfoxide, sulfur diimide, sulphonamide, amide, amine, hydroxyl, carbonyl, ester, alkyl, heteroalkyl, or one of R₅ₐ or R_{5b} combine with R₁₂ to form a C₃-C₈ cycloalkyl or a C₂-C₇ heterocycloalkyl together with the atom to which they are attached, wherein R₁₂ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl; and
----- represents a bond that is present or absent,
or a pharmaceutically acceptable salt thereof.

In some embodiments, the compound is not AZD6738, AZ20, or AZD3147 as described herein.

Pharmaceutical composition comprising a therapeutically effective amount of a compound described herein or a pharmaceutically acceptable salt thereof, and a pharmaceutically acceptable carrier are also disclosed.

In specific aspects, the disclosed subject matter relates to cancer therapy and to anti-cancer compounds. More specifically, the subject matter disclosed herein relates to inhibitors for the bromodomain-containing protein TAF1 (transcription initiation factor TFIID subunit 1). Further, the subject matter disclosed herein relates to inhibitors that are selective for TAF1. Also disclosed are methods of inhibiting the second bromodomain of TAF1. Methods of treating certain cancers are disclosed herein. In certain examples, the cancer is breast cancer or lung cancer.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

FIG. 1 show a schematic diagram of the domains structure of human TAF1. TBP BD, TATA binding protein binding domain; HAT, Histone acetyl transferase; DUF3591, Domain of unidentified functions 3591; BD1/2, Bromodomain 1 and 2; KD1/2, Kinase domain 1 and 2; WH, Wingedhelix DNA-binding motif and Zf, Zinc finger motif.

FIG. 2A-2E show discovery of dual TAF1-kinase inhibitors. (FIG. 2A) DSF thermal shift curves of the hit compounds identified from a kinase inhibitor library for TAF1-T (left) and TAF1-2 (right). Bromosporine (BSP) served as the positive control. (FIG. 2B) Chemical structures of the hits AZD6738 and BI2536, and AZ20, a close analog of AZD6738. (FIG. 2C) ITC thermograms of AZD6738 interaction with TAF1-2 (left panel) and TAF1-T (right panel). ITC conditions are shown in Table 3, thermodynamic parameters in FIG. 18. (FIG. 2D) MST signal response for AZD6738 interaction with TAF1-2. (FIG. 2E) Selectivity profile of AZD6738 across 32 BRDs (Bromoscan assay by Discoverx); values are listed in Table 4.

Selectivity and binding affinity of AZD6738 for TAF1. (FIG. 2A) Profiling of AZD6738 across 32 human bromodomains at 20 μM concentration (performed by DiscoveRx). (FIG. 2B) Direct binding studies of TAF1-T with inhibitors by ITC with Kd values indicated. The biphasic profile of BAY299 is due to the interactions with both BD1 (low affinity site) and BD2 (high affinity site).

FIGS. 3A-3D. Co-crystal structures of TAF1 with AZD6738 and analogues. (FIG. 3A) Chemical structures of AZD6738 and analogues. (FIG. 3B) Inhibitor bound to BD2 (surface colored according to electrostatic potential). (FIG. 3C) Binding pose of the inhibitors in the KAc site. The conserved Asn1583 is colored in magenta. (FIG. 3D) H-bonding potential of functional groups with KAc site residues. Note that the sulfonyl groups of AZ20 and AZD3147 are not capable of establishing a H-bond with Pro1531 (dotted lines). Also note the structural changes of Phe1536 and Tyr1589 are caused by loss of shape complementarity with AZD3147.

FIGS. 4A-4F show kinase inhibitors stabilize distinct conformational states of TAF1 tandem bromodomain. Crystal structures of TAF1-T were determined in unliganded state (FIG. 4A, PDB 7JJH) and liganded states with AZD6738 (FIG. 4B, PDB 7K03), AZ20 (FIG. 4C, PDB 7K27) and AZD3147 (FIG. 4D, PDB 7K0D). BD1, BD2, and the linker region are labeled. Inhibitors exclusively occupied the KAc site of BD2. (FIG. 4E) Inhibitor-induced structural transitions of TAF1-T with distances between the KAc sites of BD1 and BD2 indicated in A. The insets show the H-bonds formed between residues of the domain-domain interface. (FIG. 4F) Conformational states of the linker region in the unliganded and liganded structures.

FIGS. 5A-5D show inhibition of TAF1 activates p53 and induces DNA damage signaling. (FIG. 5A) A549 lung cancer cells were treated with indicated compounds (10 μM, 24 h) in the absence and presence of 5 μM Nutlin and analyzed for p53 pathway markers by western blot. (FIG. 5B) Same as FIG. 13A) with and without gamma radiation for 8 h. (FIG. 5C) HCT116 colon cancer cells with and without p53 were treated with increasing concentrations of BAY299 and gamma radiation. Samples were analyzed 8 hrs after irradiation by western blot. (FIG. 5D) HCT116 cells treated with 10 μM inhibitors for 20 hrs were analyzed by western blot for indicated markers.

FIGS. 6A-6C show inhibition of TAF1 cooperates with MDM2 inhibitor to induce apoptosis. (FIG. 6A) HCT116 cells with and without p53 were treated with BAY299 in the presence and absence of 5 μM Nutlin for 48 hrs. Cell survival was determined by MTS assay in 4 experiments (mean+/−SD). (FIG. 6B) Phase contrast images of A549 cells treated with Nutlin, BAY299 and combination for 24 hrs. Dead cells appeared rounded and detached. (FIG. 6C) HCT116 cells treated with BAY299 and Nutlin for 48 hrs were analyzed for PARP cleavage by We stem blot.

FIGS. 7A-7B. Comparison of the binding modes of AZD6738 in TAF1 and that of a close analogue in PI3Kα. The upper panel shows the H-bonding partners in the ATP and KAc sites. Orange and magenta indicate hinge and conserved asparagine residues, respectively. The bottom panel is a schematic drawing to indicate potential hydrophobic VDW interactions and exposure to solvent.

FIGS. 8A-8C. Structures of TAF1 inhibitors based on AZD6738. (FIG. 8A) Core structure with proposed modification sites indicated. (FIG. 8B) Diversity-oriented route to analogs with a pyrimidine core. (FIG. 8C) Diversity-oriented route to analogs with a phenyl core.

FIGS. 9A-9B show structural basis of kinase inhibitor binding in TAF1. (FIG. 9A) Co-crystal structure of TAF1-2 with AZD6738 (PDB 7JSP). Shown are KAc site residues comprising the binding site, canonical interaction of the methylmorpholine oxygen of the inhibitor with conserved residue N1583 (magenta) and additional H-bonding interactions of the sulfoximine moiety with P1531 and N1533. (FIG. 9B) Same as (FIG. 9A) for inhibitor AZ20 (PDB 7JJG), the sulfonyl moiety of which is less suited for interaction with P1531 (orange dotted line). VDW hydrophobic and H-bonding interactions are shown in FIG. 5, and electron density maps of the respective ligands in FIG. 19. Binding mode of AZD6738 in ATR mimicking mutant PI3Kα based on PDB 5UK8 compared with its binding mode in TAF1-2 (FIGS. 7A-7B). H-bonds are indicated as black dotted lines.

FIGS. 10A-10G show TAF1-T undergoes large conformational changes upon interaction with inhibitors in solution. TAF1-T in the absence and presence of AZD6738 and BAY299 was subjected to SEC-SAXS studies. (FIG. 10A) SEC elution profiles, (FIG. 10B) SAXS scattering profiles, (FIG. 10C) Pair distance distribution p(r) function. (FIG. 10D) Macromolecule particle parameters (Rg), maximum intraparticle distances (Dmax) and estimated molecular weights (volume of correlation, Vc and Porod's volume, Vp). (FIG. 10E) Ab initio envelopes (semi-transparent shape) and resulting models of TAF1-T upon rigid body refinement of BD1 and BD2. (FIG. 10F) ITC thermograms of TAF-T and individual bromodomains upon interaction with BAY299 and (FIG. 10G) resulting thermodynamic signatures.

FIGS. 11A-11C show binding potential of kinase inhibitors for TAF1 determined by independent assays. (FIG. 11A) DSF dose response of AZD6738 showing similar increase in thermostability for TAF1-2 and TAF1-T. (FIG. 11B) IC50 determination of AZD6738, AZ20 and bromosporine against TAF1-2 by AlphaScreen assay (Reaction Biology Corporation. (FIG. 11C) Kd determination of AZD6738 (left) and AZ20 (right) against TAF1-2 by BromoScan (DiscoverX). Outliers indicated in light colors were omitted for data fitting.

FIGS. 12A-12D show detailed binding interactions of inhibitors in the KAc site of TAF1 BD2. (FIG. 12A) AZD6738 (PDB 7JSP), (FIG. 12B) AZ20 (PDB 7JJG), (FIG. 12C) BI2536 (PDB 7KOU), (FIG. 12D) Bromosporine (PDB 7K1P).

FIG. 13 shows chemical structures of other compounds used in the examples.

FIGS. 14A-14C show detailed view on the interaction of kinase inhibitors in BD2 of TAF1-T. (FIG. 14A) AZD6738 (PDB 7K03), (FIG. 14B) AZ20 (PDB 7K27), (FIG. 14C) AZD3147 (PDB 7K0D).

FIG. 15 shows TAF1-T remains in an open state with MES bound in the KAc site of BD2. Superposition of unliganded TAF1-T (PDB 7JJH) and TAF1-T liganded with MES (PDB 7K6F). The inset shows the H-bonds between MES and KAc site residues and water molecules (spheres).

FIGS. 16A-16H show SAXS data evaluation for unliganded and liganded TAF1-T. Two-dimensional plots (integrated intensity on left Y axis and calculated particle parameters on the right Y axis) of SEC-SAXS frames for (FIG. 16A) TAF1-T alone, (FIG. 16B) with AZD6738 and (FIG. 16C) with BAY299. (FIG. 16D) SAXS profile (upper panel) and residual value plot (lower panel) of curve fitting for unliganded TAF1-T. (FIG. 16E) same as (FIG. 16D) for TAF1-T with AZD6738. (FIG. 16F) same as (FIG. 16D) for TAF1-T with BAY299. FIG. 16G) Normalized Kratky plot for the subjected experimental samples. FIG. 16H) Guinier analysis for radius of gyration (Rg) determination.

FIGS. 17A-17C show SAXS envelope construction of TAF1-T. (FIG. 17A) Reconstructed ab initio electron density maps generated from SAXS data by DENSS for unliganded TAF1-T (left), TAF1-T+AZD6738 (middle) and TAF1-T+BAY299 (right). (FIG. 17B) Ab initio bead model and averaged core of 20 bead models generated from SAXS data using DAMMIN for TAF1-T (left), TAF1-T+AZD6738 (middle) and TAF1-T+BAY299 (right). (FIG. 17C) Statistics of the ab initio electron density maps reconstructed from SAXS data using DENSS and the TAF1-T models fitted in the ab initio low-resolution envelopes using Crysol 3.0 program from the ATSAS suit. Resolution (A) of ab initio electron density map was calculated from Fourier shell correlation value of 0.5 (FSC0.5) of DENSS. χ² values, goodness of the fit, for the model fit were calculated by Crysol 3.0 program.

FIGS. 18A-18B show ITC data for TAF1 interaction with inhibitors. (FIG. 18A) Thermograms of bromosporine (BSP) interaction with TAF1-2 and TAF1-T. (FIG. 18B) Thermodynamic parameters of BSP and AZD6738 interaction with TAF1.

FIGS. 19A-19H show electron density maps of ligands bound in the KAc site of TAF1 BD2. The 2Fo-Fc density map upon refinement with ligand is shown contoured at IG. The Fo-Fc density map upon refinement omitting the ligand is shown contoured at 36. (FIGS. 19A-D) TAF1-2, (FIGS. 19E-H) TAF1-T.

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “reduce” or other forms of the word, such as “reducing” or “reduction,” is meant lowering of an event or characteristic (e.g., tumor growth, metastasis). It is understood that this is typically in relation to some standard or expected value, in other words it is relative, but that it is not always necessary for the standard or relative value to be referred to. For example, “reduces tumor growth” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as tumor growth or metastasis), diminishment of extent of cancer, stabilized (i.e., not worsening) state of cancer, preventing or delaying spread (e.g., metastasis) of the cancer, delaying occurrence or recurrence of cancer, delay or slowing of cancer progression, amelioration of the cancer state, and remission (whether partial or total).

The term “patient” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The symbols Aⁿ is used herein as merely a generic substituent in the definitions below.

The term “alkoxy” as used herein is an alkyl group bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O—.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN.

The term “azido” as used herein is represented by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

It is to be understood that the compounds provided herein may contain chiral centers. Such chiral centers may be of either the (R—) or (S—) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to administration of the compound in its (S—) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more doses. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

Reference will now be made in detail to specific aspects of the disclosed materials, compounds, compositions, articles, and methods, examples of which are illustrated in the accompanying Examples.

### Compounds

Disclosed herein are compounds having Formula I′.

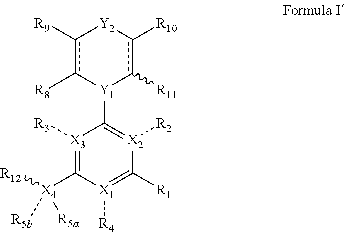

wherein
X₁, X₂, and X₃ are independently selected from C, N, or S;
X₄ is selected from C, N, or S;
Y₁ is selected from CH or N;
Y₂ is selected from O or NH;
R₁ is selected from halogen, amine, alkylamine, thiol, sulfide, sulfonyl, sulfoximine, sulfoxide, sulfur diimide, sulphonamide, alkoxyl, cycloalkyl, heterocycloalkyl, aryl, or heteroaryl, wherein R₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₂, R₃, and R₄, when present, are independently selected from, hydrogen, halogen, hydroxyl, cyano, carboxyl, C₁-C₃ alkyl, C₁-C₃ haloalkyl, or cyclopropyl;
R₅ₐ and R_{5b}, when present, are independently selected from, hydrogen, halogen, hydroxyl, cyano, carboxyl, amino, aminoalkyl, C₁-C₆ alkyl, C₁-C₆ haloalkyl, cycloalkyl, heterocycloalkyl, or R₅ₐ and R_{5b} combine to form a carbonyl, a C₃-C₈ cycloalkyl or a C₂-C₇ heterocycloalkyl together with the atom to which they are attached, wherein R₅ₐ and R_{5b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₈ is selected from hydrogen, halogen, hydroxyl, cyano, carboxyl, amino, C₁-C₃ alkyl, C₁-C₃ haloalkyl, or C₁-C₃ aminoalkyl, wherein R₈ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₉ and R₁₀ are independently selected from hydrogen, halogen, hydroxyl, cyano, carboxyl, amino, C₁-C₃ alkyl, C₁-C₃ haloalkyl, or C₁-C₃ aminoalkyl, wherein R₉ and R₁₀ are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₁ is selected from C₁-C₃ alkyl, C₁-C₃ haloalkyl, or C₁-C₃ aminoalkyl, wherein R₁₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₂ is selected from sulfoximine, sulfonyl, sulfoxide, sulfur diimide, sulphonamide, amide, amine, hydroxyl, carbonyl, ester, alkyl, heteroalkyl, or one of R₅ₐ or R_{5b} combine with R₁₂ to form a C₃-C₈ cycloalkyl or a C₂-C₇ heterocycloalkyl together with the atom to which they are attached, wherein R₁₂ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl; and
[symbol] represents a bond that is present or absent,
or a pharmaceutically acceptable salt thereof, and

wherein the compound is not AZD6738, AZ20, or AZD3147.

In certain aspects, disclosed herein are compounds having Formula I:

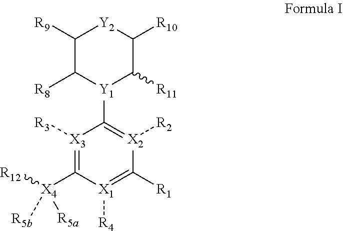

wherein
X₁, X₂, and X₃ are independently selected from C, N, or S;
X₄ is selected from C, N, or S;
Y₁ is selected from CH or N;
Y₂ is selected from O or NH;
R₁ is selected from halogen, amine, alkylamine, thiol, sulfide, sulfonyl, sulfoximine, sulfoxide, sulfur diimide, sulphonamide, alkoxyl, cycloalkyl, heterocycloalkyl, aryl, or heteroaryl, wherein R₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₂, R₃, and R₄, when present, are independently selected from, hydrogen, halogen, hydroxyl, cyano, carboxyl, C₁-C₃ alkyl, C₁-C₃ haloalkyl, or cyclopropyl;
R₅ₐ and R_{5b}, when present, are independently selected from, hydrogen, halogen, hydroxyl, cyano, carboxyl, amino, aminoalkyl, C₁-C₆ alkyl, C₁-C₆ haloalkyl, cycloalkyl, heterocycloalkyl, or R₅ₐ and R_{5b} combine to form a carbonyl, a C₃-C₈ cycloalkyl or a C₂-C₇ heterocycloalkyl together with the atom to which they are attached, wherein R₅ₐ and R_{5b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₈ is selected from hydrogen, halogen, hydroxyl, cyano, carboxyl, amino, C₁-C₃ alkyl, C₁-C₃ haloalkyl, or C₁-C₃ aminoalkyl, wherein R₈ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₉ and R₁₀ are independently selected from hydrogen, halogen, hydroxyl, cyano, carboxyl, amino, C₁-C₃ alkyl, C₁-C₃ haloalkyl, or C₁-C₃ aminoalkyl, wherein R₉ and R₁₀ are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₁ is selected from C₁-C₃ alkyl, C₁-C₃ haloalkyl, or C₁-C₃ aminoalkyl, wherein R₁₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₂ is selected from sulfoximine, sulfonyl, sulfoxide, sulfur diimide, sulphonamide, amide, amine, hydroxyl, carbonyl, ester, alkyl, heteroalkyl, or one of R₅ₐ or R_{5b} combine with R₁₂ to form a C₃-C₈ cycloalkyl or a C₂-C₇ heterocycloalkyl together with the atom to which they are attached, wherein R₁₂ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl; and
[symbol] represents a bond that is present or absent, or
or a pharmaceutically acceptable salt thereof.

Also disclosed are compounds having Formula II′.

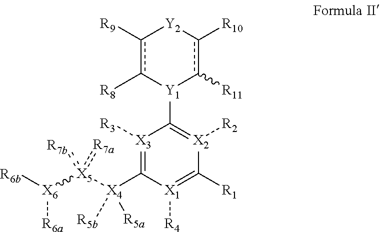

wherein
X₁, X₂, and X₃ are independently selected from C, N, or S;
X₄ is selected from C, N, or S;
X₅ is selected from O, C, N, or S;
X₆ is selected from O, C, or N;
Y₁ is selected from CH or N;
Y₂ is selected from O or NH;
R₁ is selected from halogen, amine, alkylamine, thiol, sulfide, sulfonyl, sulfoximine, sulfoxide, sulfur diimide, sulphonamide, alkoxyl, cycloalkyl, heterocycloalkyl, aryl or heteroaryl, wherein R₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₂, R₃, and R₄, when present, are independently selected from, hydrogen, halogen, hydroxyl, cyano, carboxyl, C₁-C₃ alkyl, C₁-C₃ haloalkyl, or cyclopropyl;
R₅ₐ and R_{5b}, when present, are independently selected from, hydrogen, halogen, hydroxyl, cyano, carboxyl, amino, aminoalkyl, C₁-C₆ alkyl, C₁-C₆ haloalkyl, cycloalkyl, heterocycloalkyl, or R₅ₐ and R_{5b} combine to form a carbonyl, a C₃-C₈ cycloalkyl or a C₂-C₇ heterocycloalkyl together with the atom to which they are attached, wherein R₅ₐ and R_{5b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₆ₐ and R_{6b} are independently selected from, hydrogen, halogen, hydroxyl, cyano, carboxyl, amino, aminoalkyl, C₁-C₆ alkyl, C₁-C₆ haloalkyl, cycloalkyl, heterocycloalkyl, aryl, heteroaryl, or R₆ₐ and R_{6b} combine to form a C₃-C₈ cycloalkyl, a C₂-C₇ heterocycloalkyl, an aryl, or a heteroaryl together with the atom to which they are attached, wherein R₆ₐ and R_{6b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl; or
wherein two or more of R₅ₐ, R_{5b}, R₆ₐ, and R_{6b} combine together with the atom to which they are attached to form a C₂-C₇ heterocycloalkyl or C₂-C₇ heterocycloalkenyl;
R₇ₐ and R_{7b} are independently absent or present and when present, are selected from O, hydroxyl, halogen, nitro, sulfonyl, thiol, sulfide, cyano, haloalkyl, or NH;
R₈ is selected from hydrogen, halogen, hydroxyl, cyano, carboxyl, amino, C₁-C₃ alkyl, C₁-C₃ haloalkyl, or C₁-C₃ aminoalkyl, wherein R₈ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₉ and R₁₀ are independently selected from hydrogen, halogen, hydroxyl, cyano, carboxyl, amino, C₁-C₃ alkyl, C₁-C₃ haloalkyl, or C₁-C₃ aminoalkyl, wherein R₉ and R₁₀ are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₁ is selected from C₁-C₃ alkyl, C₁-C₃ haloalkyl, or C₁-C₃ aminoalkyl, wherein R₁₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl; and
[symbol] represents a bond that is present or absent,
or a pharmaceutically acceptable salt thereof, and
wherein the compound is not AZD6738, AZ20, or AZD3147 as described herein.

In certain aspects, the compounds can have a structure as represented by Formula II:

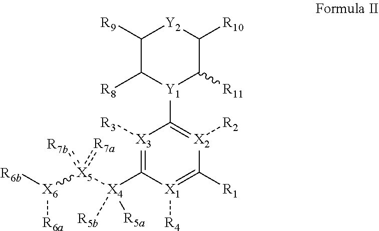

wherein
X₁, X₂, and X₃ are independently selected from C, N, or S;
X₄ is selected from C, C—OH, N, or S;
X₅ is selected from O, C, N, or S;
X₆ is selected from O, C, or N;
Y₁ is selected from CH or N;
Y₂ is selected from O or NH;
R₁ is selected from halogen, amine, alkylamine, thiol, sulfide, sulfonyl, sulfoximine, sulfoxide, sulfur diimide, sulphonamide, alkoxyl, cycloalkyl, heterocycloalkyl, aryl or heteroaryl, wherein R₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₂, R₃, and R₄, when present, are independently selected from, hydrogen, halogen, hydroxyl, cyano, carboxyl, C₁-C₃ alkyl, C₁-C₃ haloalkyl, or cyclopropyl;
R₅ₐ and R_{5b}, when present, are independently selected from, hydrogen, halogen, hydroxyl, cyano, carboxyl, amino, aminoalkyl, C₁-C₆ alkyl, C₁-C₆ haloalkyl, cycloalkyl, heterocycloalkyl, or R₅ₐ and R_{5b} combine to form a carbonyl, a C₃-C₈ cycloalkyl or a C₂-C₇ heterocycloalkyl together with the atom to which they are attached, wherein R₅ₐ and R_{5b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₆ₐ and R_{6b} are independently selected from, hydrogen, halogen, hydroxyl, cyano, carboxyl, amino, aminoalkyl, C₁-C₆ alkyl, C₁-C₆ haloalkyl, cycloalkyl, heterocycloalkyl, aryl, heteroaryl, or R₆ₐ and R_{6b} combine to form a C₃-C₈ cycloalkyl, a C₂-C₇ heterocycloalkyl, an aryl, or a heteroaryl together with the atom to which they are attached, wherein R₆ₐ and R_{6b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl; or
wherein two or more of R₅ₐ, R_{5b}, R₆ₐ, and R_{6b} combine together with the atom to which they are attached to form a C₂-C₇ heterocycloalkyl or C₂-C₇ heterocycloalkenyl;
R₇ₐ and R_{7b} are independently absent or present and when present, are selected from O or NH;
R₈ is selected from hydrogen, halogen, hydroxyl, cyano, carboxyl, amino, C₁-C₃ alkyl, C₁-C₃ haloalkyl, or C₁-C₃ aminoalkyl, wherein R₈ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₉ and R₁₀ are independently selected from hydrogen, halogen, hydroxyl, cyano, carboxyl, amino, C₁-C₃ alkyl, C₁-C₃ haloalkyl, or C₁-C₃ aminoalkyl, wherein R₉ and R₁₀ are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₁ is selected from C₁-C₃ alkyl, C₁-C₃ haloalkyl, or C₁-C₃ aminoalkyl, wherein R₁₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl; and
[symbol] represents a bond that is present or absent,
or a pharmaceutically acceptable salt thereof.

In certain aspects, the compounds can have a structure as represented by Formula II-A:

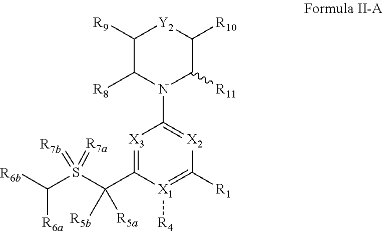

wherein
X₁, X₂, and X₃ are independently selected from C, CH, or N;
Y₂ is selected from O or NH;
R₁ is selected from heterocycloalkyl, aryl or heteroaryl, wherein R₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₄, when present, is selected from, hydrogen, halogen, hydroxyl, cyano, carboxyl, C₁-C₃ alkyl, C₁-C₃ haloalkyl, or cyclopropyl;
R₅ₐ and R_{5b} are independently selected from, C₁-C₃ alkyl, C₁-C₃ haloalkyl, C₃-C₈ cycloalkyl, C₁-C₃ heterocycloalkyl, or R₅ₐ and R_{5b} combine to form a C₃-C₈ cycloalkyl or a C₂-C₇ heterocycloalkyl together with the atom to which they are attached, wherein R₅ₐ and R_{5b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₆ₐ and R_{6b} are independently selected from, hydrogen, halogen, hydroxyl, cyano, amino, aminoalkyl, C₁-C₆ alkyl, C₁-C₆ haloalkyl, C₃-C₆ cycloalkyl, heterocycloalkyl, aryl, heteroaryl, or R₆ₐ and R_{6b} combine to form a C₃-C₈ cycloalkyl, a C₂-C₇ heterocycloalkyl, an aryl, or a heteroaryl together with the atom to which they are attached, wherein R₆ₐ and R_{6b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₇ₐ and R_{7b} are independently selected from O or NH;
R₈, R₉, and R₁₀ are independently selected from hydrogen, halogen, hydroxyl, cyano, amino, C₁-C₃ alkyl, C₁-C₃ haloalkyl, or C₁-C₃ aminoalkyl;
R₁₁ is selected from C₁-C₃ alkyl, C₁-C₃ haloalkyl, or C₁-C₃ aminoalkyl; and
[symbol] represents a bond that is present or absent,
or a pharmaceutically acceptable salt thereof.

In some embodiments of Formula I′, I, II′, II, or II-A, the compound is not AZD6738, AZ20, or AZD3147 as described herein.

Also disclosed are pharmaceutical compositions comprising a therapeutically effective amount of a disclosed compound, or a pharmaceutically acceptable salt thereof, and a pharmaceutically acceptable carrier.

In some aspects of Formula I′, I, II′, II, or II-A, R¹ can include a thiol, sulfide, sulfonyl, halogen, amine, alkoxyl, heterocycloalkyl, C₅-C₁₀ aryl, or C₂-C₉ heteroaryl, wherein R₁ is optionally substituted with alkyl, halogen, amine, hydroxyl, thiol, sulfide, or sulfonyl. In further aspects of Formula I′, I, II′, II, or II-A, R¹ can include a substituted aryl or a substituted or unsubstituted heteroaryl. For example, R¹ can include a 5-, 6- and 7-membered aromatic ring. The ring can be a carbocyclic, heterocyclic, fused carbocyclic, fused heterocyclic, bi-carbocyclic, or bi-heterocyclic ring system, which is optionally substituted as described herein. In some embodiments when R¹ is an heteroaryl, R¹ can include a monocyclic aromatic ring containing five or six ring atoms containing carbon and 1, 2, 3, or 4 heteroatoms each selected from non-peroxide oxygen, sulfur, and N(Y) where Y is absent or is H, O, (C₁-C₅) alkyl, phenyl or benzyl. Examples of aryl and heteroaryl rings include, but are not limited to, benzene, pyrrole, furan, thiophene, imidazole, oxazole, thiazole, triazole, pyrazole, pyridine, pyrazine, pyridazine and pyrimidine. The aromatic ring can be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, amino, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, and —CN.

In some embodiments, R¹ includes a polycyclic aryl or heteroaryl ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles, or both rings are aromatic. For example, in some embodiments when R¹ is a heteroaryl, R¹ can include an ortho-fused bicyclic heterocycle of about eight to ten ring atoms derived therefrom, particularly a benzene-derivative or one derived by fusing a propylene, trimethylene, or tetramethylene diradical thereto. Examples of heteroaryl include, but are not limited to, furyl, imidazolyl, triazolyl, triazinyl, oxazoyl, isoxazoyl, thiazolyl, isothiazoyl, pyraxolyl, pyrrolyl, pyrazinyl, tetrazolyl, pyridyl (or its N-oxide), thientyl, pyrimidinyl (or its N-oxide), indolyl, isoquinolyl (or its N-oxide), quinolyl (or its N-oxide), and the like.

In specific examples, R¹ is selected from a substituted C₅-C₆ aryl or a substituted or unsubstituted C₂-C₉ heteroaryl. For example, R¹ can be selected from a substituted phenyl, substituted or unsubstituted furyl, substituted or unsubstituted imidazolyl, substituted or unsubstituted triazolyl, substituted or unsubstituted triazinyl, substituted or unsubstituted oxazoyl, substituted or unsubstituted isoxazoyl, substituted or unsubstituted thiazolyl, substituted or unsubstituted isothiazoyl, substituted or unsubstituted pyrazolyl, substituted or unsubstituted pyrrolyl, substituted or unsubstituted pyrazinyl, substituted or unsubstituted tetrazolyl, substituted or unsubstituted pyridyl, substituted or unsubstituted thienyl, substituted or unsubstituted pyrimidinyl, substituted or unsubstituted indolyl, substituted or unsubstituted isoquinolyl, substituted or unsubstituted quinolyl, substituted or unsubstituted benzothienyl, substituted or unsubstituted benzofuranyl, substituted or unsubstituted benzoxazolyl, substituted or unsubstituted benzimidazolyl, substituted or unsubstituted benzothiazolyl, substituted or unsubstituted isoindolyl, substituted or unsubstituted indolinyl, substituted or unsubstituted isoindolinyl, substituted or unsubstituted substituted or unsubstituted quinoxalinyl, substituted or unsubstituted quinazolinyl, substituted or unsubstituted cinnolinyl, substituted or unsubstituted [2,3-c] or [3,2-c]-thienopyridyl, and the like. In some examples, R¹ is a substituted or unsubstituted fused C₄-C₉ heteroaryl, preferably unsubstituted indolyl.

In other specific examples, R¹ is selected from an alkyl sulfide, such as methyl sulfide.

In some aspects of Formula I′, I, II′, II, or II-A, R₂, R₃, and R₄ are independently absent or is hydrogen. In some embodiments, R₂ and R₄ are absent. In some embodiments, R₃ is hydrogen. In some embodiments, R₂ and R₄ are absent and R₃ is hydrogen.

In some aspects of Formula I′, I, II′, II, or II-A, X₁ is selected from C or N. In some embodiments, X₁ is C. In other embodiments, X₁ is or N.

In some aspects of Formula I′, I, II′, II, or II-A, X₂ is selected from C or N. In some embodiments, X₂ is C. In other embodiments, X₂ is or N.

In some aspects of Formula I′, I, II′, II, or II-A, X₃ is selected from C or N. In some embodiments, X₃ is C. In other embodiments, X₃ is or N.

In some aspects of Formula I′, I, II′, II, or II-A, X₁, X₂, and X₃ are the same or different. In some embodiments, X₁ and X₂ are both N. In some embodiments, X₁ and X₃ are both N. In some embodiments, X₂ and X₃ are both N. In some embodiments, X₁, X₂, and X₃ are all N. In some embodiments, X₁, X₂, and X₃ are all C.

In some aspects of Formula I′, I, II′, II, or II-A, X₄ is selected from C or N. In some embodiments, X₄ is C. In other embodiments, X₄ is or N.

In some aspects of Formula I′, I, II′, II, or II-A, X₅ is selected from C or S. In some embodiments, X₅ is C. In other embodiments, X₅ is or S.

In some aspects of Formula I′, I, II′, II, or II-A, X₆ is selected from C or O. In some embodiments, X₆ is C. In other embodiments, X₆ is or O.

In some aspects of Formula I′, I, II′, II, or II-A, X₄, X₅, and X₆ are the same or different. In some embodiments, X₄ and X₆ are both C. In some embodiments, X₄ and X₆ are both N. In some embodiments, X₄ is C and X₆ is O or N. In some embodiments, X₅ is S. In some embodiments, X₄, X₅, and X₆ are all C.

In some aspects of Formula I′, I, II′, II, or II-A, Y₁ is selected from CH or N. In some embodiments, Y₁ is CH. In other embodiments, Y₁ is or N.

In some aspects of Formula I′, I, II′, II, or II-A, Y₂ is selected from O or NH. In some embodiments, Y₂ is NH. In other embodiments, Y₂ is or O.

In some aspects of Formula I′, I, II′, II, or II-A, R₂, R₃, and R₄, when present, are independently selected from, hydrogen, halogen, hydroxyl, cyano, carboxyl, C₁-C₃ alkyl, C₁-C₃ haloalkyl, or cyclopropyl. When X₁ and X₂ are both N and R₂ and R₄ may be absent.

In some examples, X₃ can be C and R₃ is present and selected from hydrogen or C₁-C₃ alkyl.

In some aspects of Formula I′, I, II′, II, or II-A, R₅ₐ and R_{5b} are independently selected from, hydrogen, amino, substituted or unsubstituted aminoalkyl, substituted or unsubstituted C₁-C₆ alkyl, substituted or unsubstituted C₁-C₆ haloalkyl, substituted or unsubstituted cycloalkyl, substituted or unsubstituted heterocycloalkyl, or R₅ₐ and R_{5b} combine together with the atom to which they are attached to form a C₃-C₅ cycloalkyl or a C₂-C₄ heterocycloalkyl. For example, R₅ₐ and R_{5b} can be independently selected from, hydrogen, substituted or unsubstituted C₁-C₆ alkyl, or substituted or unsubstituted C₁-C₆ haloalkyl, substituted or unsubstituted cycloalkyl.

In some aspects of Formula I′, I, II′, II, or II-A, R₅ₐ and R_{5b} can combine to form a C₃-C₈ cycloalkyl or a C₂-C₇ heterocycloalkyl together with the atom to which they are attached.

In further aspects of Formula I′, I, II′, II, or II-A, R₅ₐ and R_{5b} are independently selected from, hydrogen, amino, aminoalkyl, C₁-C₆ alkyl, C₁-C₆ haloalkyl, cycloalkyl, heterocycloalkyl, or R₅ₐ and R_{5b} combine to form a carbonyl, a C₃-C₈ cycloalkyl or a C₂-C₄ heterocycloalkyl together with the atom to which they are attached, or one of R₅ₐ and R_{5b} combine with one of R₆ₐ and R_{6b} to form a C₃-C₈ cycloalkyl or a C₂-C₇ heterocycloalkyl together with the atom to which they are attached, or one of R₅ₐ and R_{5b} combine with R₁₂ to form a C₃-C₈ cycloalkyl or a C₂-C₇ heterocycloalkyl together with the atom to which they are attached. In some examples, R₅ₐ and R_{5b} combine to form a carbonyl, together with the atom to which they are attached.

In some aspects of Formula I′, I, II′, II, or II-A, R₆ₐ and R_{6b} are independently selected from, hydrogen, amino, substituted or unsubstituted aminoalkyl, substituted or unsubstituted C₁-C₆ alkyl, substituted or unsubstituted C₁-C₆ haloalkyl, substituted or unsubstituted cycloalkyl, substituted or unsubstituted heterocycloalkyl, or R₆ₐ and R_{6b} combine together with the atom to which they are attached to form a C₃-C₈ cycloalkyl or a C₂-C₄ heterocycloalkyl. For example, R₆ₐ and R_{6b} can be independently selected from, hydrogen, substituted or unsubstituted C₁-C₆ alkyl, or substituted or unsubstituted C₁-C₆ haloalkyl, substituted or unsubstituted cycloalkyl.

In further aspects of Formula I′, I, II′, II, or II-A, R₆ₐ and R_{6b} are independently selected from, hydrogen, amino, aminoalkyl, C₁-C₆ alkyl, C₁-C₆ haloalkyl, cycloalkyl, heterocycloalkyl, aryl, heteroaryl, or R₅ₐ and R_{6b} combine to form a C₃-C₈ cycloalkyl, a C₂-C₄ heterocycloalkyl, an aryl, or a heteroaryl, together with the atom to which they are attached.

In some aspects of Formula I′, I, II′, II, or II-A, R₅ₐ and R₆ₐ can combine together with the atom to which they are attached to form a C₂-C₇ heterocycloalkyl or C₂-C₇ heterocycloalkenyl.

In some aspects of Formula I′, I, II′, II, or II-A, both R₇ₐ and R_{7b} are O. In other aspects of Formula I′, I, II′, II, or II-A, R₇ₐ is O and R_{7b} is absent. In some aspects, R₇ₐ is O and R_{7b} is NH. In further aspects, R₇ₐ and R_{7b} are both absent.

In some aspects of Formula I′, I, II′, II, or II-A, R₈ can be selected from hydrogen or substituted or unsubstituted C₁-C₃ alkyl. For example, R₈ can be hydrogen.

In some aspects of Formula I′, I, II′, II, or II-A, R₉ and R₁₀ are independently selected from hydrogen or substituted or unsubstituted C₁-C₃ alkyl. For example, R₉ and R₁₀ can be hydrogen.

In some aspects of Formula I′, I, II′, II, or II-A, R₁₁ can be selected from substituted or unsubstituted C₁-C₃ alkyl. For example, R₁₁ can be methyl, ethyl, or propyl.

In some aspects of Formula I′, I, II′, II, or II-A, R₁₂ can be selected from sulfoximine, sulfonyl, sulfoxide, sulfur diimide, sulphonamide, amide, amine, hydroxyl, or one of R₅ₐ or R_{5b} combine with R₁₂ to form a C₂-C₇ heterocycloalkyl together with the atom to which they are attached. In some examples, R₁₂ can include an aliphatic or cyclic sulfoximine. In some examples, R₁₂ can include an aliphatic or cyclic ester. In some examples, R₁₂ can include an heteroatom such as, nitrogen. In some examples, R₁₂ can include an aliphatic or cyclic amine. In some examples, R₁₂ can include an aliphatic or cyclic sulfonyl. In some examples, R₁₂ can include an aliphatic or cyclic sulfonamide. In some examples, R₁₂ can include an hydroxyl group. R₁₂ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl.

In some aspects of Formula I′, I, II′, II, or II-A, the compound can have a structure represented by a formula:

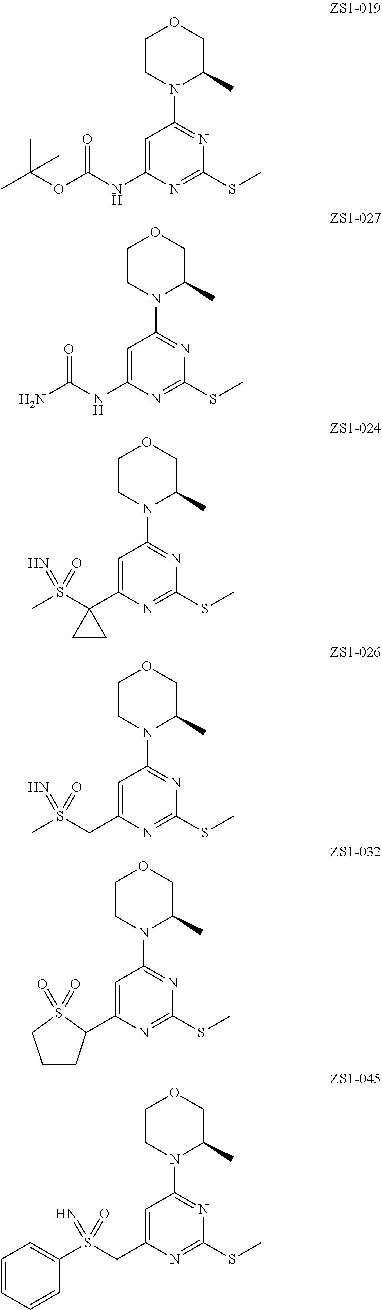

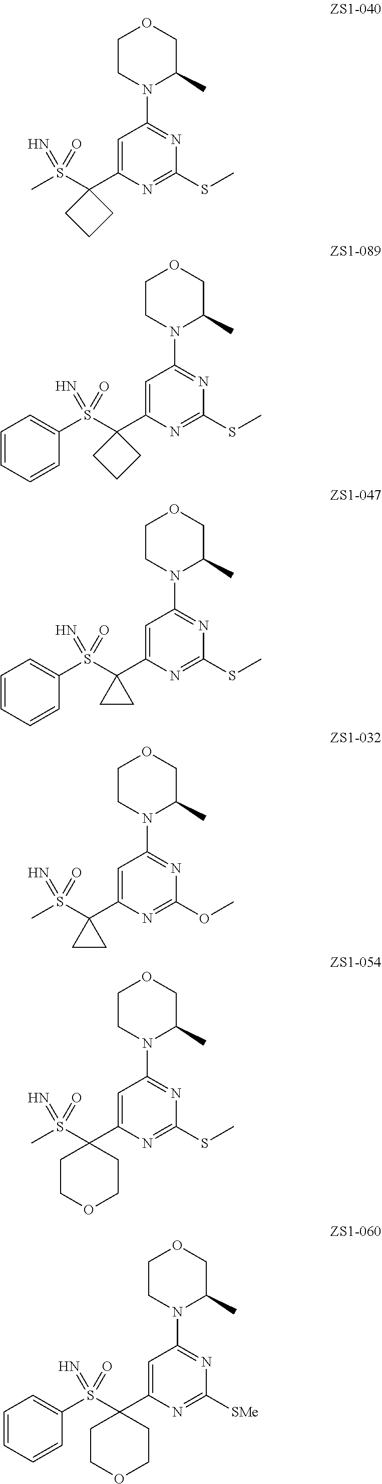

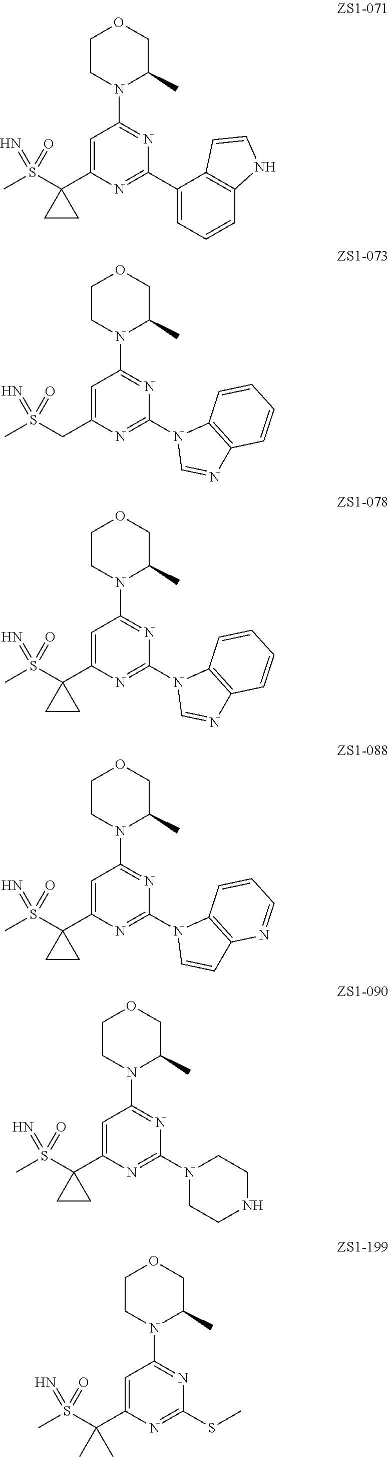

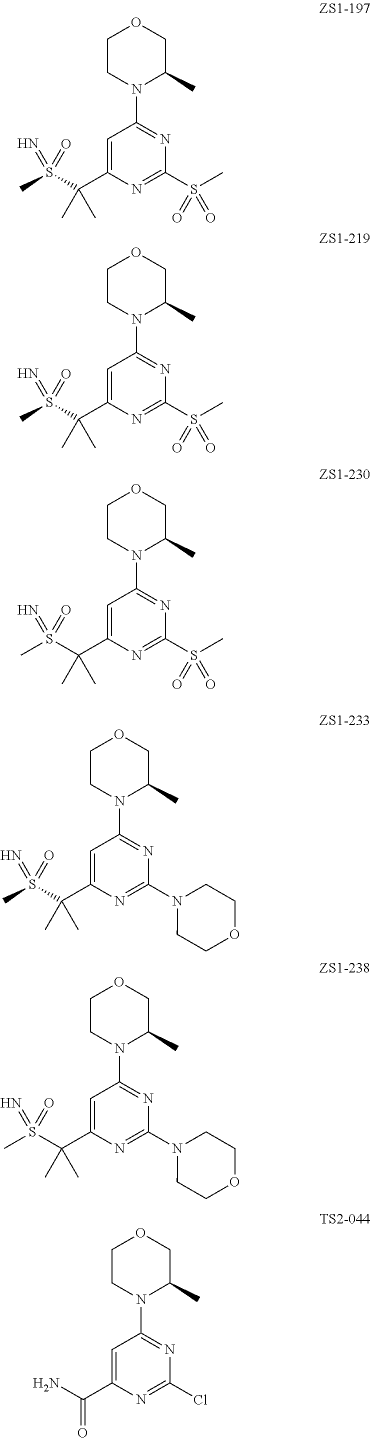

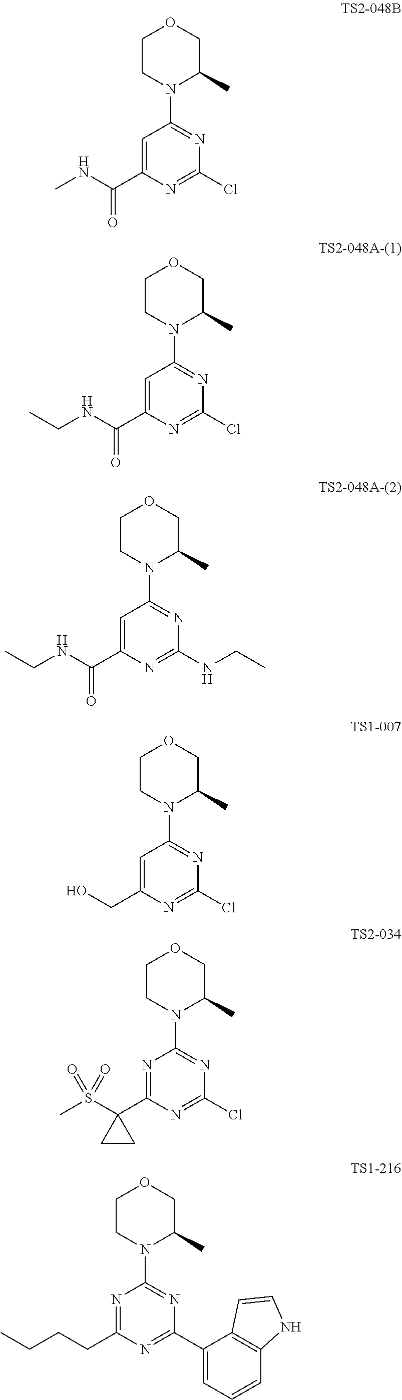

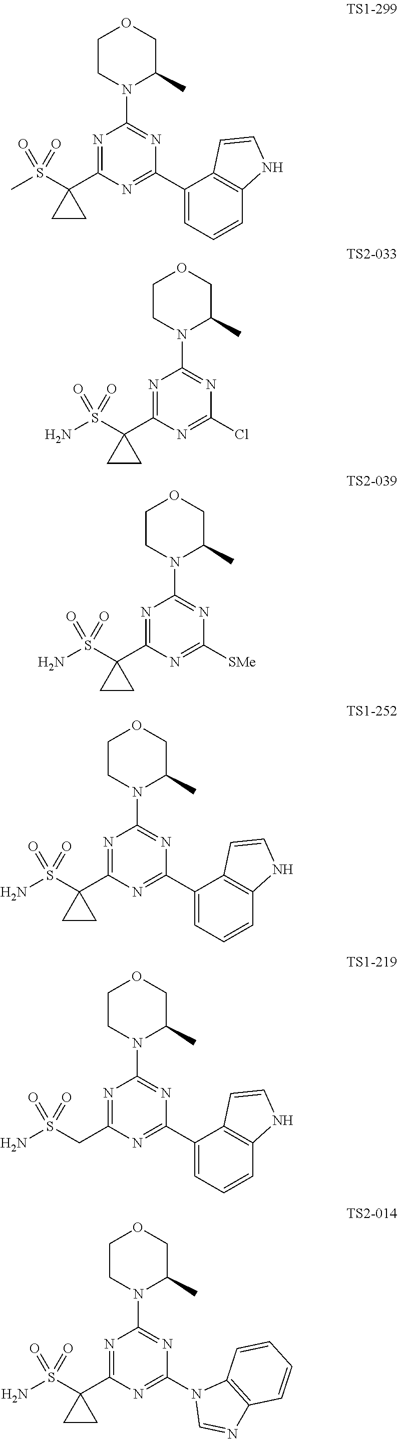

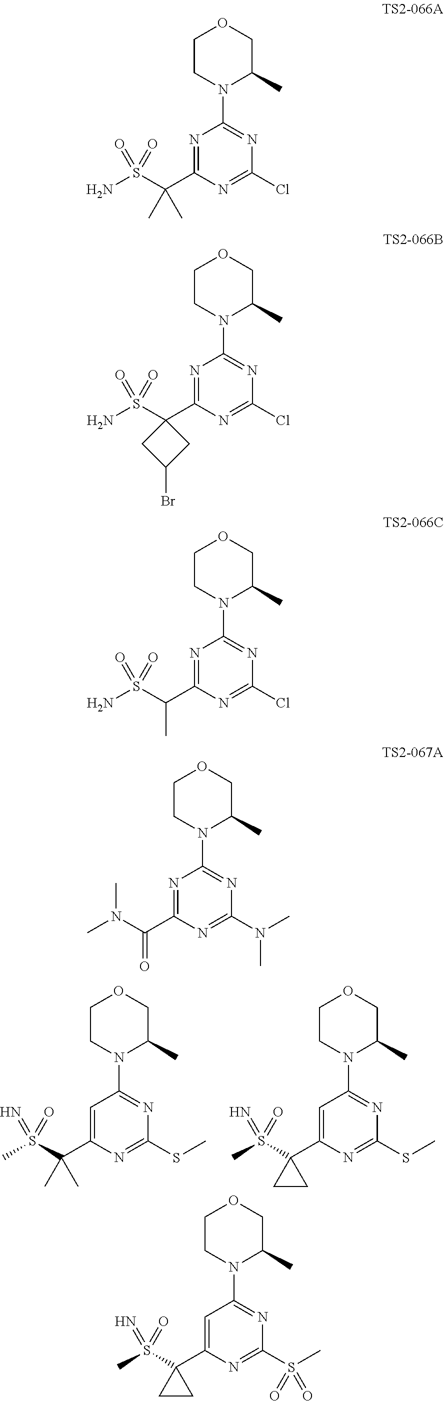

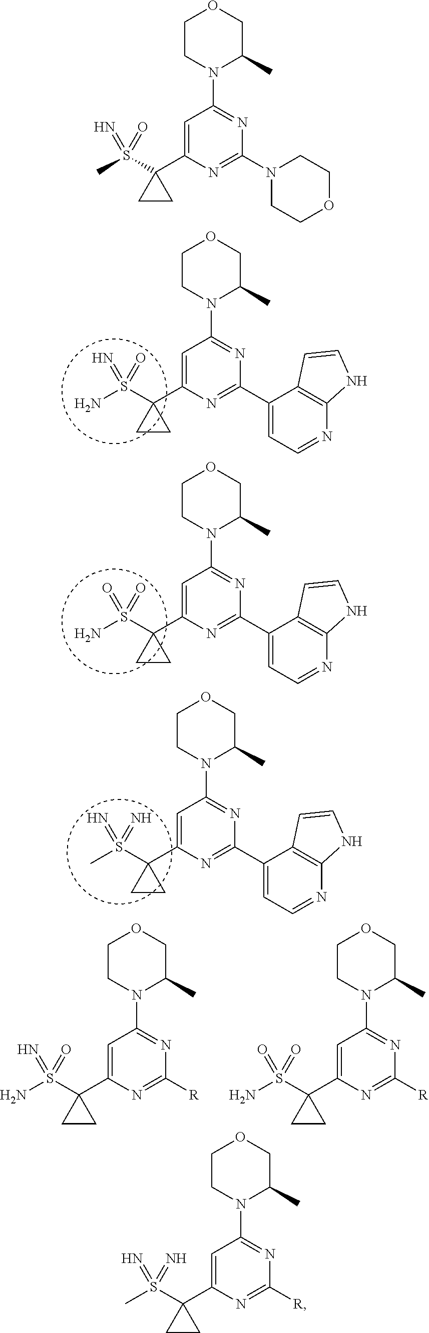

wherein R is selected from halogen, amine, alkylamine, thiol, sulfide, sulfonyl, sulfoximine, sulfoxide, sulfur diimide, sulphonamide, alkoxyl, cycloalkyl, heterocycloalkyl, aryl, or heteroaryl, wherein R is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl.

In some embodiments, the compound can be selected from:

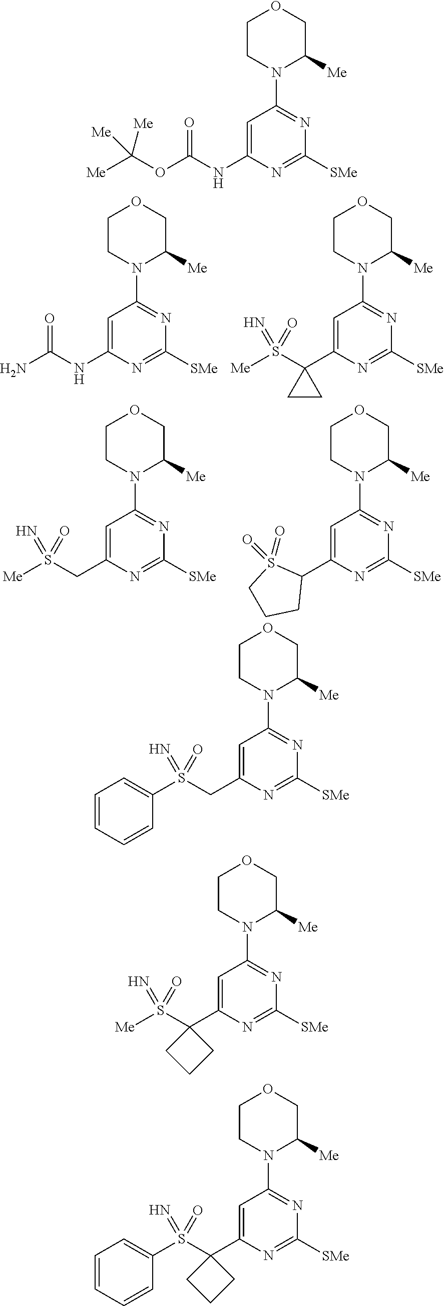

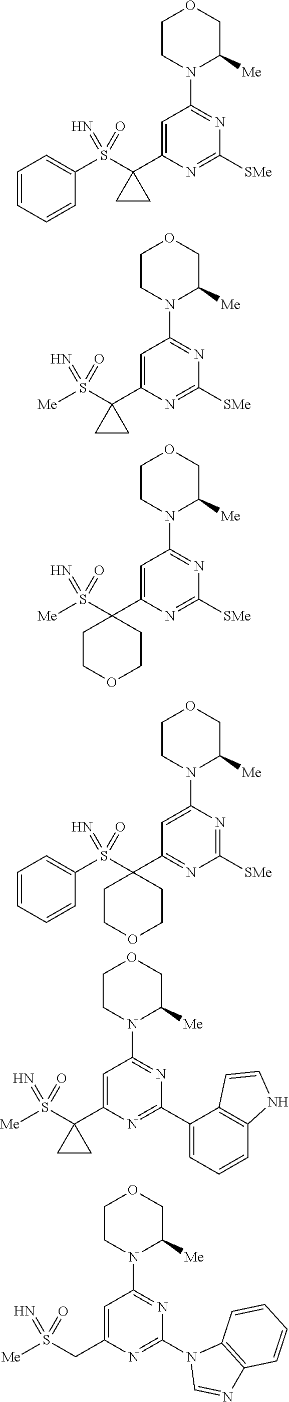

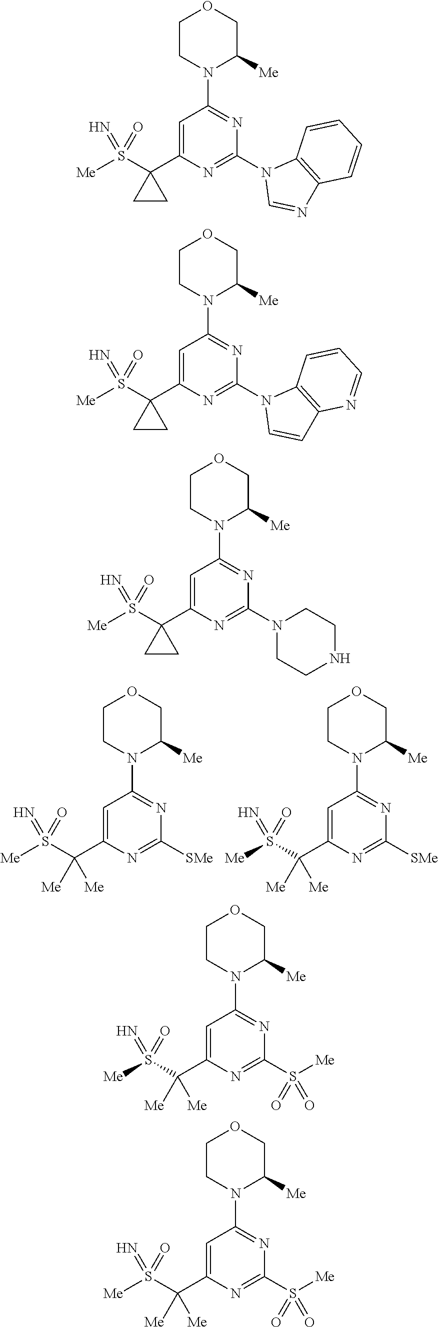

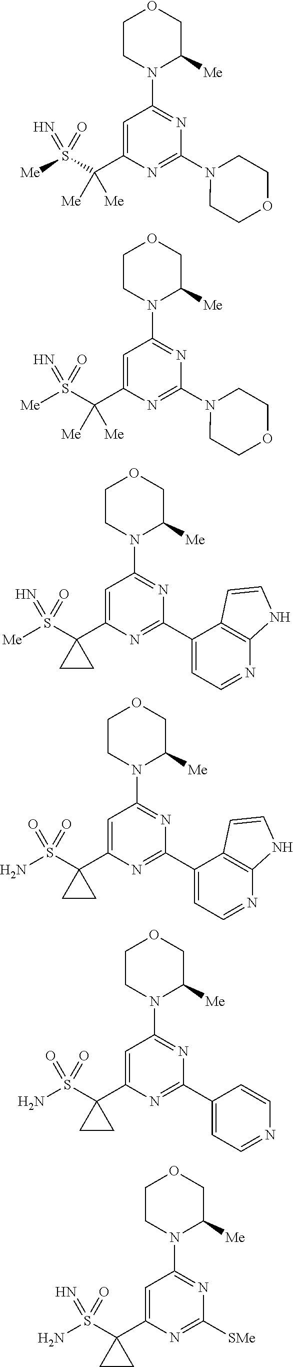

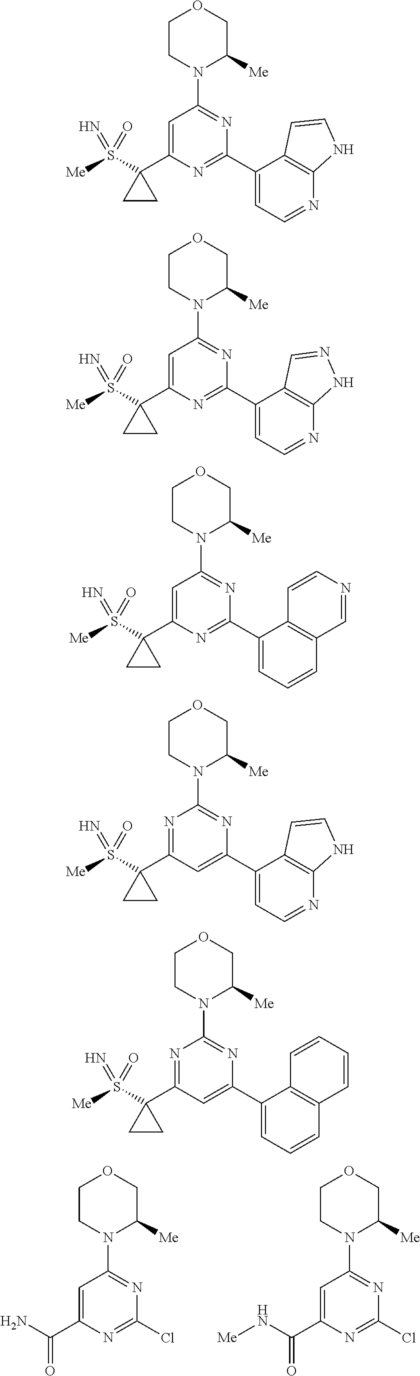

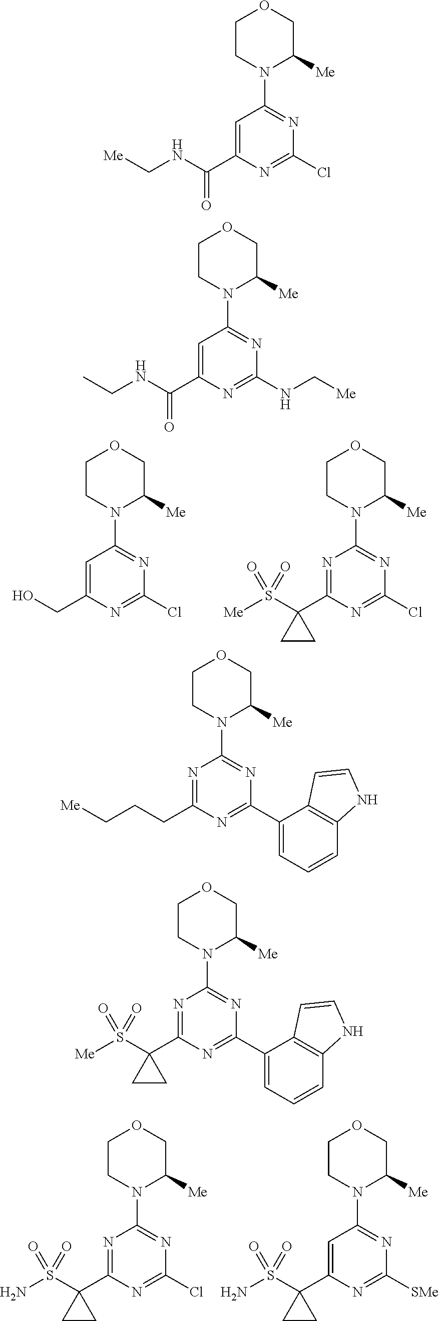

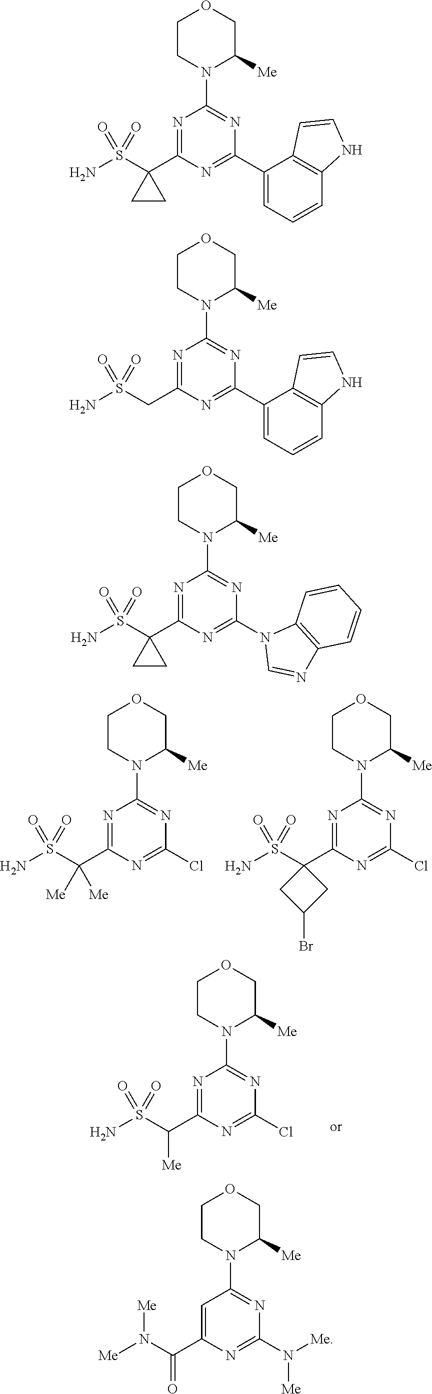

Pharmaceutical Compositions

The present disclosure provides pharmaceutical compositions comprising a compound of any one of Formulae I′, I, II′, or II, or a pharmaceutically acceptable salt, solvate, hydrate, polymorph, co-crystal, tautomer, stereoisomer, isotopically labeled derivative, or prodrug thereof), and optionally a pharmaceutically acceptable excipient. In certain embodiments, the pharmaceutical composition comprises a compound of Formula I, or a pharmaceutically acceptable salt thereof, and optionally a pharmaceutically acceptable excipient. In certain embodiments, the pharmaceutical composition comprises a compound of Formula II, or a pharmaceutically acceptable salt thereof, and a pharmaceutically acceptable excipient.

The compound is provided in an effective amount in the pharmaceutical composition. In certain embodiments, the effective amount is a therapeutically effective amount, a prophylactically effective amount, an amount effective for treating and/or preventing a disease (e.g., a disease described herein), an amount effective for treating a disease, an amount effective for treating and/or preventing a disease associated with a bromodomain-containing protein, an amount effective for treating a disease associated with a bromodomain-containing protein, an amount effective for treating and/or preventing a proliferative disease (e.g., a proliferative disease described herein), an amount effective for treating and/or preventing cancer (e.g., a cancer described herein), an amount effective for treating and/or preventing lung cancer (e.g., small-cell lung cancer or non-small-cell lung cancer), an amount effective for treating and/or preventing a benign neoplasm (e.g., a benign neoplasm described herein), an amount effective for treating and/or preventing a diseases or process associated with angiogenesis, or an amount effective for treating and/or preventing an inflammatory disease.

An effective amount of a compound may vary from about 0,001 mg/kg to about 1000 mg kg in one or more dose administrations for one or several days (depending on the mode of administration) In certain embodiments, the effective amount per dose varies from about 0,001 mg kg to about 1000 mg/kg, from about 0.01 mg/kg to about 750 mg/kg, from about 0.1 mg/kg to about 500 mg/kg, or from about 1.0 mg/kg to about 250 mg/kg.

Methods

The compounds and compositions disclosed herein are inhibitors of bromodomain-containing proteins. Transcription initiation factor TFIID subunit 1 (TAF1) and TAF1L are two bromodomain proteins. As a part of the STAGA complex containing TRRAP, GCN5, TFIID, CBP/P300, mediator and Sp1, TAF1 is susceptible to oncogenic activation by MYC. Moreover, TAF1 has been shown to block p53 activity, and inactivation of TAF triggers a DNA damage response. In addition, the TFIID complex, of which TAF1 is a significant member, is vital to stem cell reprogramming. In certain embodiments, the compounds disclosed herein bind to a bromodomain-containing protein, specifically to the bromodomain-containing protein TAF1 or TAF1L. In certain embodiments, the compounds show a greater binding affinity to the bromodomain-containing protein TAF1 or TAF1L than to one or more other proteins or one or more other bromodomain-containing proteins. The compounds disclosed herein are useful in the treatment of a disease associated with the activity a bromodomain-containing protein (e.g., a proliferative disease).

The selectivity of a compound for a bromodomain-containing protein over another protein (e.g., another bromodomain-containing protein) may be measured by the quotient of the Kd value of the compound in inhibiting the activity of the other protein over the IC₅₀ value of the compound in inhibiting the activity of the bromodomain-containing protein. The selectivity of a compound for a bromodomain-containing protein over another protein (e.g., another bromodomain-containing protein) may also be measured by the quotient of the Kd value of an adduct of the compound and the other protein over the ¾ value of an adduct of the inventive compound and the bromodomain-containing protein. In certain embodiments, the selectivity is at least about 1-fold, at least about 2-fold, at least about 5-fold, at least about 10-fold, at least about 30-fold, at least about 100-fold, at least about 300-fold, at least about 1,000-fold, at least about 3,000-fold, at least about 10,000-fold, at least about 30,000-fold, or at least about 1,000-fold. In certain embodiments, the selectivity is at most about 100,000-fold, at most about 10,000-fold, at most about 1,000-fold, at most about 100-fold, at most about 10-fold, or at most about 1-fold.

Bromodomain-containing protein is implicated in a wide range of diseases. Therefore, the inventive compounds are expected to be useful in treating and/or preventing diseases associated with bromodomain-containing proteins.

Provided herein are methods of treating or preventing a disorder of uncontrolled cellular proliferation associated with TAF1 dysfunction in a subject, comprising administering to the subject an effective amount of a compound or composition as disclosed herein. The compound can have a structure according to Formula I′, I, II′, II, or II-A, or selected form AZD6738, AZ20, or AZD3147. The subject can be a mammal such as a human. In some aspects, the subject has been identified to have a high mitotic activity, Mdm2-mediated degradation of the tumor suppressor p53, decreased levels of the protooncogene cMYC, or a combination thereof. In some examples, the disorder of uncontrolled cellular proliferation can be cancer.

Methods of inhibiting a bromodomain of TAF1 in at least one cell are also provided. The methods can include contacting the at least one cell with an effective amount of a compound or a pharmaceutical composition as described herein, such as a compound of Formula I′, I, II′, II, AZD6738, AZ20, or AZD3147.

Methods of treating or reducing the risk of a disorder of uncontrolled cellular proliferation associated with a bromodomain-containing protein in a subject are also disclosed. Methods of inhibiting the activity of a bromodomain-containing protein in a subject or cell are further also disclosed. Methods of inhibiting the binding of a bromodomain of a bromodomain-containing protein to an acetyl-lysine residue of a histone in a subject or cell are disclosed. The bromodomain-containing protein can be a bromo and extra terminal protein (BET). In some examples, the bromodomain-containing protein is TAF1 protein or TAF1L protein.

The methods disclosed herein can include administering to the subject a therapeutically effective amount of a compound or a pharmaceutical composition as described herein or a compound selected from AZD6738, AZ20, or AZD3147.

In the methods described herein, the methods can further comprise administering a second compound or composition, such as, for example, anticancer agents or anti-inflammatory agents to the subject. In some examples, the second compound is an anticancer compound such as those selected from a MDM2 inhibitor. Examples of MDM2 inhibitors include a Nutlin. Additionally, the method can further comprise administering an effective amount of ionizing radiation to the subject.

Methods of killing a tumor cell are also provided herein. The methods comprise contacting a tumor cell with an effective amount of a compound or composition as disclosed herein. The methods can further include administering a second compound or composition (e.g., an anticancer agent or an anti-inflammatory agent) or administering an effective amount of ionizing radiation to the subject.

Also provided herein are methods of radiotherapy of tumors, comprising contacting the tumor with an effective amount of a compound or composition as disclosed herein and irradiating the tumor with an effective amount of ionizing radiation.

Also disclosed are methods for treating oncological disorders in a patient. In one embodiment, an effective amount of one or more compounds or compositions disclosed herein is administered to a patient having an oncological disorder and who is in need of treatment thereof. The disclosed methods can optionally include identifying a patient who is or can be in need of treatment of an oncological disorder. The patient can be a human or other mammal, such as a primate (monkey, chimpanzee, ape, etc.), dog, cat, cow, pig, or horse, or other animals having an oncological disorder. Oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), multiple myeloma, neuroblastoma, benign neoplasm, and multiple myeloma.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

In some aspect, disclosed are methods for treating a tumor or tumor metastases in a subject by the administration to the subject a combination of at least one compound or composition as disclosed herein and at least one cancer immunotherapeutic agent. The disclosed compounds can be administered alone or in combination with a cancer immunotherapeutic agent. The subject can receive the therapeutic compositions prior to, during or after surgical intervention to remove all or part of a tumor. Administration may be accomplished via direct immersion; systemic or localized intravenous (i.v.), intraperitoneal (i.p.), subcutaneous (s.c.), intramuscular (i.m.), or direct injection into a tumor mass; and/or by oral administration of the appropriate formulations.

In specific examples, the type of cancer is breast cancer or lung cancer.

Administration

The disclosed compounds can be administered either sequentially or simultaneously in separate or combined pharmaceutical formulations. When one or more of the disclosed compounds is used in combination with a second therapeutic agent the dose of each compound can be either the same as or differ from that when the compound is used alone. Appropriate doses will be readily appreciated by those skilled in the art.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer for intracranial tumors; poly [bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi, et al., “Combined therapy with chemotherapeutic agents and herpes simplex virus type IICP34.5 mutant (HSV-1716) in human non-small cell lung cancer,” Human Gene Therapy, 1999, 10(18):17).

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.), and toxins of bacterial, fungal, plant, or animal origin (e.g., ricin, botulinum toxin, anthrax toxin, aflatoxin, jellyfish venoms (e.g., box jellyfish), etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

Kits

Kits for practicing the methods of the invention are further provided. By “kit” is intended any manufacture (e.g., a package or a container) comprising at least one reagent, e.g., anyone of the compounds described in Table 1. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

The kinase inhibitor AZD6738, a specific and potent inhibitor ATR, also selectively inhibits the second bromodomain of TAF1. While it has been reported that various kinase inhibitors inhibit BRD4 (dual BET-kinase inhibitors), AZD6738 is the first kinase inhibitor targeting bromodomains outside the BET family. High resolution co-crystal structures of TAF1 liganded with AZD6738 and close analogues was determined, revealing large conformational changes of the tandem bromodomain upon inhibitor binding. The knowledge of the precise binding pattern provides a new structural framework for the design of inhibitors with high potency and selectivity for TAF1 and the ability to concurrently inhibit ATR or other PI3K-related kinases. Studies in lung and colon cancer cell lines established that TAF1 inhibitors, including AZD6738, activate p53 and DNA damage response, and induce p21 and cell death. The ability to activate DNA damage signaling may reduce the need for DNA-damaging drugs that often cause significant toxicity.

The successful treatment of complex diseases such as cancer usually requires inhibition of multiple molecular targets or pathways. Most commonly this involves the combination of two or more drugs each engaging a different target or pathway. The classic rationale for multi-targeting by a combination therapy is to achieve greater overall efficacy by an additive or synergistic mechanism. This in turn may also allow the use of the two drugs at lower doses or for shorter periods, thereby potentially reducing the toxicity and cost for the patient. The use of multiple drugs is also thought to reduce the emergence of resistance to therapy, by inhibiting multiple signaling pathways, including any compensatory mechanisms that may bypass the targets of any of the component drugs. Resistance to kinase inhibitors has been discovered in many cancers and drug resistance remains the leading cause of cancer recurrence and metastasis. Moreover, recurrent cancers are more difficult to treat and bring unprecedented emotional and economic challenges.

The herein example and use of multi-targeted agents, particularly TAF1-ATR inhibitors, has several advantages over a conventional combination therapy including more predictable and manageable pharmacokinetic analysis, simplified dosing regimens, fewer complications from drug-drug interactions and higher patient compliance. Increasingly, the cost of targeted cancer therapies are such that combination of two such agents may become an issue. Several anti-cancer multi-targeting drugs have seen significant success in the clinic, most notably the kinase inhibitors sunitinib, dasatinib and lapatinib, vandetanib, all with tolerated toxicities. This disclosure has the potential to significantly impact current cancer treatments that involve administration of ATR or other PI3K-like kinase inhibitors. Dual TAF1-ATR inhibitors may be particularly useful against cancers that have acquired resistance to ATR inhibitors.

The rationale of this example is that TAF1 inhibition by small molecules is a viable strategy to alter the transcription machinery of cancer cells, particularly those evading p53-mediated DNA damage response and apoptosis. Thus, development and in-depth characterization of novel inhibitors that potently inhibit TAF1 and ATR are developed. This example integrates components from structural biology, cancer biology and medicinal chemistry for the development of dual TAF1-ATR inhibitors as cancer drugs.

Results

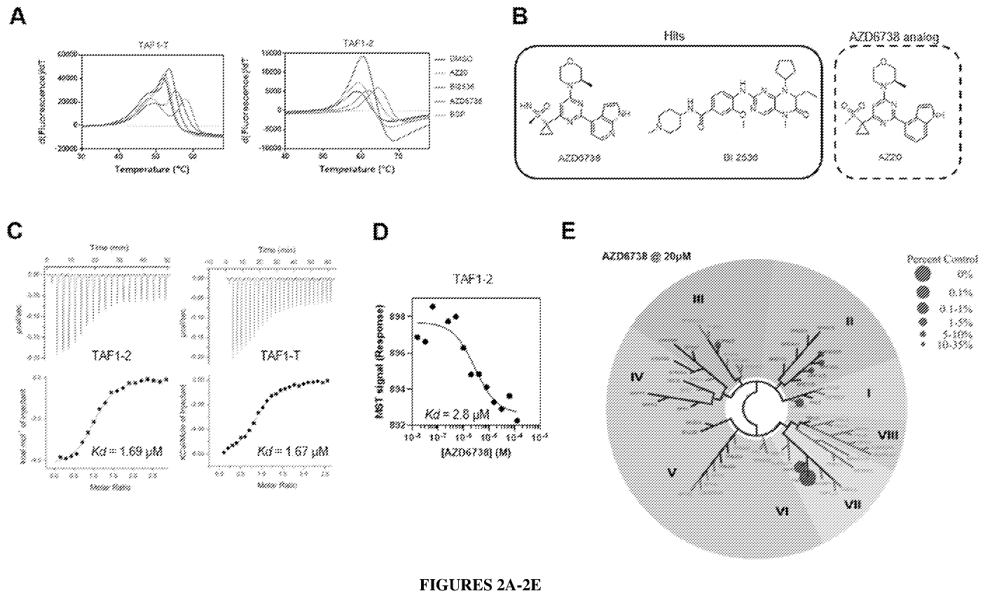

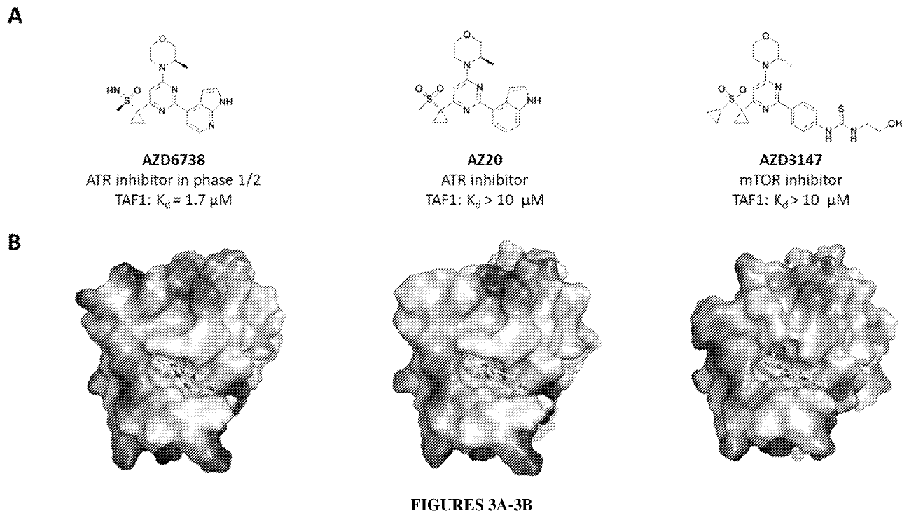

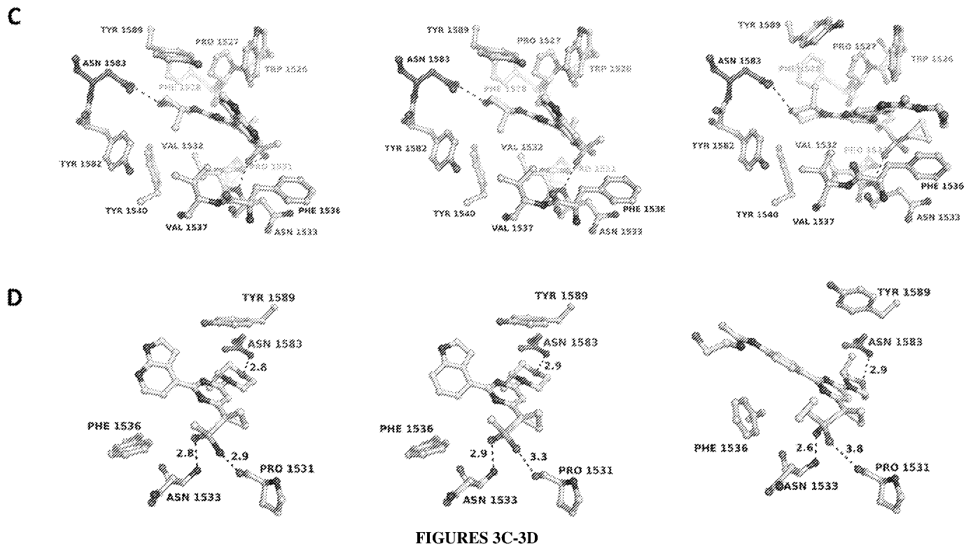

Discovery of a dual TAF1-ATR inhibitor. Following the discovery of dual BRD4-kinase inhibitors, targets of bromodomains outside the BET family, in particular the tandem bromodomain of the underexplored potential drug target TAF1 (TAF1-T) became a subject. Using differential scanning fluorimetry (DSF), crystallization-grade TAF1-T against a library of 418 kinase inhibitors were screened and AZD6738, a potent ATR kinase inhibitor was identified as a hit compound (Table 1). Follow-up studies including direct binding studies by isothermal microcalorimetry (ITC) confirmed AZD6738 as a TAF1 ligand with a Kd of 1.7 μM. Profiling of AZD6738 across a panel of 32 bromodomains by DiscoveRx revealed high selectivity for TAF1 and TAF1L (FIG. 2). Five other bromodomains (BRD2-2, BRD3-2, BRD4-2, CECR2 and WDR9-2) interacted weakly. Analogues of AZD6738, the ATR inhibitor AZ20 and the mTOR inhibitor AZD3147 (FIG. 3A) were obtained from commercial vendors, but they showed only weak binding potential for TAF1.

TABLE 1
Binding potential of AZD6738 and analogues towards TAF1. Bromosporine
(BSP) and BAY299 served as positive controls.

| | DSF (ΔTm, ° C.) | | ITC (Kd, nM) | | Reported kinase |
|---|---|---|---|---|---|
| Compounds | TAF1-T | TAF1-2 | TAF1-T | TAF1-2 | activity |
| BSP | 9 ± 0.4 | 4.2 ± 0.4 | 147 ± 17 | 52.6 ± 6.1 | unknown |
| BAY299 | 9.2 ± 0.1 | 2.7 ± 0.2 | 9.3 ± 2.9 | 46.7 ± 9.8 | unknown |
| AZD6738 | 5.8 ± 0.5 | 2.9 ± 0.2 | 1,670 ± 60 | 2,290 ± 200 | ATR |
| | | | | | (IC50 = 1 nM) |
| AZ20 | 0.01 ± 0.4 | 0.3 ± 0.3 | >10,000 | >10,000 | ATR |
| | | | | | (IC50 = 5 nM) |
| AZD3147 | 0.03 ± 0.3 | 0.2 ± 0.3 | >10,000 | >10,000 | mTOR |
| | | | | | (IC50 = 1.5 nM) |

AZD6738 and analogues bind to the second bromodomain of TAF1. Crystallographic studies with two TAF1 constructs were performed, TAF1-T encompassing BD1 and BD2, and TAF1-2 consisting of BD2 only. Compounds AZD6738, AZ20 and AZD3147 were subjected to co-crystallization screening campaigns, and conditions were established that allowed for the growth of X-ray grade crystals. High resolution structures determined between 1.7 and 2.3 Å resolution revealed that the three compounds bind to the KAc site of BD2 (FIG. 3B, C). All three compounds establish H-bonding interactions with the highly conserved Asn1583 through the morpholino oxygen. Comparison between AZD6738 and AZ20 showed a highly similar binding pattern except for the sulfoximine group of AZD6738, which establishes two H-bonds at the opposite end of the KAc site with the main chain amide nitrogen of Asn1533 and the oxygen of Pro1531 (FIG. 3D). The sulfonyl groups of AZ20 and AZD3147 establish only a single H-bond with Asn1533, while the other sulfonyl oxygen is incapable of interacting with Pro153, and in fact shifts away. These differences in H-bonding potential explain the considerably weaker binding activity of AZ20 and AZD6738. The opposite stereochemistry of the methylmorpholino moiety of AZD3147 and the additional cyclopropyl group cause a loss in shape complementarity with the KAc site. As a result, inhibitor binding is accompanied by substantial structural changes, particularly of residues Tyr1589 and Phe1536 (FIG. 3D).

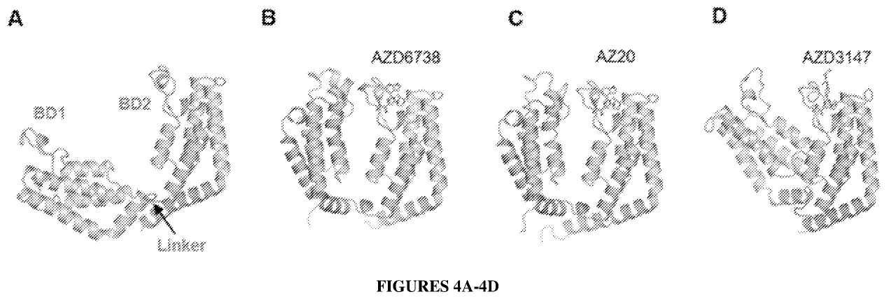

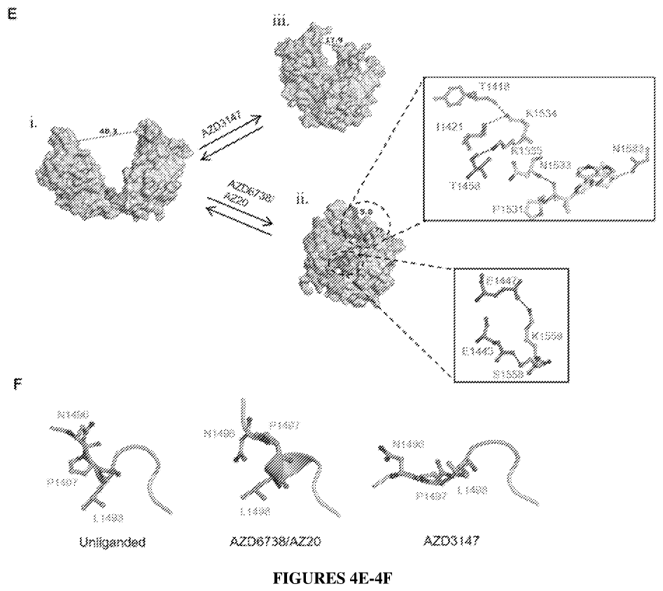

The tandem bromodomain of TAF1 shows large conformational changes upon inhibitor binding. Various structures of TAF-T using crystals grown in the absence and presence of inhibitors were determined. Comparison of unliganded and ligand structures revealed global conformational changes of TAF1-T resulting in distinct “open” and “closed” states (FIG. 4). The complex of TAF1-T with AZD6738 is the most compact structure, stabilized through inter-domain contacts of one flank of the KAc site (residues 1533-1535) with residues of BD1 (residues 1418, 1421 and 1458) (FIG. 4B). The TAF1-AZD3147 complex exists as a “semi-closed state”. AZ20 only yielded crystals with TAF1-BD2 but not with TAF1-T yet. The structural changes appear to be mediated through the linker region connecting BD1 with BD2, including a peptide flip around residues 1496-1498 (FIG. 4C). Combined, these findings support inhibitor-induced open-closed transitions in TAF1-T, albeit the molecular trigger for these changes is not clear yet and will be addressed in Aim 1. It is possible that the unliganded protein transitions between open and closed states, and that inhibitor binding merely stabilizes a certain conformation for crystal lattice formation.

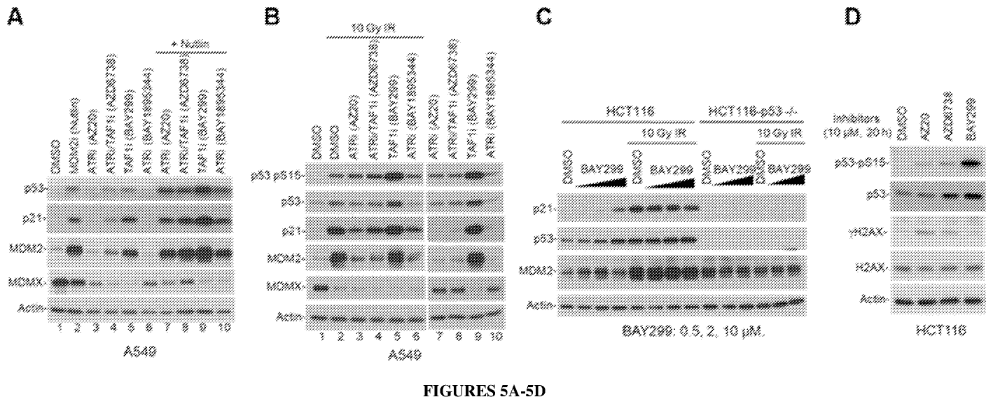

Inhibition of TAF1 bromodomain activates p53. To test the cellular effects of TAF1 bromodomain inhibition, A549 lung tumor cells were treated with inhibitors for 24 hours and analyzed for p53 pathway markers by western blot. The most potent TAF1 inhibitor BAY299 strongly induced p53 and p21 to levels similar to those caused by the MDM2 inhibitor Nutlin (FIG. 5A). AZD6738 also induced modest p53 and p21 increase, consistent with its moderate inhibition of TAF1. The ATR inhibitor AZ20 with weak TAF1 activity did not seem to induce p53 or p21. To determine whether p21 induction was mediated by p53, HCT116 colon cancer cells with and without p53 were tested for response to inhibitor. TAF1 inhibition did not induce p21 in the absence of p53, demonstrating that p53 activation mediates the induction of p21 (FIG. 5B).

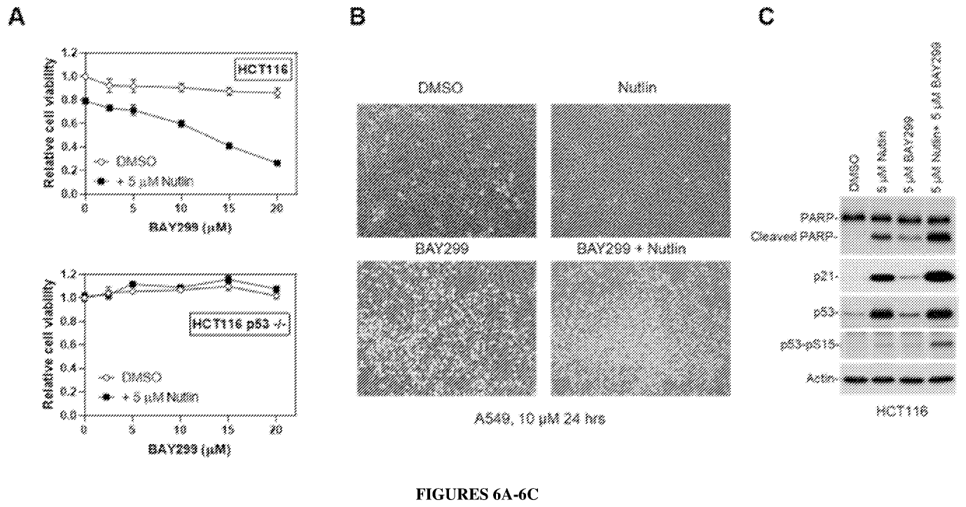

Inhibition of TAF1 bromodomain activates DNA damage signaling. P53 is activated by several mechanisms including DNA damage signaling, ribosomal stress, and oncogenic stress. TAF1 inhibition caused down regulation of MDMX, which is suggestive of DNA damage signaling. Analysis of DDR marker pSer15 level in p53 showed strong induction of Ser15 phosphorylation after TAF1 inhibitor treatment, similar to the effect of □-radiation (FIG. 6A). Furthermore, TAF1 inhibitors did not synergize with radiation in inducing p53 and p21, suggesting that the two treatments act through similar mechanism. Treatment with TAF1i inhibitors caused cell death in A549 cells, and further synergized with the MDM2 inhibitor Nutlin to induce cell death (FIG. 6B). The data suggest that TAF1 bromodomain is involved in DNA damage sensing or repair. Inhibition of TAF1 activates DNA damage signaling, which in turn activates p53. These results do not rule out TAF1 inhibition also has direct effect on its binding and phosphorylation of p53.

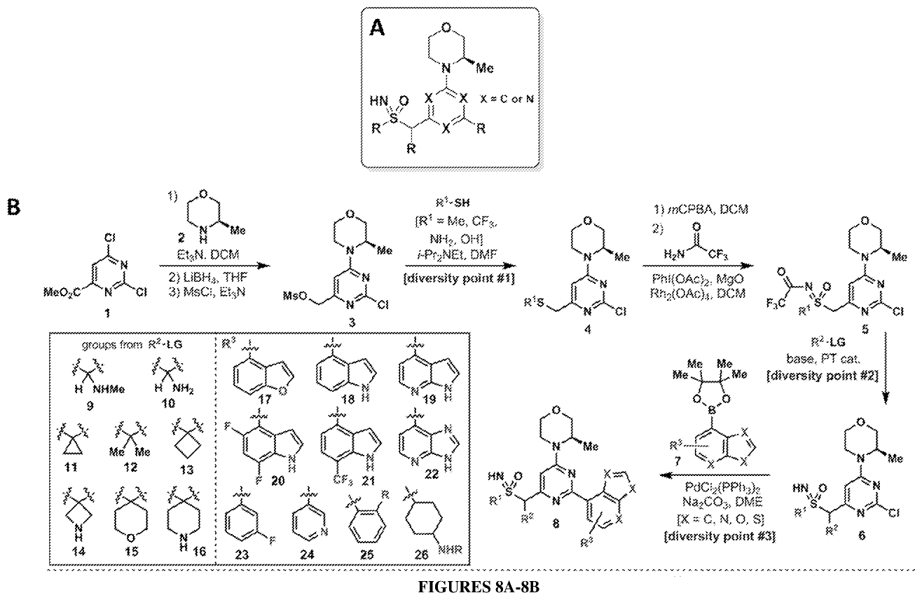

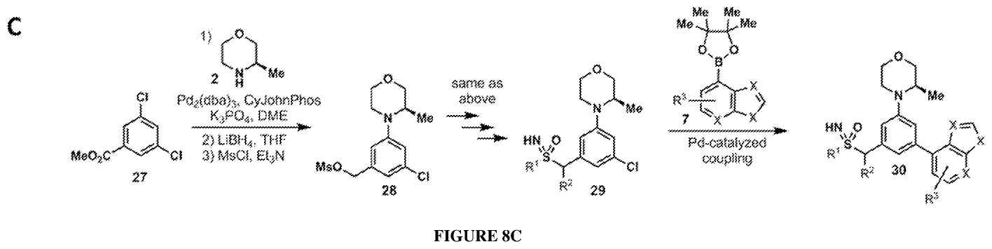

Structure-guided development of potent dual TAF1-ATR inhibitors. Using the new crystal structures of TAF1-inhibitor complexes, a series of analogues of AZD6738 for structure-activity relationship (SAR) studies has been proposed (FIG. 8). Synthesized compounds will be characterized by biochemical and biophysical studies to systematically increase binding affinity for TAF1 while maintaining potency for ATR and cellular activity. The structural basis of inhibitor-induced conformational changes will be analyzed by X-ray crystallography and fluorescence spectrometry using wild-type and engineered mutant constructs of TAF1.

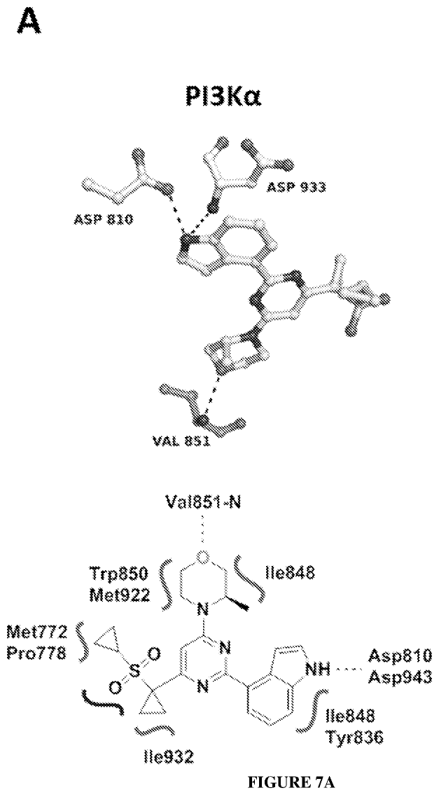

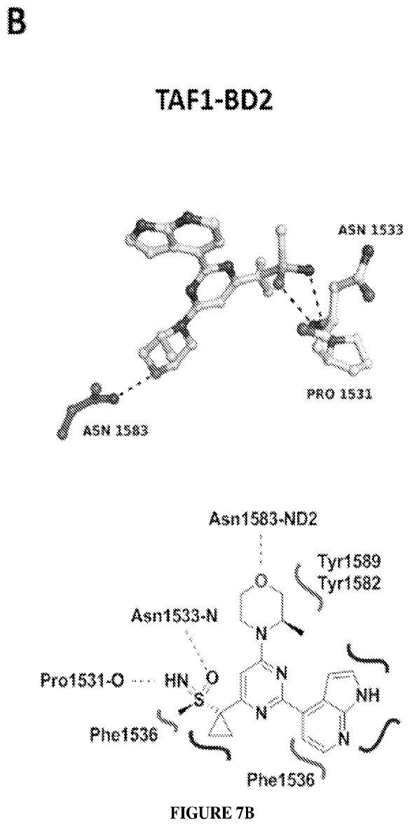

Design and synthesis of potent dual TAF1-kinase inhibitors. Comparison of the binding pattern of AZD6738 in TAF1-BD2 and that of a close analogue in PI3Kα shows that the methylmorpholino group is especially favorable for binding to the conserved asparagine in the KAc site and to the hinge region of the ATP site (FIG. 7). The pyrrolopyrimidine group is solvent exposed in TAF1 while the similar indole group interacts with ATP site residues. The sulfoximine group is also favorable for binding to the KAc site (FIG. 3D) while it is partly exposed to solvent in PI3Kα. Thorough analysis of the H-bonding and VDW interactions revealed modification sites in AZD6738 that could increase the binding potential towards TAF1 substantially while maintaining high kinase activity (FIG. 8). For structure-activity relationship (SAR) studies, analogues were designed based on the principle chemical scaffold of FIG. 8A employing different diversity-oriented synthesis routes (FIG. 8B, C).

The established literature route for AZD6738 can be used as the basis for a diversity-oriented synthesis of a library of analogs with initial modifications on the sulfoximine (R¹), pyrimidine (R²) and fused heterocycle (R³). The groups at these three positions can vary by lipophilicity, steric, electronic, and hydrogen-bonding properties. The route can commence with the coupling of commercially-available pyrimidine 1 with enantiopure morpholine 2; reduction and mesylation of the resulting alcohol will give intermediate 3 (FIG. 8B). Treatment of intermediate 3 with various sulfides (R¹—SH, or other nucleophilic sulfur species such as SCF₃) can serve as the first diversification step and afford pyrimidine 4. Conversion to protected sulfoximine 5 will proceed by oxidation with mCPBA to give the corresponding sulfoxide followed by rhodium-catalyzed imidation with trifluoroacetamide. (A) Treatment of sulfoximine 5 with base and an electrophilic species (R²-LG, LG=leaving group) can install functionality at the second point of diversification to yield pyrimidine 6. Amines can be installed via electrophilic amination (9-10), while the alkyl groups can be added through classic nucleophilic displacement chemistry (11-16). A variety of heterocycles (core compound 7, derivatives 17-22) and arenes (23-25) can be installed in the third diversification step through a Suzuki coupling (or other standard cross-coupling reactions as needed) to afford the final analog 8. In addition, saturated rings (e.g. 26) can be installed through a nickel-catalyzed reductive cross-coupling with the corresponding secondary alkyl bromides. An alternate route is described in FIG. 8C, where the pyrimidine core of AZD6738 is replaced by a phenyl ring. The overall route and diversification steps are identical, except for the first step, which can proceed via a Buchwald-Hartwig cross-coupling of commercially-available arene 27 with enantiopure morpholine 2. The final Suzuki reactions using aryl chlorides and heterocyclic boronic esters to give analog 30 have precedent; however, if the reaction conditions prove to be unduly harsh, the dibromo analog of arene 27 can be used instead. The proposed route is expected to generate mixtures of diastereomers in several cases (e.g. 5 and 6). The stereoisomers can be separated by chromatography (chiral HPLC, if required) to ensure that only single purified compounds are used for crystallographic and biological testing.

In vitro characterization of dual TAF1-ATR inhibitors. Compounds can be systematically analyzed for improvement of binding activity and inhibitory potency towards TAF1 using biochemical and biophysical methods. An E. coli expression system suitable for the production of crystallization-grade TAF1-T, TAF1-BD2 and TAF1-BD1 from 4 liter cultures has been established. Purification can be performed by FPLC using a combination of nickel-affinity, ion exchange and size-exclusion chromatography.

Structural biology. Compounds can be co-crystallized with TAF1 constructs using established or new crystallization conditions. Mutant proteins can be generated and crystallized to probe the role of certain residues in the global conformational changes observed upon inhibitor binding (FIG. 4B, C). Fluorescence Resonance Energy Transfer (FRET) studies can be employed to detect and monitor inhibitor-induced structural changes of the tandem bromodomain. Inhibitors capable of locking TAF1-T in a closed conformation may exert superior properties over mere competitive inhibitors of the KAc site, particularly if the induced conformational changes are incompatible for the interaction with cellular, lysine-acetylated binding partners. To assess the structure-activity relationship of ATR inhibition, mutant PI3Kα constructs can be used as models for the ATR kinase domain using the baculovirus/insect cell expression methodology.

Differential scanning fluorimetry (DSF). DSF is based on the detection of differences in protein stability at increasing temperatures in the presence of a ligand and will be used to assess the binding potential of compounds. The stabilizing effect of ligand binding to the KAc site of BRDs is logarithmically proportional to the binding activity, with thermal shifts larger than 6° C. typically corresponding to submicrolar binding affinity. The Protein Thermal Shift™ Dye Kit in a StepOnePlus™ Real-Time PCR System from Applied Biosystems can be used.

Isothermal titration calorimetry (ITC). Thermodynamic profiles and dissociation constants between compounds and TAF1 can be determined using a MicroCal-iTC200 instrument housed in the Moffitt Chemical Biology Core. This method is well established in the characterization of several BRD inhibitors including our own studies with TAF1 (FIG. 2B). Thermodynamic signatures can be used to prioritize compounds for further development.

qPCR-based BRD binding assay. Another method for the determination of dissociation constants of BRD-ligand complexes is offered by DiscoveRx Corp. The assay is based on competition between test compound and immobilized high-affinity ligand for binding to the KAc site of BRDs. The amount of TAF1 remaining on an immobilized high-affinity ligand in the presence of ligand is measured using a quantitative real-time polymerase chain reaction (qPCR) method that detects the associated DNA label tagged to the BRD.

AlphaScreen assay. The interaction of TAF1 with new inhibitors can also be monitored using the colloidal, bead-based Amplified Luminescence Proximity Homogeneous Assay (AlphaScreen assay), which is based on the displacement of histone peptides from BRDs. Reaction Biology Corp. which offers the AlphaScreen assay for dose-response analysis on all BET BRDs.

Kinase activity assays. Biochemical evaluation of inhibitory potency against kinases can be determined using a highly sensitive ³³P-radiolabeled (“hotspot™”) assay by Reaction Biology. Activity of compounds against ATR can also be assessed in cell-based signaling studies using suitable biomarkers.

Inhibitor selectivity assays To assess target selectivity, compounds with highest biochemical and cellular activities can be profiled against panels of BRDs and kinases using the services of DiscoveRx and/or Reaction biology, as we have done previously.

ADME and PK properties. Compounds selected in each series can be subjected to experimental ADME to demonstrate that the compounds have properties suitable for use as chemical probes and eventually as clinical agents. This will include in vitro ADME analysis human liver microsomal stability (and CYP 3A4 and 2D6 inhibition, IC₅₀ ideally >10 uM and hERG binding IC₅₀ (HERG)/Cmax ideally >30 (Sanford-Burnham, Exploratory Pharmacology Core); aqueous solubility, ideally 1-2 mg/ml), plasma stability and permeability (Moffitt Chemistry Core). Later stage compounds will be assessed for in vivo ADME properties (oral bio-availability >30%, half-life >6 h and AUC) in collaboration with the Moffitt Translational Research Core to ensure that the best compounds are used in the proposed animal experiments.

Specific Aim 2. Functional analysis of ATR/TAF1 dual inhibitors Preliminary results identified p53 activation and p21 induction as biomarkers for TAF1 bromodomain inhibitors. Furthermore, inhibition of TAF1 bromodomain cooperates with MDM2 inhibition to induce cell death. These findings suggest that the bromodomain of TAF1 is a potential target for activating p53 and inducing cell death. P53 activation can also serve as a convenient marker for monitoring the cell entry and efficacy of ATR/TAF1 dual inhibitors developed.

Determine the ability of dual inhibitors to activate p53. The levels of p53, pSer15 at p53, p21, MDM2, MDMX can be determined by western blot upon treating the A549 cells with compounds at 0.1-10 μM for 24 hrs. pSer15 at p53 will serve as a marker for DNA damage signaling. The TAF1 inhibitor BAY299 can serve as a positive control. The p53-dependence of p21 induction will be further analyzed by comparing the response of HCT116 cells with and without p53. The results can provide evidence for the cellular penetrance of the new compounds and a ranking of their ability to activate p53.

TAF1-p53 binding is mediated by the bromodomains of TAF1 and two acetylated lysine residues on p53. To determine whether the dual inhibitors block TAF1-p53 binding, TAF1-p53 complex in treated cells can be detected by co-immunoprecipitation of TAF1 with p53. Cells can also be treated with the pan-HDAC inhibitor TSA to stimulate p53 acetylation, which should result in increased TAF1-p53 interaction. The analysis can confirm the ability of TAF1 inhibitors to inhibit TAF1-p53 interaction in vivo. To test whether the dual inhibitors prevent TAF1-mediated phosphorylation of p53 Thr55, A549 cells can be treated with □-radiation in the presence and absence of TAF1 inhibitors. P53 can be analyzed using a generic p53 and a pThr55-specific antibody. Inhibition of TAF1-p53 binding should prevent DNA damage-induced Thr55 phosphorylation.

Lastly, to verify the ability of dual inhibitors to inhibit ATR in vivo, A549 cells treated with hydroxyurea (to activate ATR) and dual inhibitors at a range of concentrations can be analyzed for the phosphorylation of Chk1 S345 (marker for ATR activity) by Western blot. AZD6738 can be used as benchmark to confirm the inhibition of ATR in vivo.

Investigate the p53-dependence of dual inhibitor bioactivity. To test the activity of dual ATR/TAF1 inhibitor, A549 cells can be treated at a range of concentrations for 72 hrs. Cell proliferation and viability can be analyzed using CellTiter Blue assay. The mechanism of cell death can be analyzed by Western blot for cleaved PARP and by FACS analysis of sub-2N cell population. Furthermore, long-term viability can be analyzed using clonogenic assay in which drug treated cells are plated at low density for 2 weeks to observe proliferation and colony formation. The dual inhibitors can be compared with AZ20, BAY299, and AZD6738 to determine whether the modifications improved potency.

To determine whether p53 activation contributes to the dual inhibitor anti-tumor activity, p53 in A549 cells can be knocked out using CRISPR/Cas9 approach. The ability of the dual inhibitors to induce cell death in A549 and A549-p53−/− cells can be compared to determine the significance of p53 activation. Furthermore, the results can be confirmed using HCT116 and HCT116-p53−/− cells. MDM2 inhibitors are in clinical trials in combination with DNA damaging agents and mitogenic kinase inhibitors. Results suggest that TAF1 synergizes with MDM2 inhibition to induce cell death. Therefore, the ability of the dual inhibitors to cooperate with Nutlin can be analyzed using MTT and FACS assays described above. These experiments can identify a top-ranked dual inhibitor for testing in animal model.

Determine the role of TAF1 bromodomains in regulating transcription. Being a subunit of a general transcription complex, TAF1 inhibition is thought to affect the expression of large number of genes. However, given its ability to activate p53, it may be important to distinguish indirect effects from p53. RNA-seq analysis of TAF1-specific and ATR/TAF1 dual inhibitors using p53-null cells to identify genes regulated by TAF1. A549 and A549-p53−/− cells treated with vehicle can be performed. TAF1 inhibitor and ATR/TAF1 dual inhibitor for 24 hrs can be subjected to RNA-seq analysis to compare global changes in gene expression. The top-ranking genes differentially expressed after TAF1 inhibition can be analyzed using DAVID and PANTHER websites to determine pathways that are most affected. Selected genes can be verified using RT-PCR. The results can reveal how much of the TAF1i effect is mediated by p53 activation and whether activation of specific pathways (such as those mediating apoptosis) explains the cell death observed after TAF1 inhibition.

Investigate the ability of dual inhibitor to inhibit tumor growth in vivo. AZD6738 has good bioavailability. It is believed the dual inhibitor developed based on this structure will retain similar performance. The identification of p53 as a biomarker for TAF1 inhibition provides a convenient marker to analyze the dual inhibitor activity in vivo. A549 subcutaneous tumor xenografts labeled with a p53-responsive luciferase reporter (BP100-luc) can be generated. Established tumors can be treated with the optimized dual inhibitor by i.p. injection at 10-50 mg/kg to the mouse xenograft. Live bioluminescence imaging can be performed 4-24 hrs after injection to detect compound access to tumor cells and activation of p53. The kinetics and duration of p53 activation can be determined. Controls can use AZD6738 that has weak TAF1i activity. P53 and p21 induction in the treated tumors will also be analyzed by Western blot to confirm the bioluminescence analysis.

After validating the ability of the dual inhibitor to activate p53 in tumors, a cohort (˜12) of established A549 tumors can be treated with the compound at an optimal p53 activation dose every other day for 20 days. A control cohort can be treated with vehicle only. A second control cohort will be treated with AZD6738. Tumor growth can be measured with a caliper.

The results can reveal whether dual inhibition of ATR and TAF1 is more potent than ATR inhibition in suppressing tumor growth or inducing regression. Further experiments can be performed in A549-p53−/− cells to determine whether p53 expression is necessary for the dual inhibitor to exhibit superior activity over AZD6738. The results can determine whether the dual inhibitor should be selectively used in patients without p53 mutations.

Summary: Taken together, the findings reported herein suggest that inhibiting the bromodomains of TAF1 is a promising approach to target the transcription machinery of cancer cells through an epigenetic mechanism of action.

### Example 2: Small Molecule Inhibitors of TAF1 Stabilize Distinct Conformational States of the Tandem Bromodomain and Elicit a p53-Dependent DNA Damage Response

ABSTRACT: Bromodomain-containing proteins regulate chromatin remodeling and gene transcription through interaction with acetylated lysine residues. Transcription initiation factor TFIID subunit 1 (TAF1) initiates preinitiation complex formation and cellular transcription. Therefore, TAF1 is a potential target to develop small molecule inhibitors for diseases arising from dysregulated transcription such as cancer. Reported herein that the ATR kinase inhibitor AZD6738 is a bona fide inhibitor of the second bromodomain of TAF1. Xray crystallography and small-angle X-ray scattering studies established that different inhibitors stabilize distinct structural states of the TAF1 tandem bromodomain through “open-closed” transitions and dimerization. In cancer cell line models, TAF1 inhibitors elicited a DNA damage response that resulted in p53 activation and increased cytotoxicity in combination with MDM2 inhibitor Nutlin. Combined, the data provide new insights into the mechanism of action of TAF1 inhibitors and their potential to invoke DNA damage signaling and p53-mediated cell death in cancer.

INTRODUCTION: Reported herein are that ATR kinase inhibitor AZD6738 and derivatives thereof are bona fide inhibitors of the second bromodomain (BD2) of TAF1. Using X-ray crystallography and small-angle Xray scattering (SAXS) it was demonstrated that different TAF1 inhibitors stabilize distinct structural states of the tandem bromodomain (TAF1-T). It was shown that small molecule inhibitors of TAF1 elicit a DNA damage signaling response leading to p53 activation in cancer cell line models.

Results

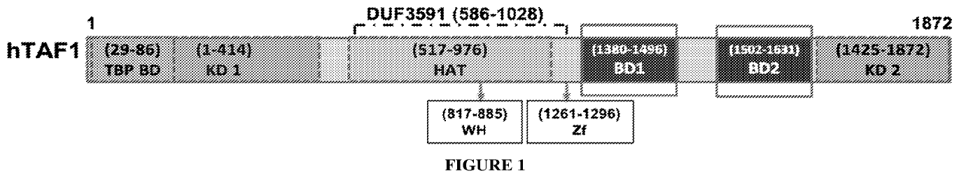

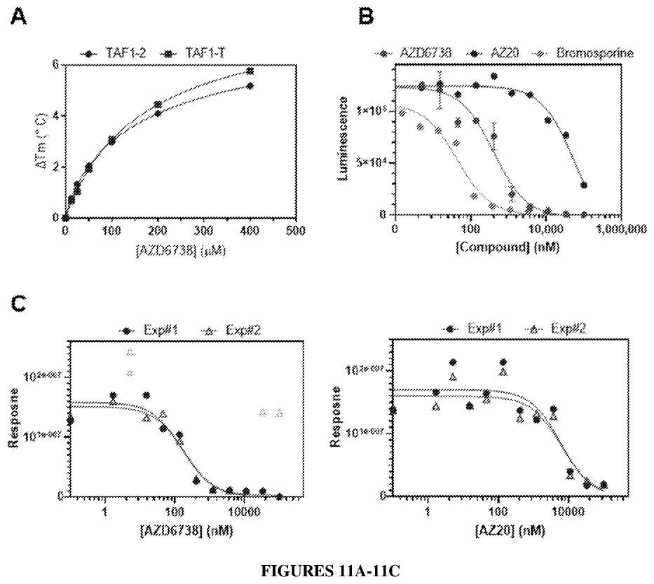

Discovery of dual TAF1-kinase inhibitors: TAF1 is an essential subunit of TFIID which is not considered an oncogene. Moreover, recent studies suggest that chemical inhibition of the BRDs of TAF1 is insufficient to prevent cancer cell growth. Therefore, the inventors sought to identify small molecule inhibitors that can inhibit TAF1 and a kinase with the potential to produce synergistic or synthetic lethal effects. Studies were performed with two TAF1 constructs, TAF1-2 consisting of BD2 only and TAF1-T encompassing BD1 and BD2 (FIG. 1). TAF1-T was screened against a library of 418 kinase inhibitors using differential scanning fluorimetry (DSF) to detect compounds with binding potential. Two hit compounds, AZD6738, a potent ATR kinase inhibitor in advanced clinical trials and BI2536, a potent PLK1 inhibitor, were identified (FIG. 2A-B, Table 2). AZD6738 induced thermal shifts of TAF1-T and TAF1-2 with ΔTm values of 3.1 and 3.2° C., while BI2536 showed weaker binding potential with ΔTm values of 1.2 and 1.7° C., respectively. AZ20, a close analog of AZD6738 and a dual ATR/mTOR inhibitor, was present in the compound library but was not flagged as a hit compound due to small thermal shifts of <0.5° C. Isothermal titration calorimetry (ITC) and microscale thermophoresis (MST) confirmed that AZD6738 binds to TAF1-2 with Kd values of 1.69 and 2.8 μM, respectively (FIG. 2C-D, Table 2, Table 3). The binding affinity of AZD6736 for TAF1-T as determined by ITC was almost identical to that of TAF1-2 (Kd=1.67 μM). Unbiased assays by commercial services, AlphaScreen (Reaction Biology) and pPCR-based BromoScan (DiscoverX), showed significantly higher binding activity with IC₅₀ and Kd values of 427 nM and 175 nM, respectively (Table 2, FIG. 11).

The discrepancy in binding potential is presumably due to differences in assay methodology and protein preparation. Throughout this example crystallization-grade TAF1-2 and TAF1-T constructs were used; therefore, the inventors have confidence in the values obtained by direct binding studies showing inhibitory potential against TAF1. Profiling of AZD6738 across a panel of 32 bromodomains revealed high selectivity for the second bromodomain of TAF1 and TAF1L (FIG. 2E, Table 4). Five other bromodomains (BRD2-2, BRD3-2, BRD4-2, CECR2 and WDR9-2) weakly interacted with AZD6738.

TABLE 2
Binding potential of TAF1 inhibitors

| | DSF ΔTₘ¹ | | ITC K [symbol] ² | | | Bromoscan ™ K [symbol] ¹ | AlphaScreen EC [symbol] ¹ |
|---|---|---|---|---|---|---|---|
| | (° C.) | | (nM) | | | (nM) | (nM) |
| Compound | TAF1-2 | TAF1-T | TAF1-1 | TAF1-2 | TAF1-T | TAF1-2 | TAF1-2 |
| AZD6738 | 3.1 ± 0.1 | 3.2 ± 0.1 | NB | 1,690 ± 190 | 1,670 ± 80 | 175 ± 19.2 | 426.5 ± 187 |
| AZ20 | 0.3 ± 0.2 | 0.1 ± 0.1 | ND | >25,000 | >25,000 | 6,167 ± 151 | >25,000 |
| BI2536 | 1.7 ± 0.1 | 1.2 ± 0.1 | ND | ND | ND | ND | ND |
| Bromosporine | 5.7 ± 0.1 | 10.8 ± 0.3 | ND | 52.6 ± 6.1 | 147 ± 17 | ND | 44.1 ± 16.6 |
| BAY299 | 3.6 ± 0.2 | 11.2 ± 0.1 | 4,600 ± 1,100 | 46.7 ± 9.8 | Kd₁ = | ND | ND |
| | | | | | 9.3 ± 2.9 | | |
| | | | | | Kd₂ = | | |
| | | | | | 2,100 ± 700 | | |

¹Standard deviation from two experiments.
²Standard error of the mean from data fitting. Bromoscan ™ by DiscoveRx and AlphaScreen by Reaction Biology.
NB: No binding. ND: Not determined. [symbol]
indicates data missing or illegible when filed

TABLE 3
ITC experimental conditions and thermodynamic parameters of TAF1 Inhibitors

| | [P] | | [L] | | Temperature | Kd | | ΔH | TΔS | ΔG |
|---|---|---|---|---|---|---|---|---|---|---|
| Protein | (μM) | Ligand | μm | Assay buffer | (K) | (nM) | N | (kcal/mol) | | |
| TAF1-T | 250 | AZD6738 | 20 | 50 mM HEPES | 298.15 | 1.670 ± 60 | 1.11 | −5.82 | 2.26 | −7.88 |
| | 250 | AZ20 | 20 | (pH 7.5), 0.05% | | >25,000 | NA | −4.13 | 3.97 | −8.09 |
| | 250 | BSP | 20 | Tween20 | | 146.6 ± 13.9 | 1.09 | −14.55 | −5.22 | −9.33 |
| | | | | | | 9.26 ± 2.9 | [symbol] 3 | −10.54 | 0.41 | −10.95 |
| | 250 | BAY299 | 20 | | | 2,100 ± 700 | [symbol] 1.2 | −0.60 | 7.15 | −7.75 |
| TAF1-2 | 350 | AZD6738 | 25 | 50 mM HEPES | | 1,690 ± 190 | 0.99 | −4.36 | 3.26 | −7.62 |
| | 250 | BSP | 25 | (pH 7.5), 0.05% | 288.15 | 52.8 ± 6.1 | 0.90 | −10.02 | −0.42 | −9.60 |
| | 250 | BAY299 | 25 | Tween20 | | 46.7 ± 9.8 | 0.96 | −12.31 | −2.23 | −10.08 |
| TAF1-1 | 250 | [symbol] AY299 | 20 | 50 mM HEPES | 298.15 | 4.600 ± 1.100 | 1.29 | −3.52 | 3.76 | −7.28 |
| | | | | (pH 7.5), 300 mM | | | | | | |
| | | | | NaCl, 0.05% | | | | | | |
| | | | | Tween20 | | | | | | |

[symbol]

indicates data missing or illegible when filed

TABLE 4
Selectivity profiling of AZD6738 against
32 bromodomains (by DiscoveRx)

| | % Inhibition |
|---|---|
| Bromodomains | AZD at 20,000 nM |
| ATAD2A | 0 |
| ATAD2B | 0 |
| BAZ2A | 0 |
| BAZ2B | 0 |
| BRPF2/BRD1 | 6 |
| BRD2(1) | 46 |
| BRD2(2) | 84 |
| BRD3(1) | 60 |
| BRD3(2) | 82 |
| BRD4(1) | 50 |
| BRD4(2) | 69 |
| BRD7 | 14 |
| BRD9 | 13 |
| BRDT(1) | 56 |
| BRDT(2) | 38 |
| BRPF1 | 11 |
| BRPF3 | 20 |
| CECR2 | 92.3 |
| CREBBP | 20 |
| EP300 | 36 |
| FALZ | 11 |
| GCN5L(2) | 27 |
| PBRM1(2) | 5 |
| PBRM1(5) | 0 |
| PCAF | 18 |
| SMARCA2 | 15 |
| SMARCA4 | 7 |
| TAF1(2) | 99.8 |
| TAF1L(2) | 98 |
| TRIM24(PHD, Bromo.) | 6 |
| TRIM33(PHD, Bromo.) | 0 |
| WDR9(2) | 84 |

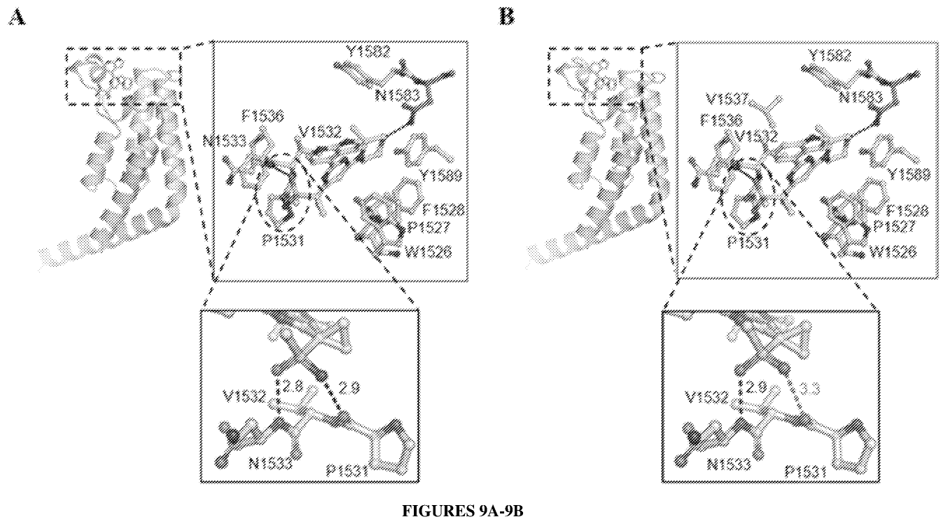

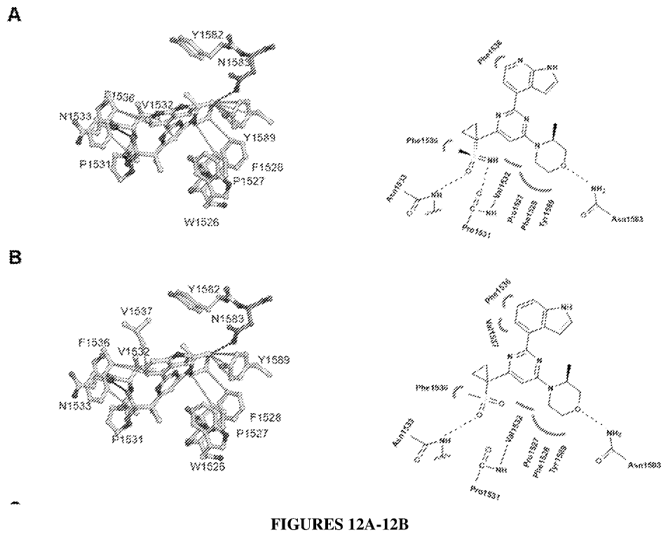

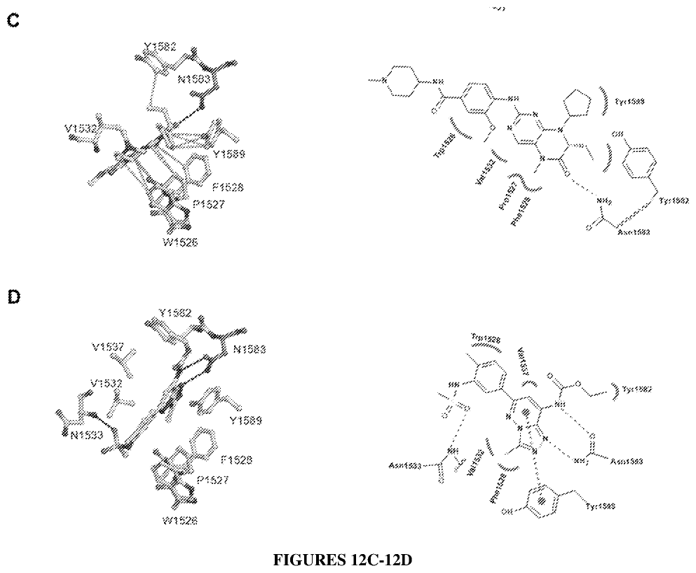

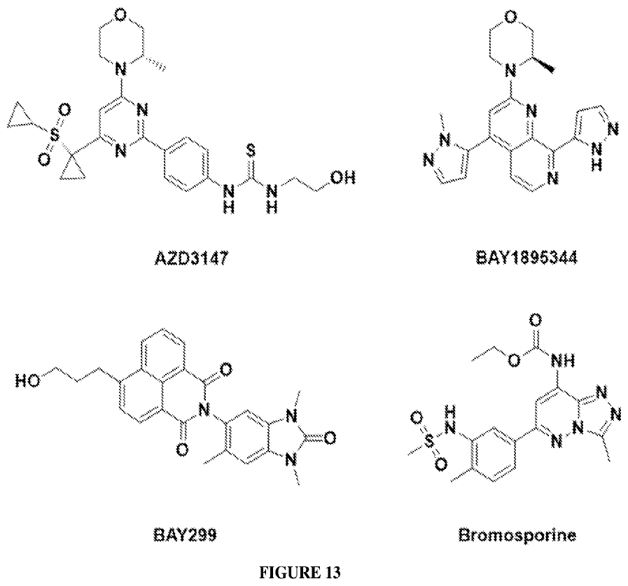

Structural basis of TAF1 inhibition by kinase inhibitors: To elucidate the binding mode of phosphatidylinositol 3-kinase-related kinase (PI3KK) inhibitors in TAF1, crystallographic studies were performed with TAF1-2 and TAF1-T. Compounds were subjected to co-crystallization screening campaigns, and conditions were established for the growth of X-ray grade crystals in different space groups depending on the ligand present. Co-crystal structures of TAF1-2 determined between 1.6 and 2.5 Å resolution revealed that AZD6738 and AZ20 bind to the KAc site through canonical H-bonding interaction of the morpholine oxygen with the side chain of conserved residue Asn1583 (FIG. 9A-B, FIG. 12). AZD6738 and AZ20 showed a highly similar binding pattern except for the sulfoximine group of AZD6738, which establishes H-bonds with the main chain amide nitrogen of Asn1533 and the carbonyl oxygen of Pro1531. The sulfonyl group of AZ20 is less suited for this favorable H-bonding pattern and in fact shifts away from Pro1531, reflected in considerably weaker binding affinity for TAF1. Comparison of the binding modes of 3-methylmorpholine-containing inhibitors in PI3KK kinase and TAF1 BRD reveals that the methylmorpholine moiety improves binding to the hinge region of the ATP site and to the conserved asparagine in the KAc site (FIG. 7A-B). The pyrrolopyridine moiety is located deep in the ATP site but is partly exposed to solvent in the KAc site, while the sulfoximine group is partly exposed to solvent in the ATP site but establishes H-bonds in the KAc site. The differences in binding pattern reveal sites that could be modified towards inhibitors with differential activities for TAF1 BRD over ATR kinase.

BI2536 binds to TAF1-2 through canonical H-bonding interaction with conserved residue Asn1583 similar to that observed in BRD4. While BI2536 shows high shape complementarity with the KAc site of BRD4, it is less well positioned in TAF1 (FIG. 12). In TAF1, BI2536 is in hydrophobic VDW contact with only one flank of the KAc site and lacks H-bonding potential with other residues, which explains the substantially weaker binding potential for TAF1 (Kd>10 uM) compared to BRD4 (Kd<100 nM). A co-crystal structure was also determined for TAF1-2 liganded with bromosporine, a pan-BRD inhibitor with a Kd value of 53 nM for TAF1, which was used as a positive control in library screening and hit validation studies (FIG. 12). The binding pose of bromosporine in the KAc site of TAF1-2 is similar to that of previously reported TAF1L, a testis-specific variant of TAF1.

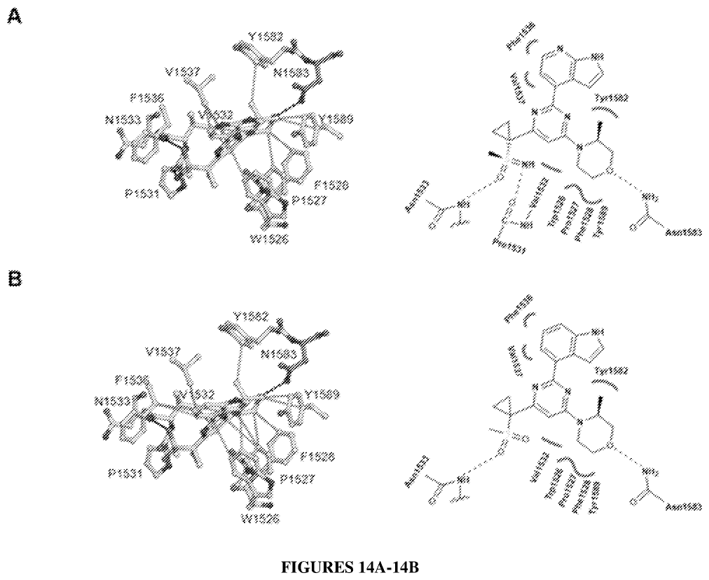

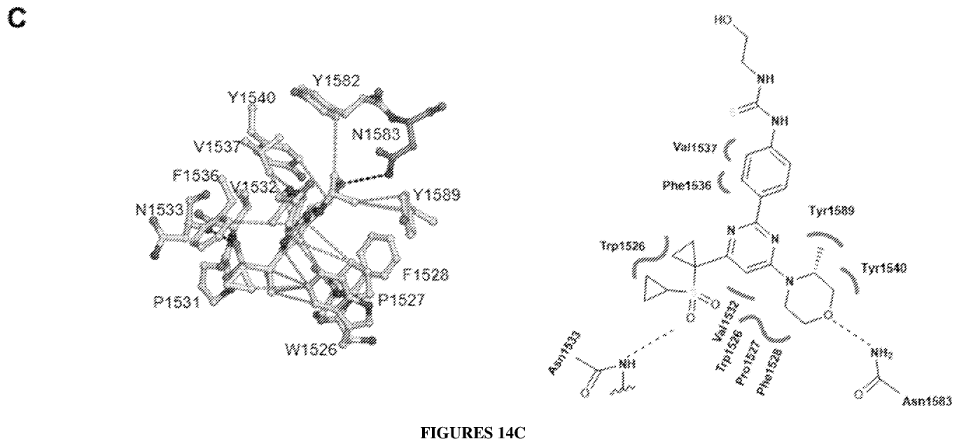

Dual TAF1-PI3KK inhibitors induce global structural changes of TAF1-T: Next, co-crystal structures of TAF1-T with 3-methylmorpholine containing PI3KK inhibitors AZD6738, AZ20 and the mTOR inhibitor AZD3147 were determined (FIG. 12). All three inhibitors occupied solely the KAc site of BD2 (FIG. 4A-D), and the binding mode of AZD6738 and AZ20 in TAF1-TBD2 was identical to that of isolated TAF1-2 (FIG. 14). For AZD3147, the opposite stereochemistry of the 3-methylmorpholine moiety and the additional cyclopropyl group connected to the sulfonyl moiety resulted in loss of shape complementarity with the KAc site. Inhibitor binding was accompanied by substantial structural changes, particularly of residues Tyr1589 and Phe1536 (FIG. 14).

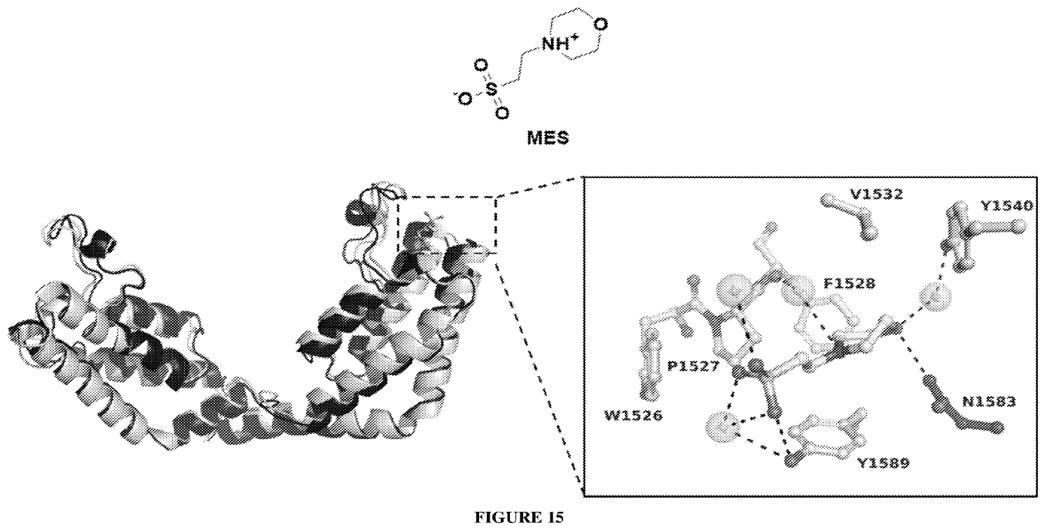

Previously reported crystal structures showed different conformational states of TAF1-T in unliganded states, bivalent inhibitor bound state and in complex with histone chaperone CIA/ASF1. Whether these structural changes are influenced by monovalent small molecule inhibitors was not known. In the crystal structure, unliganded TAF1-T adopts an “open” conformation (FIG. 4A) in which BD1 and BD2 are separated by ˜40 Å from each other. The complexes of TAF1-T with AZD6738 and AZ20 crystallized in compact “closed” conformational states (FIG. 4B-C), in which BD1 and BD2 interact with each other through residues close to the KAc site (K1534 with T1428 and 11421, K1535 with T1458) and approximately midway of the BD1-BD2 interface (K1559 with E1447 and S1558 with E1443) (FIG. 4E). TAF1-T liganded with AZD3147 showed a “semi-closed” conformation (FIG. 4D), in which the KAc sites are ˜18 Å distant from each other (FIG. 4E). These structural changes are likely mediated by the linker region connecting BD1 and BD2, including a peptide flip around residues 1496-1498 (FIG. 4F). Notably, the open state of unliganded TAF1-T was also observed in a different space group, in which 2-(N-morpholino)ethanesulfonic acid (MES) was bound in the KAc site of BD2 through direct interaction of the morpholine oxygen with N1583 (FIG. 15). This finding indicates that stabilization of more compact structural states requires a more elaborate binding pattern of inhibitors in the KAc site than simple canonical interaction of fragment-like compounds like MES. Each structure determination of TAF1-T required different conditions for crystal growth resulting in different space groups. Therefore, it appears that the conformational state of protein-inhibitor complex in solution dictates the crystal lattice, and not vice versa. Combined, the crystallographic data indicate inhibitor-induced structural transitions of TAF1-T.

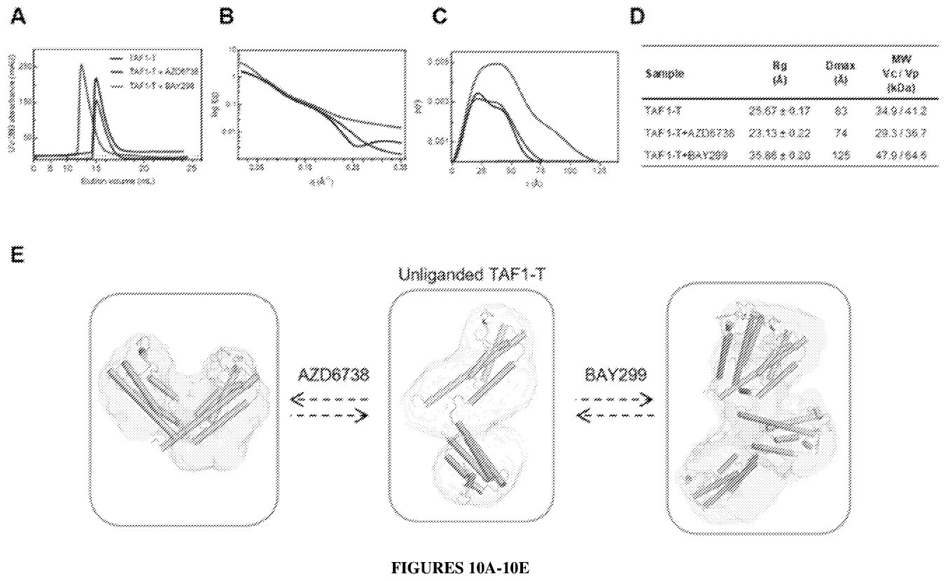

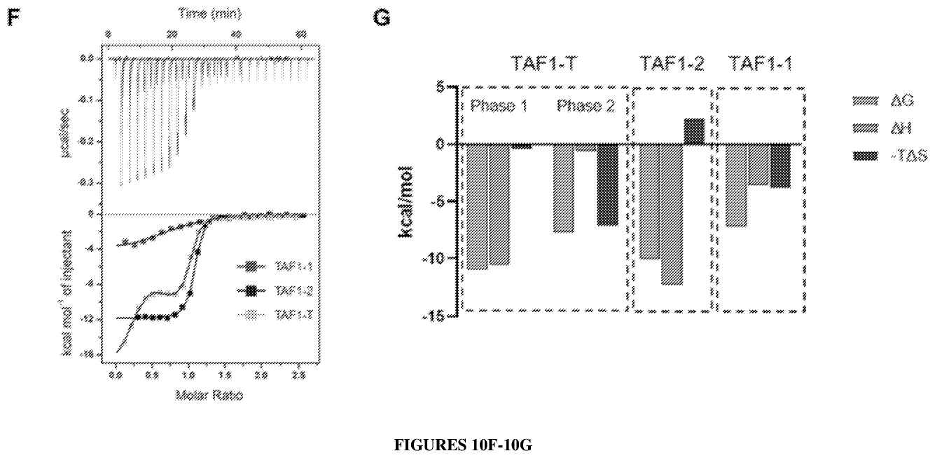

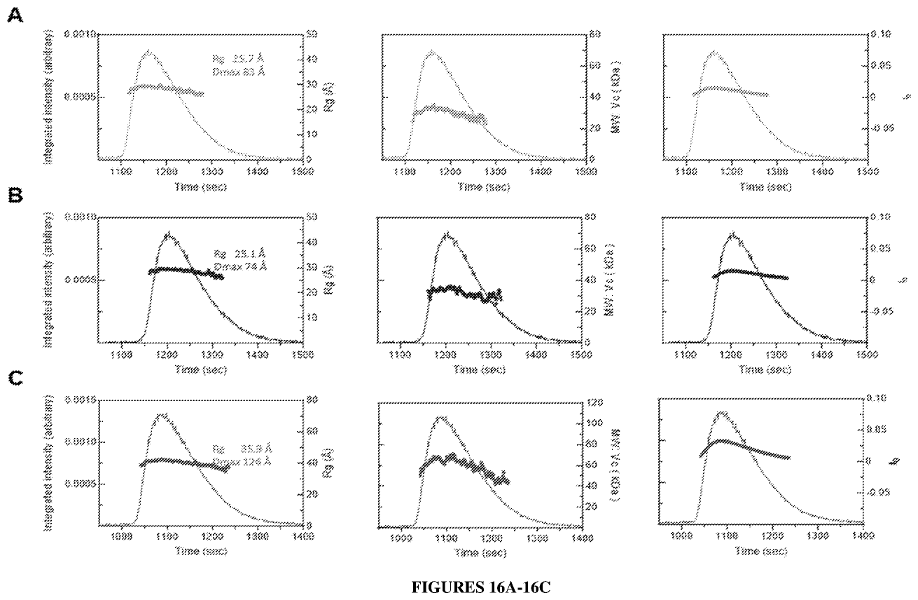

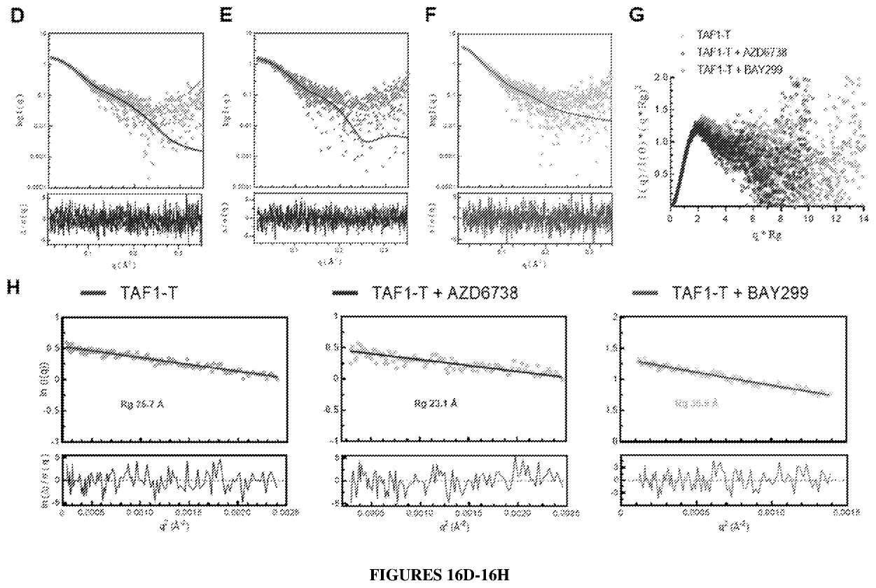

TAF1 inhibitors stabilize distinct conformational states of TAF1-T in solution: To probe structural changes of TAF1-T upon interaction with small molecule inhibitors in solution, we applied size-exclusion chromatography coupled small-angle X-ray scattering (SEC-SAXS) studies. SEC-SAXS is uniquely suited to experimentally determine conformational states of proteins through quantitative measurements of radius of gyration and particle shape. Data sets were collected for TAF1-T in the absence and presence of AZD6738 or BAY299. AZD3147 and bromosporine were excluded because of poor aqueous solubility in the SEC-SAXS buffers. Consistent measurements of macromolecule parameters across the conducted SEC-SAXS data collection frames indicated homogenous and monodisperse protein samples (FIG. 16). SEC elution and SAXS scattering profiles showed that TAF1-T alone and in the presence of AZD6738 is monomeric, whereas TAF1-T with BAY299 is dimeric (FIG. 10A-D). The scattering profile of the TAF1-T/AZD6738 complex differed from that of unliganded TAF1-T. A decrease in radius of gyration, differences in peak characteristics, pair distance distribution and globular characteristics indicated a more compact state upon interaction with AZD6738.

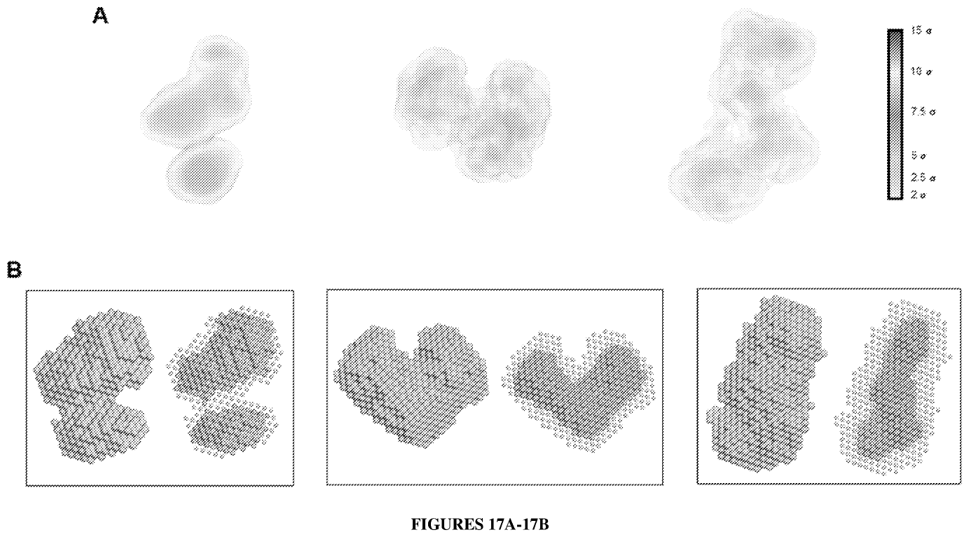

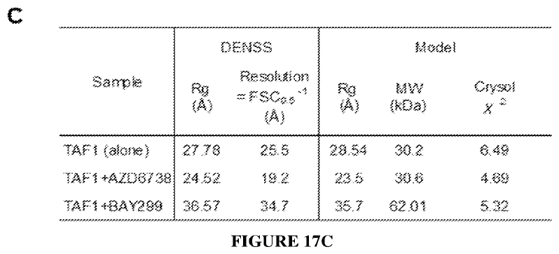

Ab initio low-resolution envelopes of unliganded and liganded TAF1-T were reconstructed from the scattering data, and models were built using rigid body fitting of isolated BD1 and BD2 (FIG. 17). The resulting model of unliganded TAF1-T showed an “open” conformation not found in any crystal structure (FIG. 10E). In this state, the two BRDs are positioned far from each other and oriented in opposite directions. By contrast, BD1 and BD2 reorient and approach each other in the model of the TAF1-T/AZD6738 complex. This conformation is similar to the “semi-closed” state observed in the crystal structure with AZD3147 (FIG. 4D-E). The SAXS data support the notion that AZD6738 stabilizes a more compact state of TAF1-T in solution. Differences in the compactness of the models derived from SAXS and crystallographic data may be caused by differences in the composition and properties of buffer vs. buffer+precipitant, and the molar ratios of protein/inhibitor during the experiment (0.05 mM/0.25 mM for SEC/SAXS, 0.3 mM/1 mM for co-crystallization).

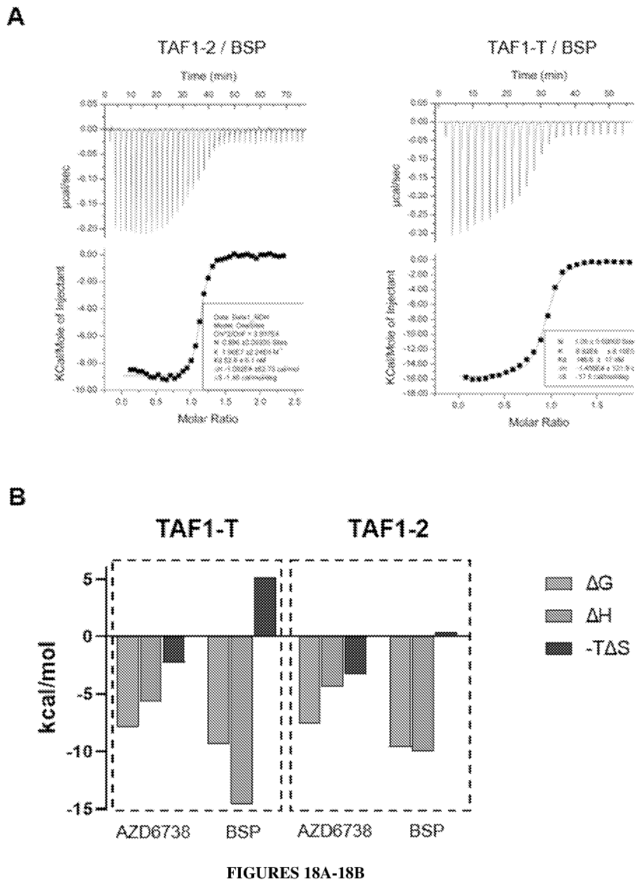

The model generated from the SAXS data of TAF1-T with BAY299 is consistent with a dimer comprised of a “closed” and an “open” state (FIG. 10E). Unfortunately, TAF1-T resisted co-crystallization attempts with BAY299 to determine the dimeric state at high resolution. ITC experiments of BAY299 with isolated BD1 and BD2 showed Kd values of 4500 and 47 nM with stoichiometries of 1.3 and 0.96, respectively (FIG. 10F). By contrast, the isotherm of BAY299 interaction with TAF1-T followed a biphasic binding curve in which the first phase of high affinity binding (Kd(1)=9.3 nM) occurred with a stoichiometry of N=˜3 and the second phase of weaker affinity binding (Kd(2)=2100 nM) with a stoichiometry of N=˜1. This finding supports the notion of dimeric TAF1-T with four binding sites for BAY299, perhaps interacting in a cooperative fashion. Thermodynamic signature analysis indicated that the first phase of interaction is mostly enthalpy-driven, while the second phase is largely entropy-driven (FIG. 10G). Binding of BAY299 to isolated BD2 was predominantly enthalpy-driven, while the interaction with BD1 was characterized by favorable entropy and enthalpy contributions. The only other TAF1 inhibitor reported to interact with both BD1 and BD2 is bromosporine (Kd=˜17 nM for TAF1-2 and Kd=˜5000 nM for TAF1-1). In our ITC studies, the interaction of bromosporine with TAF1-T was monophasic with a Kd value of 147 nM, a stoichiometry value of 1.1, and a large contribution of unfavorable entropy not seen with any of the other TAF1 inhibitors probed (FIG. 18).

Inhibition of TAF1 induces DNA damage signaling response and activates p53: Previously, it was demonstrated that a defective temperature-sensitive mutant form of TAF1 led to ATR activation, p53 phosphorylation at Ser15 and cell cycle arrest. To investigate the cellular effects of TAF1 BRD inhibition by small molecules, A549 lung cancer cells were treated with inhibitors and analyzed for p53 pathway markers by immunoblotting. The ATR inhibitor BAY1895344, which is devoid of TAF1 activity, and AZ20 induced a minor increase in p53 levels but no induction of the p53 targets p21 and MDM2 (FIG. 5A, lanes 3 and 6). The potent TAF1 inhibitor BAY299 strongly induced p53 and p21 levels comparable to those caused by the MDM2 inhibitor Nutlin (FIG. 5A, lane 5). BAY299 cooperated with Nutlin to further increase p53 accumulation and p21 expression (FIG. 5A, lane 9).

AZD6738 caused a modest increase of p53 and p21, consistent with its moderate inhibition of TAF1 (FIG. 5A, lane 4). BAY299 also caused downregulation of MDMX (FIG. 5A, lane 5), suggesting that inhibition of TAF1 BRD activates DDR. Analysis of the DDR marker pSer15 in p53 showed strong induction of phosphorylation upon BAY299 treatment of A549 cells (FIG. 5B, lane 9). AZD6738 had a modest effect on MDMX levels (FIG. 5B, lane 8), presumably because of its limited potency against TAF1 or concurrent inhibition of ATR interfering with MDMX downregulation. DNA-damaging treatment by ionizing radiation (IR) did not further enhance BAY299-mediated p53 activation (FIG. 5B, lane 5 vs 9), supporting the notion that both IR and BAY299 activate p53 through DDR signaling. To determine whether p21 induction by TAF1 inhibitors was dependent on p53, HCT116 colon cancer cells with and without p53 were tested. BAY299 induced p53 accumulation and p21 expression in a dose-dependent fashion only in HCT116 cells expressing p53 (FIG. 5C). Although BAY299 induced significant phosphorylation of p53 S15, there was no induction of γH2AX (FIG. 5D), suggesting that TAF1 BRD inhibition activated certain aspects of DNA damage signaling that led to p53 accumulation, but did not cause actual DNA strand breaks.

Previous studies showed that TAF1 inhibitors had no significant cytotoxicity against multiple tumor cell lines when tested as a single agent. Consistent with these reports, BAY299 showed weak growth inhibitory activity against HCT116 cells (IC50>20 μM). However, BAY299 synergized with Nutlin in killing HCT116 (FIG. 6A) and A549 cells (FIG. 6B). For HCT116 cells, growth inhibition by BAY299+Nutlin was strictly dependent on the presence of p53 (FIG. 6A). BAY299 and Nutlin combination treatment of HCT116 induced significant cleavage of PARP, consistent with cell death by apoptosis (FIG. 6C). Combined, the data suggest that TAF1 BRD inhibition by small molecules elicits a DNA damage signaling response that leads to p53 activation and synergizes with MDM2 inhibitor Nutlin to induce cell death.

Discussion

Although dysregulated gene transcription is a major contributing factor in cancer, the development of drugs interfering with general transcription machineries remains a challenging task. Earlier studies established that TAF1, the largest subunit of transcription factor IID, could be targeted by small molecule inhibitors of the second BRD. However, the effect of inhibitor binding on the structure of tandem BRD and the mechanism of action of TAF1 inhibitors in cells remained underexplored. Herein is reported the discovery of AZD6738 and other methylmorpholine-containing PI3KK inhibitors as bona-fide binders of TAF1 BRD.

AZD6738 showed moderate binding potential for TAF1-T (Kd=1.7 μM), which is 425 times less than the reported activity against ATR in vitro. Because of this large therapeutic window it is unlikely that AZD6738 produces significant off-target effects in a clinical setting. However, concomitant inhibition of TAF1 and ATR or other PI3KKs may be a viable strategy to alter the transcription machinery of certain cancers. The co-crystal structures of this work provide a framework for the rational design of dual TAF1-PI3KK inhibitors with differential activities for bromodomain vs. kinase.

Crystallographic and SAXS data on unliganded and liganded TAF1-T revealed global conformational changes upon interaction with inhibitors. While dual TAF1-PI3KK inhibitors induced an open-closed transition of monomeric TAF1-T, BAY299 caused dimerization of TAF1-T. During transcription initiation, TFIID undergoes large structural rearrangements. In addition to inhibiting the histone reader function, small molecule inhibitors able to stabilize distinct conformational states of TAF1-T may hinder proper assembly and positioning of TFIID in the pre-initiation complex thus affecting transcription.

Previously, it was reported that inactivating the HAT domain of TAF1 in a temperature-sensitive mutant cell line resulted in ATR activation, p53-S15 phosphorylation and induction of p21 expression. The data herein on TAF1 inhibitors also showed p53 phosphorylation and transcriptional activation, suggesting that the “reader” properties of the BRD module are equally important for the functionality of TAF1. Accordingly, disruption of either function leads to activation of DNA damage signaling and p53 phosphorylation. Interestingly, despite significant induction of p53-S15 phosphorylation (a target of ATR, ATM, DNA-PK), there was negligible induction of γH2AX (also a target of ATR and ATM). It appears that TAF1 BRD inhibition activates certain aspects of DNA damage signaling sufficient to trigger a p53 response without causing actual DNA strand breaks. Therefore, TAF1 BRD inhibitors are uniquely suited for combination treatment with MDM2 inhibitors in cancers that retain expression of wild type p53 without the genotoxicity to normal tissues and efficacy.

Materials and Methods

Reagents and compounds. Reagents and compounds for biochemical and crystallographic experiments were purchased from Sigma-Aldrich and Hampton Research unless otherwise indicated. The L1200 kinase inhibitor library was from Selleck Chemicals. AZD6738 (99% purity), AZ20 (99% purity), BI2536 (99% purity), BAY1895344 (99% purity), bromosporine (99% purity) and Nutlin-3 (>95% purity) were from Selleck Chemicals, AZD3147 (>98% purity) was from Tocris Bioscience, and BAY299 (>98% purity) was from Cayman Chemicals. Concentration of purified protein samples was determined by A280 molar absorbance using a ND-2000c spectrophotometer (Nanodrop Technologies).

Protein expression and purification. Expression plasmids for human TAF1 (Uniprot ID P21675) BD2 (residues 1501-1635) and tandem bromodomain (residues 1373-1635) were from Addgene (plasmids 39117 and 39118, respectively). The DNA sequence encoding human TAF1 BD1 (residues 1373-1499) was cloned in-frame of a modified pET28a vector providing an N-terminal hexa-histidine tag followed by a Tobacco Etch Virus (TEV) cleavage site. Plasmids were transformed into E. coli BL21 (DE3) cells and grown at 37° C. in LB medium (Fisher Scientific) containing carbenicillin (0.1 mg/mL). At OD600 of 0.6, the culture was cooled down to 18° C. and induced with 0.1 mM IPTG. After 18 h growth, the culture was harvested by centrifugation at 6,000×g for 25 min and stored at −80° C. Harvested cell pellets were resuspended in 50 mM Na/K phosphate buffer (pH 7.4) containing 150 mM NaCl, 40 mM imidazole, 0.01% w/v lysozyme and 0.01% v/v Triton X-100 at 4° C. for 1 h, subjected to sonication and the lysate was clarified by centrifugation (30,000×g for 45 min at 4° C.). Proteins were purified by FPLC at 4° C. using columns and chromatography materials from GE Healthcare. The lysate was subjected to an immobilized Ni2+ affinity chromatography column equilibrated with 50 mM Na/K phosphate buffer (pH 7.4) containing 150 mM NaCl and 40 mM imidazole using a gradient from 40 to 500 mM of imidazole. Fractions containing the target protein were combined and incubated for 2-16 h with TEV protease at 4° C., and the cleaved His6-tag was removed by a second Ni2+ affinity column run. Protein was purified to homogeneity by size exclusion chromatography using Superdex 75. The elution buffers were 20 mM HEPES/150 mM NaCl/1 mM DTT (pH 7.5) for TAF1-2, 50 mM HEPES/3000 mM NaCl/1 mM DTT (pH 7.5) for TAF1-1, and 50 mM HEPES/2 mM DTT (pH 7.5) for TAF1-T. All proteins eluted as monomers and were of crystallization grade quality (>95% purity as judged by SDS-PAGE). Peak fractions were combined, concentrated to 15-20 mg/ml and aliquots were flash-frozen in liquid N2 and stored at −80° C.

Differential scanning fluorimetry (DSF). Compound library screening (418 kinase inhibitors, Selleck Chemicals) against TAF1-T was performed by DSF essentially as described. Experiments were carried out in Applied Biosystem QuantStudio 6 Flex (hit identification) and StepOnePlus (thermal shift determination) real-time PCR system (Thermo Fisher Scientific) using sealed 384-well or 96-well plates, assayed in quadruplicate (4-4.5 μM protein, 5-5.5× of SYPRO Orange (Invitrogen). For library screening, dilutions of compound in assay buffer (50 mM HEPES pH 7.5, 150 mM NaCl, 2 mM DTT, 1% DMSO) were prepared using a Mosquito liquid dispenser (TTP Labtech Ltd). Protein in assay buffer including fluorescence dye was mixed with 100 μM compound and 2% DMSO in 20 μL reaction volumes. Reaction mixtures were heated from 25° C. to 95° C. at 1° C./min with fluorescence readings every 0.5° C. at 610 nm. The observed thermal shift (ΔTm) was recorded as the difference between the Tm of sample and DMSO reference wells.

Isothermal titration calorimetry (ITC). All experiments were conducted using an ITC200 microcalorimeter from Malvern Panalytical (Spectris PLC). BRDs were buffer exchanged using PD10 columns (GE life sciences) before the experiment and concentrated to ˜10 mg/ml (Table 4). Experiments were carried out in ITC buffer while stirring at 750 rpm in reverse titration method. The microsyringe (40 μL load volume) was loaded with protein sample (250-350 μM protein in ITC buffer) and was inserted into the calorimetric cell (0.2 ml cell volume) consisting of compound (20-25 μM in ITC buffer). All titrations were conducted using an initial control injection of 0.3 μl followed by 25-38 identical injections (1.52 or 1 μl per injection) with a duration of 3.04 or 2 sec (per injection) and a spacing of 150 sec between injections. The ratio of protein/compound was optimized to ensure complete saturation of the titrant before the final injection to ensure proper baseline determination. Experimental data were corrected for dilution and analyzed using the MicroCal™ Origin software to determine the enthalpies of binding (ΔH) and binding constants (Ka, Kd) as previously described (Wiseman et. al). Thermodynamic parameters were calculated using the basic equation of thermodynamics (ΔG=ΔH−TΔS=−RTlnKB, where ΔG, ΔH and ΔS are the changes in free energy, enthalpy and entropy of binding, respectively. A single binding site model was used for all ligands except for BAY299 interaction with TAF1-T, for which a sequential two site model was applied. Dissociation constants and thermodynamic parameters are shown in Table 4.

Microscale thermophoresis (MST). His6-TAF1 BD2 was labeled using the RED-NHS kit (NanoTemper Technologies) according to the manufacturer's instructions. Experiments were performed on the Monolith™ NT.115Pico instrument using the RED detector. For labeling, a protein concentration of 20 nM and a buffer containing 50 mM HEPES pH7.5, 150 mM NaCl, 0.05% Tween20 and 0.5 mM TCEP were used. After 30 minutes incubation with labeling reagent, the protein-label mix was centrifuged for 10 min at 20,000 rpm and then transferred to a new tube leaving 20 μL of solution. A three-fold 15-point dilution series of compound was prepared starting at 500 μM in buffer including 5% v/v DMSO. Labeled TAF1-2 was added to the compound dilution tubes and after 5-10 min incubation, the protein and compound sample were centrifuged and loaded into capillaries. Experiments were performed using LED power of 20% or 40%, and MST power of 40% or 80% with thermophoresis occurring over 30 seconds.

Kd values were determined with the MO.Affinity Analysis Software v2.1 from Nanotemper and plotted with Graphpad Prism software.

Crystallization and X-ray crystallography. All crystallization experiments were performed at 18° C. Aliquots of purified BRDs were set up for crystallization using mosquito crystallization robot (TP Labtech). Initially, coarse screens were set up using Greiner 3-well plates at three different concentrations of precipitant to protein (200+400 nl, 300+300 nl, 400+200 nl) per condition. Conditions producing crystals were further optimized and scaled up to 2 μl droplets. For co-crystallization, compound was either pre-mixed with protein on ice or added to protein-precipitant mixtures to achieve a final concentration of 0.5-2.5 mM compound in 5-10% DMSO. Crystals were cryoprotected using the well solution supplemented with ethylene glycol (15-30%) and flash frozen in liquid nitrogen. X-ray diffraction data were collected at −180° C. in-house using CuKα X-rays generated by a Rigaku Micro-Max 007-HF X-ray generator, at the beamlines 22-ID and 22-BM (SER-CAT) and at beamline 23-ID (GM/CA) of the Advanced Photon Source, Argonne National Laboratories. Data were reduced and scaled with XDS or DIALS and Aimless. Structure solution and refinement was carried out with PHENIX and model building with Coot. The structures were solved by molecular replacement using PDB entry 3UV4 as search model for TAF1-2 and 3UV5 for TAF1-T. Initial models for the small molecule ligands were generated using MarvinSketch (ChemAxon, Cambridge, MA) with ligand restraints from eLBOW of the PHENIX suite. All structures were validated by MolProbity. Figures were prepared using PyMOL (Schradinger, LLC). Data collection and refinement statistics are not shown. The coordinates and structure factors were deposited with the PDB.

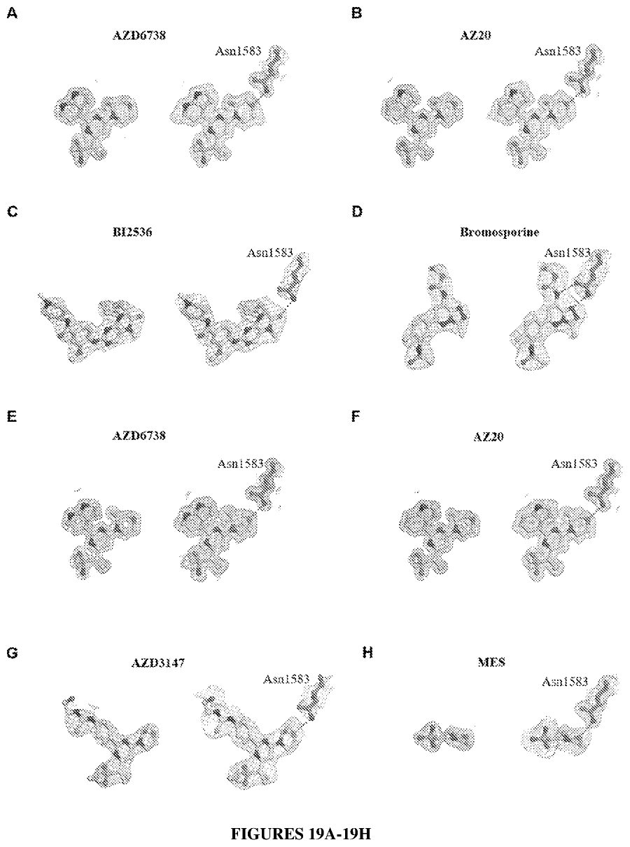

SEC-SAXS data collection and shape model construction. SAXS data collection was performed at BioCAT beamline 18ID of the Advanced Photon Source with in-line size exclusion chromatography (SEC-SAXS). Experiments were conducted at room temperature in 50 mM HEPES (pH7.5), 5% v/v ethylene glycol, 2.5% v/v DMSO and 2 mM DTT. To avoid protein aggregation and ensure ligand saturation, protein sample was loaded at 6 mg/mL (<200 μM) for estimated maximum concentration of 50 μM in the eluate. The concentration of AZD6738 and BAY299 was 250 and 13.3 μM, respectively. 300 μL of sample was loaded onto a Superdex 200 Increase 10/300 column (GE Life Sciences) and eluted with a flow rate of 0.7 mL/min. Once the SEC elute reached the SAXS quartz capillary flow cell, the sample was exposed to Synchrotron radiation. SEC column to X-ray capillary dead volume was approximately 0.1 ml. Scattering intensity was recorded on a Pilatus3 1M (Dectris) detector placed 3.5 m from the sample using a wavelength of k=1.0332 Å, which yields a momentum transfer range (scattering q-range) of 0.004<q<0.4 Å-1 (q=4π sinθ/λ, where 20 is the scattering angle). 0.5 second exposures were used with 0.5 second interval during the elution. Data were reduced using BioXTAS RAW 1.6.1. Buffer scattering was determined by averaging frames from the SEC eluent pre and post-peaks, which contained baseline levels of integrated X-ray scattering, UV absorbance and conductance. The frame intensities were corrected by subtracting the buffer contribution. The corrected frames were investigated for radius of gyration, Rg derived by the Guinier approximation I(q)=10 exp (˜q2Rg2/3) with the limits q*Rg<1.3. Final q versus I(q) data sets were generated by averaging of frames in the regions of least Rg variation, automatically selected by the BioXTAS program. Merged SAXS data with q range 0.0002-0.35 Å-1 were used to generate the forward scattering (I0), Guinier plots and radius of gyration (Rg), volumes-of-correlation (Vc), Porod's corrected volume (Vp), pair distribution function P(r), and normalized Kratky plots. The P(r) functions were further assessed and computed with the program GNOM. Molecular masses (Vc and Vp) were computed according to published methods. Ab initio shape models of TAF1-T were generated using DENSS for electron density map generation and bead modeling by DAMMIN. Multiple runs were performed to generate 20 starting models, and the final ab initio shape was reconstructed by averaging and refining the starting models. The low resolution electron density maps generated by and the bead models computed by DAMFILT of the ATSAS suit are shown in FIG. 19. Isolated models of BD1 and BD2 omitting flexible regions of the N- and C-termini (1371-1376 and 1631-1635) served for rigid body fitting into the electron density maps using PHENIX (dock_in_map) and Chimera. The hinge region connecting the two domains was modelled using ModLoop. Iterative refinement cycles were applied using Crysol 3.0 to achieve models that satisfied the experimental data (FIG. 17).

Cell culture and p53 pathway biomarker analysis. A549, HCT116, HCT116-p53−/− cells were maintained in Dulbecco modified Eagle medium with 10% fetal bovine serum and tested negative for mycoplasma contamination. A549 was obtained from the ATCC. HCT116 cell lines were kindly provided by Dr. Bert Vogelstein (Johns Hopkins Medical School). For immunoblotting, cells were lysed in RIPA buffer (25 mM Tris-HCl pH 7.4, 150 mM NaCl, 0.1% SDS, 1% NP40, 0.5% sodium deoxycholate, 1× protease inhibitor cocktail), sonicated briefly to eliminate viscosity, and centrifuged for 10 min at 14,000×g to remove insoluble debris. Cell lysate (10-50 μg of protein) was fractionated by SDS-PAGE and transferred to Immobilion P filters (Millipore). The filter was blocked for 1 h with phosphate-buffered saline containing 5% nonfat dry milk and 0.1% Tween20, incubated with primary and secondary antibodies and developed using the Supersignal reagent (Thermo Scientific). MDM2 was detected using monoclonal antibody 3G9 and MDMX was detected with monoclonal antibody 8C6 produced in house. The following antibodies were purchased from commercial sources: Actin (Sigma, A5441), p53 DO-1 (BD Pharmingen, 554293), p53 phospho-Ser15 (Cell Signaling, 9284), p21 (BD Pharmingen, 556430), H2AX and γH2AX (Millipore, #05-636, #07-627), PARP (Fisher, #BDB556362). Cell viability analysis was performed using Celltiter 96 AQ One Solution reagent following manufacturer instructions (Fisher, #PR-G3580).

Data availability and accession codes: Atomic coordinates and structure factors for the reported crystal structures have been deposited in the Protein Data Bank (www.rcsb.org) with accession codes: 7K30 (TAF1-2), 7JSP (TAF1-2/AZD6738), 7JJG (TAF1-2/AZ20), 7KOU (TAF1-2/BI2536); 7K1P (TAF1-2/bromosporine), 7K42 (TAF1-2/dioxane), 7JJH (TAF1-T), 7K03 (TAF1-T/AZD6738), 7K27 (TAF1-T/AZ20), 7K0D (TAF1-T/AZD3147), and 7K6F (TAF1-T/MES). SAXS data were deposited in the Small Angle Scattering Biological Data Bank (www.sasbdb.org).

### Example 3: Synthesis of TAF1 Inhibitors

General Schemes of the TAF1 Inhibitors are provided.

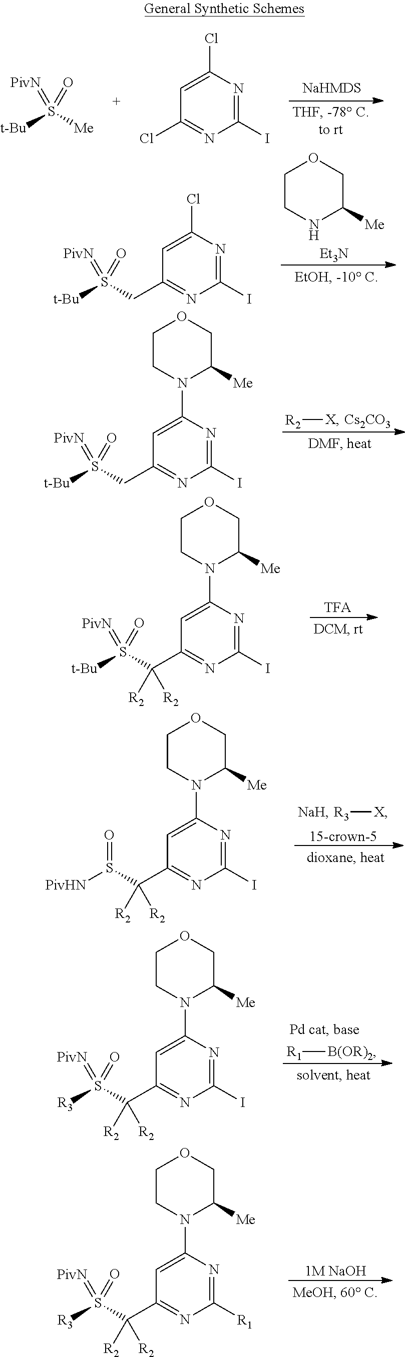

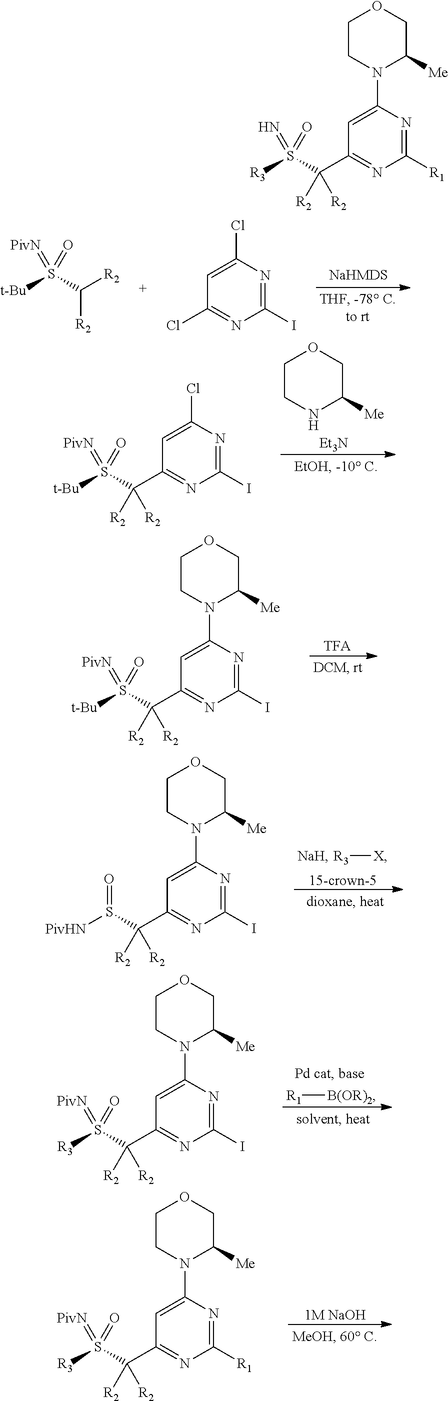

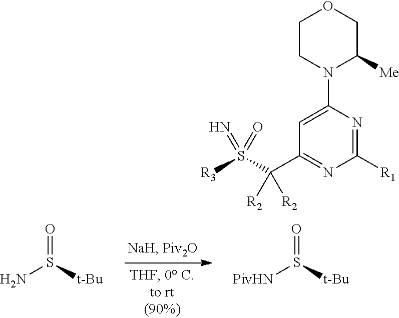

In a flame dried 1 L round-bottomed flask equipped with a stir bar, septum capped addition funnel and argon balloon was added (S)-2-methylpropane-2-sulfinamide (10.16 g, 83.8 mmol, 1.1 eq.) and THF (400 mL) then cooled to 0° C. Once cool, NaH (7.98 g, 199 mmol, 60% wt, 2.5 eq.) was added and stirred at 0° C. for 15 minutes before warming to room temperature. A solution of Piv₂O (14.9 g, 16.2 mL, 79.8 mmol, 1 eq.) in THF (250 mL) was added dropwise over 75 minutes at room temperature. Once addition was complete, the reaction stirred for an additional 60 minutes before quenching with MeOH (40 mL) followed by brine (200 mL) and saturated aqueous 1M NaOH (100 mL). The aqueous layer was extracted with EtOAc (4×200 mL), combined organic layers were washed with saturated aqueous NaHCO₃ (150 mL) and brine (150 mL), dried over Na₂SO₄, filtered and concentrated. The crude material was adsorbed to silica gel and purified by silica gel column chromatography using DCM/EtOAc (5:1 isocratic) to give (S)—N-(tert-butylsulfinyl)pivalamide (14.8 g, 72.1 mmol, 90% yield) as a white solid.

TLC: R_{f}=0.5 (DCM/EtOAC, 5:1, p-anisaldehyde stain).

¹H NMR: (500 MHz, CDCl₃) δ 7.31 (s, 1H), 1.24 (s, 9H), 1.23 (s, 9H) ppm.

¹³C NMR: (126 MHz, CDCl₃) δ 178.84, 57.40, 39.99, 27.22, 22.06 ppm.

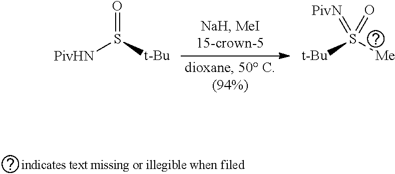

In a 500 mL round-bottomed flask equipped with a stir bar and argon balloon was added (S)—N-(tert-butylsulfinyl)pivalamide (5.05 g, 24.6 mmol, 1 eq.) and dioxane (160 mL). 15-crown-5 ether (5.86 mmol, 29.5 mmol, 1.2 eq.) was added followed by portion-wise addition of NaH (1.18 g, 29.5 mmol, 60% wt, 1.2 req.). The reaction mixture stirred at room temperature for 1 hour. Methyl iodide (Mel) (3.06 mL, 49.2 mmol, 2 eq.) was added via syringe and the reaction was heated to 50° C. for 24 hours. The reaction was cooled to room temperature and quenched with saturated aqueous NH₄Cl (150 mL). The aqueous layer was extracted with EtOAc (4×150 mL), combined organic layers were dried over Na₂SO₄, filtered and concentrated to give a crude oil. Further purification using a silica plug eluting with hexanes (200 mL) followed by 20% EtOAc in hexanes (800 mL) provided (S)—N-(tert-butyl(methyl)(oxo)-λ⁶-sulfaneylidene)pivalamide (5.06 g, 23.1 mmol, 94% yield) as a white solid.

TLC: R_{f}=0.32 (hexanes/EtOAc, 50% EtOAc, p-anisaldehyde stain).

¹H NMR: (500 MHz, CDCl₃) δ 3.23 (s, 3H), 1.46 (s, 9H), 1.16 (s, 9H) ppm.

¹³C NMR: (126 MHz, CDCl₃) δ 188.60, 60.14, 41.68, 32.04, 27.76, 22.97 ppm.

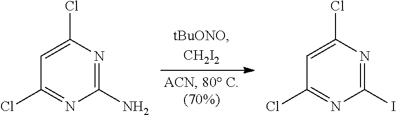 [SMILES: Nc1nc(Cl)cc(Cl)n1 ; Clc1cc(Cl)nc(I)n1]

In a 500 mL round-bottomed flask equipped with a stir bar, septum capped reflux condenser and argon balloon were added 4,6-dichloropyrimidin-2-amine (10 g, 61 mmol, 1 eq.), ACN (86 mL) and CH₂I₂ (5.41 mL, 67.1 mmol, 1.1 eq.). To the stirring solution, t-BuONO (40.3 mL, 305 mmol, 5 eq., 90%) was added via syringe and resulting reaction mixture was heated to 80° C. for 4 hours. The reaction was cooled to room temperature and the solvents were removed under vacuum. The crude residue was taken up in EtOAc (450 mL) and washed with saturated aqueous Na₂SO₃ (3×150 mL). The combined aqueous layers were back extracted with EtOAc (2×150 mL) and combined with the first organic layer. The combined organic layers were dried over Na₂SO₄ then filtered and adsorbed to silica gel. Further purification by silica gel column chromatography using hexanes and EtOAc (0% to 10% EtOAc) provided 4,6-dichloro-2-iodopyrimidine (11.7 g, 42.7 mmol, 70% yield) as an off-white solid.

TLC: R_{f}=0.58 (10% EtOAc in hexanes)

¹H NMR: (500 MHz, CDCl₃) δ 7.39 (s, 1H) ppm.

¹³C NMR: (126 MHz, CDCl₃) δ 161.04, 125.97, 120.87 ppm.

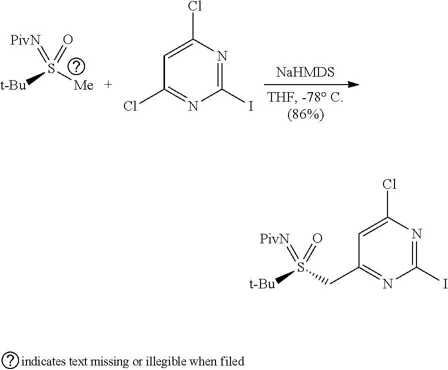

In a 200 mL round-bottomed flask equipped with a stir bar was added (S)—N-(tert-butyl(methyl)(oxo)-λ6-sulfaneylidene)pivalamide (1.62 g, 7.39 mmol, 1 eq.), 4,6-dichloro-2-iodopyrimidine (2.03 g, 7.39 mmol, 1 eq.) and THF (74 mL) then cooled to −78° C. NaHMDS (8.12 mL, 16.25 mmol, 2.2 eq., 1M in THF) was added dropwise. The resulting solution stirred at −78° C. for 1.5 hours then removed from the dry ice bath and quenched with saturated aqueous NH₄Cl (80 mL). The aqueous layer was extracted with EtOAc (4×70 mL), dried over Na₂SO₄, filtered and concentrated. Further purification by silica gel column chromatography using hexanes/EtOAc (0% to 20% EtOAc) provided (S)—N-(tert-butyl((6-chloro-2-iodopyrimidin-4-yl)methyl)(oxo)-λ⁶-sulfaneylidene)pivalamide (2.92 g, 6.38 mmol, 86% yield) as a pale yellow solid.

TLC: R_{f}=0.7 (40% EtOAc, UV)

¹H NMR: (500 MHz, CDCl₃) δ 7.85 (s, 1H), 5.04 (d, J=13.6 Hz, 1H), 4.70 (d, J=13.6 Hz, 1H), 1.49 (s, 9H), 1.19 (s, 9H) ppm.

¹³C NMR: (126 MHz, CDCl₃) δ 189.33, 161.49, 161.22, 126.54, 123.72, 64.00, 52.09, 41.85, 27.63, 23.35 ppm.

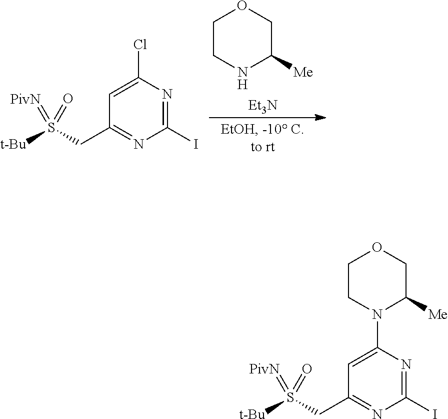

In a 20 mL septum capped vial equipped with a stir bar was added (S)—N-(tert-butyl((6-chloro-2-iodopyrimidin-4-yl)methyl)(oxo)-λ⁶-sulfaneylidene)pivalamide (0.344 g, 0.751 mmol, 1 eq.) and EtOH (7.5 mL) then cooled to −10° C. Once cool, (R)-3-methylmorpholine (0.076 g, 0.751 mmol, 1 eq.) was added followed by dropwise addition of Et₃N (0.147 mL, 0.106 g, 1.05 mmol, 1.4 eq). The resulting mixture was stirred at −10° C. for 5 hours then warmed to room temperature where it stirred for 19 hours. Nearly full consumption of (S)—N-(tert-butyl((6-chloro-2-iodopyrimidin-4-yl)methyl)(oxo)-λ⁶-sulfaneylidene)pivalamide was observed by LC-MS. A second portion of (R)-3-methylmorpholine (0.015 g, 0.15 mmol, 0.2 eq.) was added and continued stirring at room temperature for 6 hours at which time full consumption was observed (LC-MS). The solvent was removed under reduced pressure and the crude oil was taken up in EtOAc (30 mL) and saturated aqueous NH₄Cl (30 mL). The aqueous layer was extracted with EtOAc (4×20 mL), dried over Na₂SO₄, filtered and concentrated. The crude material containing N—((S)-tert-butyl((2-iodo-6-((R)-3-methylmorpholino)pyrimidin-4-yl)methyl)(oxo)-λ6-sulfaneylidene)pivalamide was used in the next step without further purification or characterization.

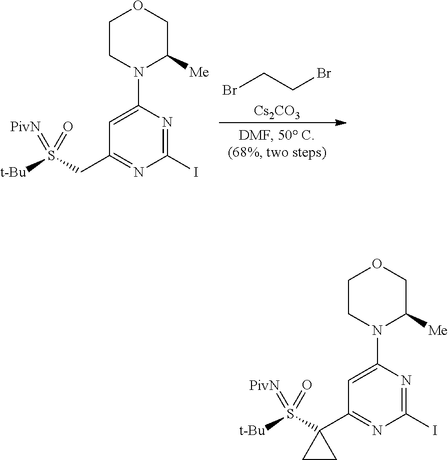

In a 20 mL septum capped vial equipped with a stir bar was added crude N—((S)-tert-butyl((2-iodo-6-((R)-3-methylmorpholino)pyrimidin-4-yl)methyl)(oxo)-λ⁶-sulfaneylidene)pivalamide (0.392 g, 0.751 mmol, 1 eq.) and DMF (7.5 mL). 1,2-dibromoethane (0.325 mL, 3.76 mmol, 5 eq.) and CsCO₃ (0.734 g, 2.25 mmol, 3 eq.) were added then heated to 50° C. for 38 hours. The reaction was quenched with saturated aqueous NH₄Cl (30 mL) and the aqueous layer was extracted with EtOAc (4×25 mL). Combined organic layers were dried over Na₂SO₃, filtered and concentrated. Further purification by silica gel column using hexanes/EtOAc (0% to 40% EtOAc) to provide N—((R)-tert-butyl(1-(2-iodo-6-((R)-3-methylmorpholino)pyrimidin-4-yl)cyclopropyl)(oxo)-λ⁶-sulfaneylidene)pivalamide (0.280 g, 0.511 mmol, 68% yield) as a yellow oil.

TLC: R_{f}=0.47 (40% EtOAc, UV)

¹H NMR: (500 MHz, CDCl₃) δ 6.81 (s, 1H), 4.34-4.19 (m, 1H), 3.89 (dd, J=11.7, 3.7 Hz, 1H), 3.85-3.69 (m, 1H), 3.67 (d, J=11.6 Hz, 1H), 3.56 (dd, J=11.7, 2.6 Hz, 1H), 3.39 (ddd, J=12.3, 11.5, 3.0 Hz, 1H), 3.18 (ddd, J=13.5, 12.3, 3.8 Hz, 1H), 2.27-2.16 (m, 1H), 1.72 (ddd, J=10.5, 7.7, 5.6 Hz, 1H), 1.66-1.60 (m, 1H), 1.39 (s, 9H), 1.34-1.27 (m, 1H), 1.21 (d, J=6.7 Hz, 3H), 1.09 (s, 9H) ppm.

¹³C NMR: (126 MHz, CDCl₃) δ 187.35, 163.32, 160.88, 128.46, 105.15, 70.72, 66.90, 66.48, 47.19, 42.74, 42.14, 39.40, 27.86, 24.59, 15.80, 14.85, 14.14 ppm.

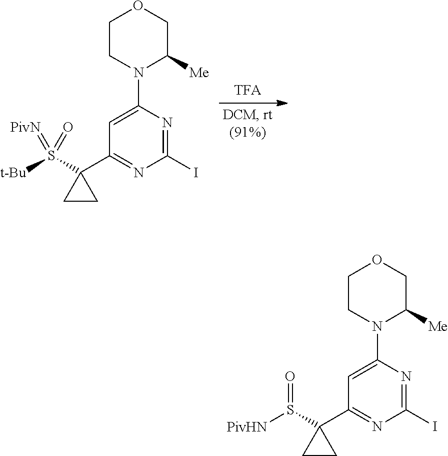

In a 100 mL round-bottomed flask equipped with a stir bar was added N—((R)-tert-butyl(1-(2-iodo-6-((R)-3-methylmorpholino)pyrimidin-4-yl)cyclopropyl)(oxo)-λ⁶-sulfaneylidene)pivalamide (1.23 g, 2.43 mmol, 1 eq.) and DCM (32 mL). TFA (0.257 mmol, 3.36 mmol, 1.5 eq) was added dropwise and the reaction stirred at room temperature for 1.5 hours. The reaction was quenched with saturated aqueous NaHCO₃ (35 mL) and the aqueous layer extracted with DCM (3×40 mL). Combined organic layers were dried over Na₂SO₄, filtered and concentrated. Further purification by silica gel column chromatography using hexanes/EtOAc (0% to 60% EtOAc) provided N—((R)-(1-(2-iodo-6-((R)-3-methylmorpholino)pyrimidin-4-yl)cyclopropyl)sulfinyl)pivalamide (1.01 g, 2.04 mmol, 91% yield) as a foam solid.

TLC: R_{f}=0.21 (60% EtOAc, UV)

¹H NMR: (500 MHz, CDCl₃) δ 7.93 (s, 1H), 6.45 (s, 1H), 4.66 (qd, J=6.8, 3.1 Hz, 1H), 4.29 (dd, J=13.7, 2.7 Hz, 1H), 3.98 (dd, J=11.5, 3.7 Hz, 1H), 3.76 (d, J=11.5 Hz, 1H), 3.63 (dd, J=11.5, 3.1 Hz, 1H), 3.48 (td, J=11.9, 3.0 Hz, 1H), 3.25 (ddd, J=13.5, 12.5, 3.8 Hz, 1H), 1.78-1.71 (m, 1H), 1.52-1.44 (m, 2H), 1.33-1.30 (m, 1H), 1.28 (d, J=6.8 Hz, 3H), 1.16 (s, 9H) ppm.

¹³C NMR: (126 MHz, CDCl₃) δ 178.42, 166.15, 162.22, 160.31, 107.53, 70.96, 66.87, 47.00, 44.77, 39.58, 39.31, 27.12, 14.06, 9.06 ppm.

### Alternative Route to N—((R)-(1-(2-iodo-6-((R)-3-methylmorpholino)pyrimidin-4-yl)cyclopropyl)sulfinyl)pivalamide

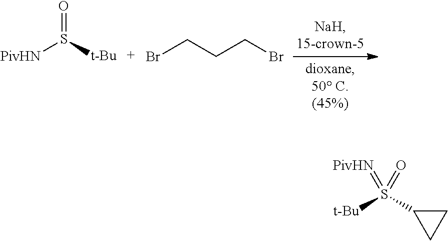

In a 1 L round-bottomed flask equipped with a stir bar and argon balloon was added (S)-2-methylpropane-2-sulfinamide (9.80 g, 47.7 mmol, 1 eq.) and dioxane (470 mL). 15-crown-5 ether (11.4 mL, 57.3 mmol, 1.2 eq.) was added followed by NaH (2.29 g, 57.28 mmol, 60% wt, 1.2 eq.) then stirred at room temperature for 20 minutes. 1,3-dibromopropane (5.84 mL, 57.28 mmol, 1.2 eq.) was added and the reaction mixture was heated to 50° C. for 20 hours. The reaction mixture was cooled to room temperature and an additional 1.2 eq. of NaH was added then heated to 50° C. for an additional 18 hours. Another 0.6 eq. of NaH was added at room temperature then heated to 50° C. for 6 hours at which time the reaction was cooled to room temperature then quenched with saturated aqueous NH₄Cl (120 mL) and brine (150 mL). The aqueous layer was extracted with EtOAc (4×150 mL), combined organic layers were washed with 1M NaOH (2×100 mL) then brine (150 mL), dried over Na₂SO₄, filtered and concentrated. Further purification by silica gel column chromatography using hexanes/EtOAc (0% to 20%) provided (S)—N-(tert-butyl(cyclopropyl)(oxo)-λ⁶-sulfaneylidene)pivalamide (5.30 g, 21.6 mmol, 45% yield) as a white solid.

TLC: R_{f}=0.36 (hexanes/EtOAc, 40% EtOAc, p-anisaldehyde stain).

¹H NMR: (500 MHz, CDCl₃) δ 2.51-2.43 (m, 1H), 1.63-1.56 (m, 1H), 1.49 (s, 8H), 1.31-1.23 (m, 1H), 1.22-1.13 (m, 10H), 1.12-1.07 (m, 1H) ppm.

¹³C NMR: (126 MHz, CDCl₃) δ 186.93, 62.82, 41.87, 27.91, 23.93, 23.86, 6.60, 5.01 ppm.

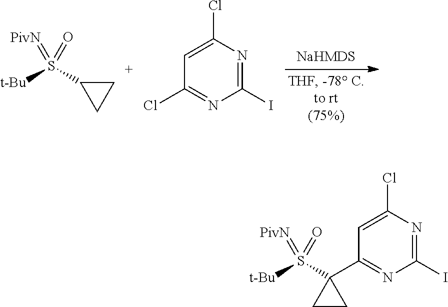

In a 200 mL round-bottomed flask equipped with a stir bar and argon balloon was added (S)—N-(tert-butyl(cyclopropyl)(oxo)-λ⁶-sulfaneylidene)pivalamide (1.0 g, 4.08 mmol, 1 eq.), 4,6-dichloro-2-iodopyrimidine (1.12 g, 4.08 mmol, 1 eq.) and THF (41 mL) then cooled to −78° C. NaHMDS (2.45 mL, 4.89 mmol, 2M, 1.2 eq.) was added dropwise and stirred at −78° C. for 2.5 hours then gradually warmed to 0° C. where the reaction was quenched with saturated aqueous NH₄Cl (60 mL). The aqueous layer was extracted with EtOAc (4×50 mL), combined organic layers dried over Na₂SO₄, filtered and concentrated. Further purification by silica gel column chromatography using hexane/EtOAc (0% to 30% EtOAc) provided (R)—N-(tert-butyl(1-(6-chloro-2-iodopyrimidin-4-yl)cyclopropyl)(oxo)-λ⁶-sulfaneylidene)pivalamide (1.48 g, 3.06 mmol, 75% yield) as a yellow oil.

TLC: R_{f}=0.48 (hexanes/EtOAC, 20% EtOAc, UV)

¹H NMR: (500 MHz, CDCl₃) δ 7.72 (s, 1H), 2.47 (ddd, J=10.6, 8.2, 6.0 Hz, 1H), 1.98 (ddd, J=9.6, 8.2, 5.7 Hz, 1H), 1.87 (ddd, J=10.6, 8.0, 5.7 Hz, 1H), 1.40 (s, 9H), 1.20 (s, 9H) ppm.

¹³C NMR: (126 MHz, CDCl₃) δ 188.03, 167.08, 161.00, 126.69, 123.60, 67.07, 42.24, 41.70, 27.73, 24.66, 17.04, 15.00 ppm.

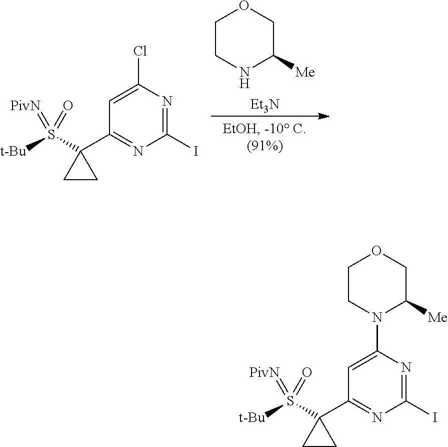

In a 100 mL round-bottomed flask equipped with a stir bar and an argon balloon was added (R)—N-(tert-butyl(1-(6-chloro-2-iodopyrimidin-4-yl)cyclopropyl)(oxo)-λ⁶-sulfaneylidene)pivalamide (1.35 g, 2.95 mmol, 1 eq.) and EtOH (50 mL) then cooled to −10° C. Et₃N (0.575 mL, 4.13 mmol, 1.4 eq.) was added followed by dropwise addition of (R)-3-methylmorpholine (0.328 g, 3.24 mmol, 1.1 eq.). The reaction mixture stirred at −10° C. for 18 hours where it warmed to room temperature. An addition 0.2 eq. of Et₃N and (R)-3-methylmorpholine were added and continue stirring at room temperature for 24 hours. Solvent was removed under reduced pressure to give crude oil that was taken up in EtOAc (75 mL) and saturated aqueous NH₄Cl (50 mL). The aqueous layer was extracted with EtOAc (4×75 mL), combined organic layers were dried over Na₂SO₄, filtered and concentrated. Further purification by silica gel column chromatography using hexanes/EtOAc (0% to 40% EtOAc) provided N—((R)-tert-butyl(1-(2-iodo-6-((R)-3-methylmorpholino)pyrimidin-4-yl)cyclopropyl)(oxo)-λ⁶-sulfaneylidene)pivalamide (1.39 g, 2.53 mmol, 91% yield) as an amorphous solid.

TLC: R_{f}=0.47 hexanes/EtOAC, 40% EtOAc, UV)

¹H NMR: (500 MHz, CDCl₃) δ 6.81 (s, 1H), 4.34-4.19 (m, 1H), 3.89 (dd, J=11.7, 3.7 Hz, 1H), 3.85-3.69 (m, 1H), 3.67 (d, J=11.6 Hz, 1H), 3.56 (dd, J=11.7, 2.6 Hz, 1H), 3.39 (ddd, J=12.3, 11.5, 3.0 Hz, 1H), 3.18 (ddd, J=13.5, 12.3, 3.8 Hz, 1H), 2.27-2.16 (m, 1H), 1.72 (ddd, J=10.5, 7.7, 5.6 Hz, 1H), 1.66-1.60 (m, 1H), 1.39 (s, 9H), 1.34-1.27 (m, 1H), 1.21 (d, J=6.7 Hz, 3H), 1.09 (s, 9H) ppm.

¹³C NMR: (126 MHz, CDCl₃) δ 187.35, 163.32, 160.88, 128.46, 105.15, 70.72, 66.90, 66.48, 47.19, 42.74, 42.14, 39.40, 27.86, 24.59, 15.80, 14.85, 14.14 ppm.

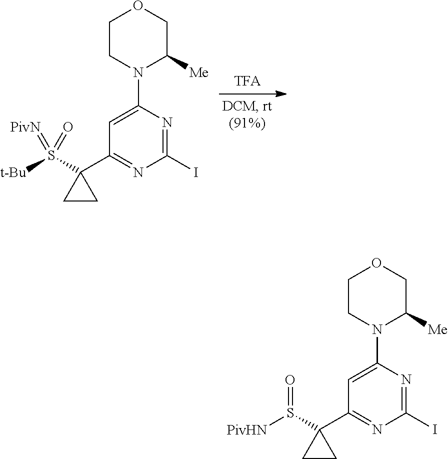

In a 100 mL round-bottomed flask equipped with a stir bar was added N—((R)-tert-butyl(1-(2-iodo-6-((R)-3-methylmorpholino)pyrimidin-4-yl)cyclopropyl)(oxo)-λ⁶-sulfaneylidene)pivalamide (1.23 g, 2.24 mmol, 1 eq.) and DCM (32 mL). TFA (0.257 mL, 3.36 mmol, 1.5 eq.) was added dropwise then stirred at room temperature for 1.5 hours. The reaction was quenched with saturated aqueous NaHCO₃ (35 mL), aqueous layer extracted with DCM (3×40 mL), combined organic layers dried over Na₂SO₄, filtered and concentrated. Further purification by silica gel column chromatography using hexanes/EtOAc (0% to 60% EtOAc) to provide N—((R)-(1-(2-iodo-6-((R)-3-methylmorpholino)pyrimidin-4-yl)cyclopropyl)sulfinyl)pivalamide (1.01 g, 2.04 mmol, 91% yield) as an amorphous solid.

TLC: R_{f}=0.21 hexanes/EtOAC, 60% EtOAc, UV)

¹H NMR: (500 MHz, CDCl₃) δ 7.93 (s, 1H), 6.45 (s, 1H), 4.66 (qd, J=6.8, 3.1 Hz, 1H), 4.29 (dd, J=13.7, 2.7 Hz, 1H), 3.98 (dd, J=11.5, 3.7 Hz, 1H), 3.76 (d, J=11.5 Hz, 1H), 3.63 (dd, J=11.5, 3.1 Hz, 1H), 3.48 (td, J=11.9, 3.0 Hz, 1H), 3.25 (ddd, J=13.5, 12.5, 3.8 Hz, 1H), 1.78-1.71 (m, 1H), 1.52-1.44 (m, 2H), 1.33-1.30 (m, 1H), 1.28 (d, J=6.8 Hz, 3H), 1.16 (s, 9H) ppm.

¹³C NMR: (126 MHz, CDCl₃) δ 178.42, 166.15, 162.22, 160.31, 107.53, 70.96, 66.87, 47.00, 44.77, 39.58, 39.31, 27.12, 14.06, 9.06 ppm.

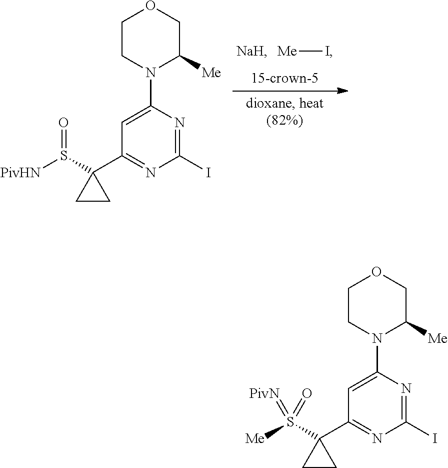

In a 20 mL septum capped vial equipped with a stir bar and argon balloon was added N—((R)-(1-(2-iodo-6-((R)-3-methylmorpholino)pyrimidin-4-yl)cyclopropyl)sulfinyl)pivalamide (0.360 g, 0.731 mmol, 1 eq.) and dioxane (7.3 mL). 15-crown-5 (0.174 mL, 0.877 mmol, 1.2 eq.) and NaH (0.035 g, 0.877 mmol, 60% wt, 1.2 eq.) were added and stirred at room temperature for 15 minutes. Mel (0.091 mL, 1.46 mmol, 2 eq.) was added then heated to 50° C. for 36 hours. The reaction mixture was cooled to room temperature and quenched with saturated aqueous NH₄Cl (15 mL) and the aqueous layer was extracted with EtOAc (4×20 mL). Combined organic layers were dried over Na₂SO₄, filtered and concentrated. Further purification by silica gel column chromatography using hexanes/EtOAc (0% to 40% EtOAc) provided N—((R)-(1-(2-iodo-6-((R)-3-methylmorpholino)pyrimidin-4-yl)cyclopropyl)(methyl)(oxo)-λ⁶-sulfaneylidene)pivalamide (0.303 g, 0.598 mmol, 82% yield) as a light-yellow oil.

TLC: R_{f}=0.36 (50% EtOAc, UV)

¹H NMR: (500 MHz, CDCl₃) δ 6.69 (s, 1H), 4.47-4.23 (m, 1H), 4.07-3.87 (m, 2H), 3.78 (d, J=11.6 Hz, 1H), 3.65 (dd, J=11.6, 3.1 Hz, 1H), 3.49 (td, J=12.0, 3.0 Hz, 1H), 3.38 (s, 3H), 3.29 (td, J=12.9, 3.7 Hz, 1H), 2.11 (ddd, J=10.5, 7.6, 6.0 Hz, 1H), 1.80 (ddd, J=10.5, 7.3, 5.5 Hz, 1H), 1.62-1.56 (m, 1H), 1.39 (ddd, J=9.5, 7.7, 5.5 Hz, 1H), 1.33 (d, J=6.8 Hz, 3H), 1.08 (s, 9H) ppm.

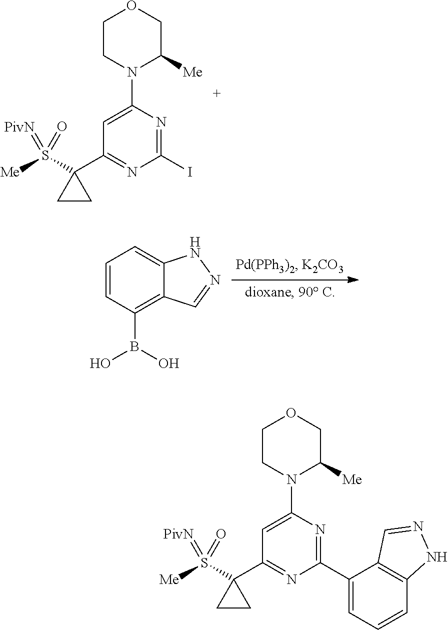

In a 2-dram septum capped reaction vial equipped with a stir bar and argon balloon was added N—((R)-(1-(2-iodo-6-((R)-3-methylmorpholino)pyrimidin-4-yl)cyclopropyl)(methyl)(oxo)-λ⁶-sulfaneylidene)pivalamide (0.078 g, 0.154 mmol, 1 eq.), (1H-indazol-4-yl)boronic acid (0.030 g, 0.184 mmol, 1.2 eq.), K₂CO₃ (0.043 g, 0.308 mmol, 2 eq.), Pd(PPh₃)₂Cl₂ (0.011 g, 0.015 mmol, 0.1 eq.) and dioxane (1.5 mL). The reaction mixture was degassed by bubbling argon through the solution for 20 minutes then heated to 90° C. for 20 hours. The reaction was cooled to room temperature, diluted with EtOAc (20 mL), filtered through a pad of celite then concentrated. The crude oil containing N—((R)-(1-(2-(1H-indazol-4-yl)-6-((R)-3-methylmorpholino)pyrimidin-4-yl)cyclopropyl)(methyl)(oxo)-λ⁶-sulfaneylidene)pivalamide was used in the next step without further purification or characterization.

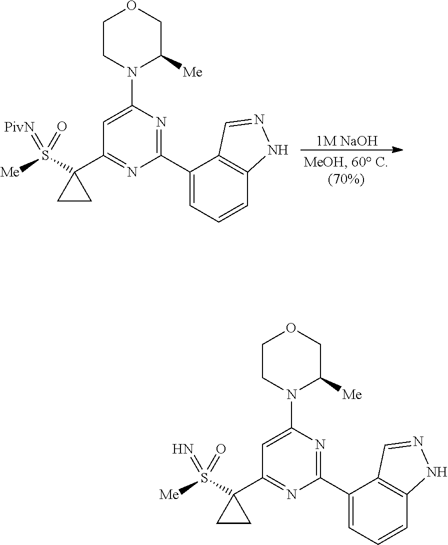

In a 2-dram septum capped reaction vial equipped with a stir bar was added crude N—((R)-(1-(2-(1H-indazol-4-yl)-6-((R)-3-methylmorpholino)pyrimidin-4-yl)cyclopropyl)(methyl)(oxo)-λ⁶-sulfaneylidene)pivalamide (0.076 g, 0.154 mmol, 1 eq.) and MeOH (1.5 mL). An aqueous solution of NaOH (1.54 mL, 1.54 mmol, 1M, 10 eq.) was added and then heated to 60° C. for 22 hours. The reaction was cooled to room temperature, diluted with EtOAc (10 mL) and H₂O (4 mL). The aqueous layer was extracted with EtOAc (4×15 mL), combined organic layers were dried over Na₂SO₄, filtered and concentrated. Further purification by silica gel column chromatography using DCM/MeOH (0% to 4% MeOH) provided (R)-(1-(2-(1H-indazol-4-yl)-6-((R)-3-methylmorpholino)pyrimidin-4-yl)cyclopropyl)(imino)(methyl)-λ⁶-sulfanone (0.045 g, 0.109 mmol, 71% yield) as a colorless amorphous solid.

TLC: R_{f}=0. (DCM/MeOH, 5% MeOH, UV)

¹H NMR: (500 MHz, CDCl₃) δ 8.93 (d, J=0.8 Hz, 1H), 8.27 (dd, J=7.3, 0.8 Hz, 1H), 7.62 (dt, J=8.3, 0.8 Hz, 1H), 7.49 (dd, J=8.3, 7.3 Hz, 1H), 6.86 (s, 1H), 4.54 (s, 1H), 4.23-4.11 (m, 1H), 4.08 (dd, J=11.5, 3.8 Hz, 1H), 3.86 (d, J=11.5 Hz, 1H), 3.77 (dd, J=11.5, 3.1 Hz, 1H), 3.63 (td, J=11.9, 3.1 Hz, 1H), 3.39 (td, J=12.8, 3.9 Hz, 1H), 3.17 (s, 3H), 1.85-1.77 (m, 2H), 1.59-1.54 (m, 2H), 1.39 (d, J=6.8 Hz, 3H) ppm.

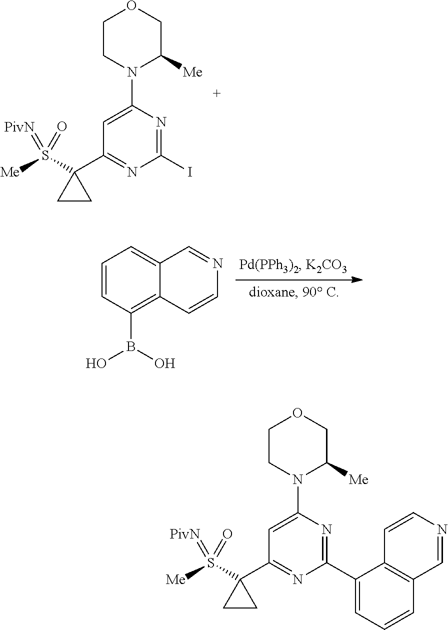

In a 2-dram septum capped reaction vial equipped with a stir bar and argon balloon was added N—((R)-(1-(2-iodo-6-((R)-3-methylmorpholino)pyrimidin-4-yl)cyclopropyl)(methyl)(oxo)-λ⁶-sulfaneylidene)pivalamide (0.100 g, 0.197 mmol, 1 eq.), isoquinolin-5-ylboronic acid (0.041 g, 0.237 mmol, 1.2 eq.), K₂CO₃ (0.055 g, 0.395 mmol, 2 eq.), Pd(PPh₃)₂Cl₂ (0.014 g, 0.020 mmol, 0.1 eq.) and dioxane (2 mL). The reaction mixture was degassed by bubbling argon through the solution for 20 minutes then heated to 90° C. for 20 hours. The reaction was cooled to room temperature, diluted with EtOAc (20 mL), filtered through a pad of celite then concentrated. The crude oil was used in the next step without further purification or characterization.

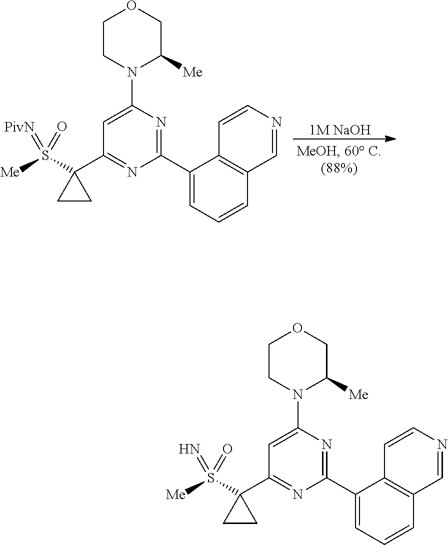

In a 2-dram septum capped reaction vial equipped with a stir bar was N—((R)-(1-(2-(isoquinolin-5-yl)-6-((R)-3-methylmorpholino)pyrimidin-4-yl)cyclopropyl)(methyl)(oxo)-λ⁶-sulfaneylidene)-pivalamide (0.100 g, 0.197 mmol, 1 eq.) and MeOH (2 mL). An aqueous solution of NaOH (1.97 mL, 1.97 mmol, 1M, 10 eq.) was added and then heated to 60° C. for 15 hours. The reaction was cooled to room temperature, diluted with EtOAc (10 mL) and H₂O (4 mL). The aqueous layer was extracted with EtOAc (4×15 mL), combined organic layers were dried over Na₂SO₄, filtered and concentrated. Further purification by silica gel column chromatography using DCM/MeOH (0% to 4% MeOH) provided (R)-imino(1-(2-(isoquinolin-5-yl)-6-((R)-3-methylmorpholino)pyrimidin-4-yl)cyclopropyl)(methyl)-λ⁶-sulfanone (0.073 g, 0.173 mmol, 88% yield) as a light-yellow oil.

¹H NMR: (500 MHz, CDCl₃) δ 9.31 (s, 1H), 8.65 (d, J=5.9 Hz, 1H), 8.56 (s, 1H), 8.33 (dd, J=7.3, 1.2 Hz, 1H), 8.07 (d, J=8.1 Hz, 1H), 7.74-7.65 (m, 1H), 6.88 (s, 1H), 4.48 (s, 1H), 4.17-4.09 (m, 1H), 4.04 (dd, J=11.5, 3.8 Hz, 1H), 3.82 (d, J=11.5 Hz, 1H), 3.74 (dd, J=11.6, 3.1 Hz, 1H), 3.60 (td, J=12.0, 3.1 Hz, 1H), 3.36 (td, J=12.9, 3.9 Hz, 1H), 3.17 (s, 3H), 1.83-1.77 (m, 2H), 1.54 (dp, J=6.3, 2.2 Hz, 2H), 1.39 (d, J=6.8 Hz, 3H) ppm.

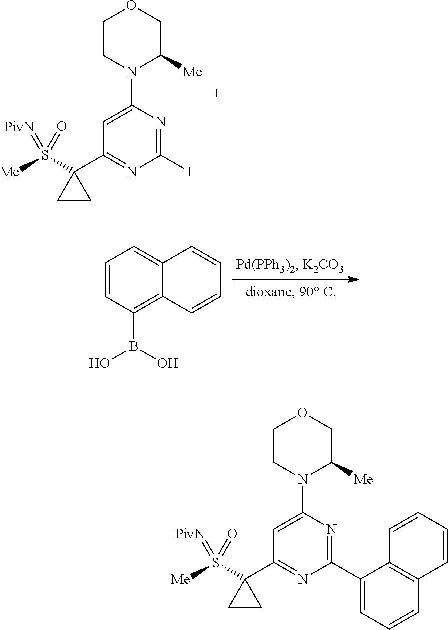

In a 2-dram septum capped reaction vial equipped with a stir bar and argon balloon was added N—((R)-(1-(2-iodo-6-((R)-3-methylmorpholino)pyrimidin-4-yl)cyclopropyl)(methyl)(oxo)-λ⁶-sulfaneylidene)pivalamide (0.100 g, 0.197 mmol, 1 eq.), naphthalen-1-ylboronic acid (0.041 g, 0.237 mmol, 1.2 eq.), K₂CO₃ (0.055 g, 0.395 mmol, 2 eq.), Pd(PPh₃)₂Cl₂ (0.007 g, 0.001 mmol, 0.05 eq.) and dioxane (2 mL). The reaction mixture was degassed by bubbling argon through the solution for 20 minutes then heated to 90° C. for 20 hours. The reaction was cooled to room temperature, diluted with EtOAc (20 mL), filtered through a pad of celite then concentrated. The crude oil was used in the next step without further purification or characterization.

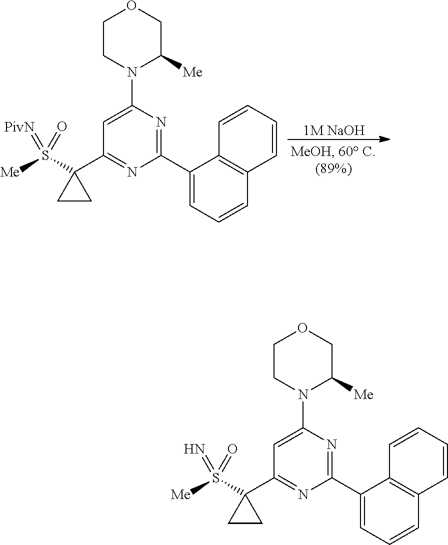

In a 2-dram septum capped reaction vial equipped with a stir bar was N—((R)-methyl(1-(6-((R)-3-methylmorpholino)-2-(naphthalen-1-yl)pyrimidin-4-yl)cyclopropyl)(oxo)-λ⁶-sulfaneylidene)-pivalamide (0.100 g, 0.197 mmol, 1 eq.) and MeOH (2 mL). An aqueous solution of NaOH (1.97 mL, 1.97 mmol, 1M, 10 eq.) was added and then heated to 60° C. for 15 hours. The reaction was cooled to room temperature, diluted with EtOAc (10 mL) and H₂O (4 mL). The aqueous layer was extracted with EtOAc (4×15 mL), combined organic layers were dried over Na₂SO₄, filtered and concentrated. Further purification by silica gel column chromatography using DCM/MeOH (0% to 4% MeOH) provided (R)-imino(methyl)(1-(6-((R)-3-methylmorpholino)-2-(naphthalen-1-yl)pyrimidin-4-yl)cyclopropyl)-λ⁶-sulfanone (0.073 g, 0.173 mmol, 89% yield) as a colorless amorphous solid.

¹H NMR: (500 MHz, CDCl₃) δ 8.61 (dd, J=6.3, 3.5 Hz, 1H), 7.97 (dd, J=7.2, 1.1 Hz, 1H), 7.93 (d, J=8.2 Hz, 1H), 7.90 (dd, J=6.2, 3.4 Hz, 1H), 7.55 (dd, J=8.1, 7.3 Hz, 1H), 7.50 (dd, J=6.4, 3.3 Hz, 2H), 6.87 (s, 1H), 4.50 (s, 1H), 4.18-4.09 (m, 1H), 4.02 (dd, J=11.5, 3.7 Hz, 1H), 3.80 (d, J=11.5 Hz, 1H), 3.73 (dd, J=11.5, 3.1 Hz, 1H), 3.59 (td, J=12.0, 3.1 Hz, 1H), 3.35 (td, J=12.9, 3.9 Hz, 1H), 3.21 (s, 3H), 1.84-1.75 (m, 2H), 1.55 (q, J=2.8 Hz, 2H), 1.38 (d, J=6.8 Hz, 3H) ppm.

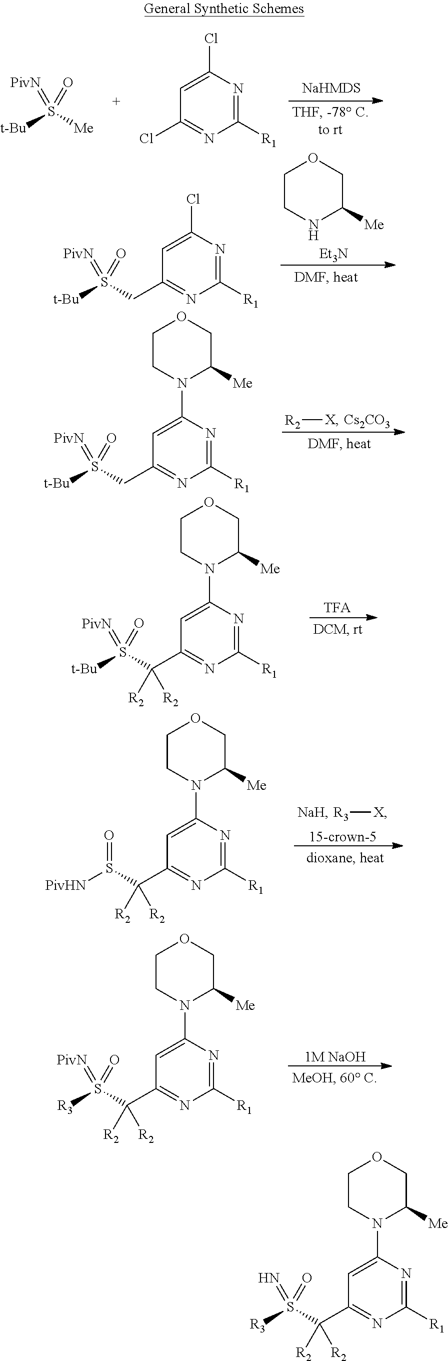

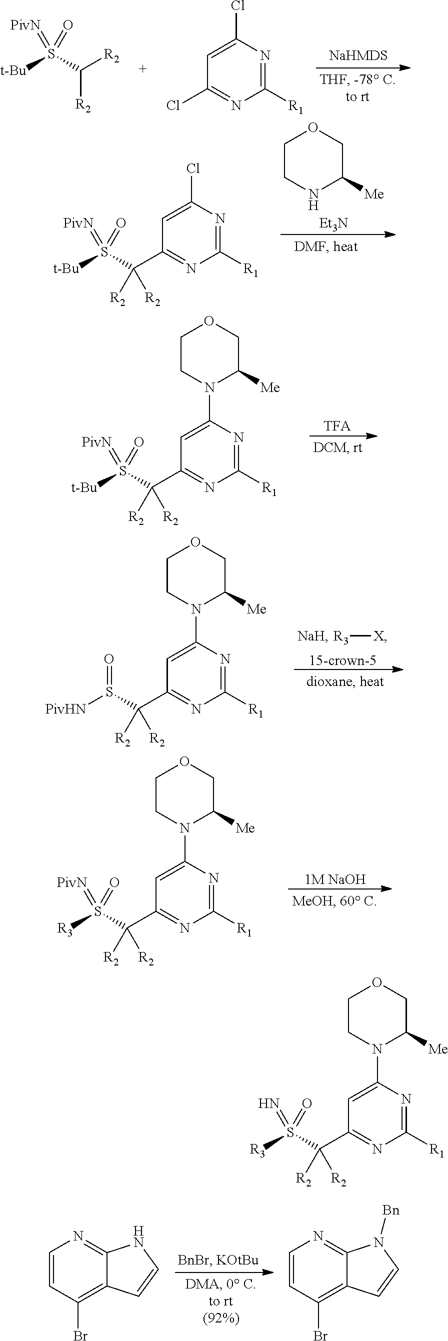

In a septum capped 100 mL round-bottomed flask equipped with a stir bar and argon balloon was added 4-bromo-1H-pyrrolo[2,3-b]pyridine (4.37 g, 22.2 mmol, 1 eq.) and DMA (26.4 mL) then cooled to 0° C. KOtBu (2.74 g, 24.4 mmol, 1.1 eq.) was added and the reaction stirred at 0° C. for 15 minutes followed by the drop-wise addition of BnBr (2.90 mL, 24.4 mmol, 1.1 eq.). The resulting reaction mixture stirred at 0° C. for one hour then warmed to room temperature where it stirred for 12 hours. The reaction mixture was diluted with water (50 mL) and EtOAc (75 mL). The pH was adjusted to pH of 3 using 2M HCl then further diluted with EtOAc (300 mL). The organic layer was washed with water (75 mL) then brine (75 mL) and dried over Na₂SO₄, filtered and concentrated to give a crude oil. Further purification by silica gel column chromatography using hexanes/EtOAc (0% to 10% EtOAc) provided 1-benzyl-4-bromo-1H-pyrrolo[2,3-b]pyridine (5.84 g, 20.3 mmol, 92% yield) as a clear colorless oil that darkens upon standing.

TLC: R_{f}=0.56 (20% EtOAc in hexanes)

¹H NMR: (500 MHz, CDCl₃) δ 8.15 (d, J=5.2 Hz, 1H), 7.34-7.26 (m, 4H), 7.23 (d, J=3.6 Hz, 1H), 7.22-7.19 (m, 2H), 6.53 (d, J=3.5 Hz, 1H), 5.49 (s, 2H) ppm.

¹³C NMR: (126 MHz, CDCl₃) δ 147.55, 143.16, 137.28, 128.79, 128.50, 127.80, 127.48, 125.21, 121.99, 119.12, 100.33, 48.31, 15.71 ppm.

HRMS: Calc'd for C₁₄H₁₂BrN₂ [M+H⁺] 287.0178; found: 287.0180.

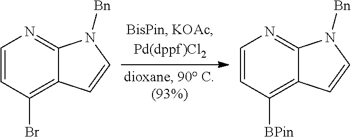

In a septum capped 250 mL round-bottomed flask equipped with a stir bar and argon balloon were added 1-benzyl-4-bromo-1H-pyrrolo[2,3-b]pyridine (5.75 g, 20.0 mmol, 1 eq.), BisPin (7.12 g, 28.0 mmol, 1.4 eq.), KOAc (3.93 g, 40.1 mmol, 2 eq.), Pd(dppf)Cl₂ (0.732 g, 1.0 mmol, 0.05 eq.) and dioxane (160 mL). The reaction mixture was degassed by bubbling argon through the solution for 5 minutes. The reaction mixture was then heated to 95° C. for 6 hours. The reaction was cooled to room temperature, diluted with EtOAc (70 mL) and filtered through a plug of Celite in a sintered glass funnel while rinsing with EtOAc (50 mL). The resulting filtrate was concentrated under reduced pressure to give a crude oil that was adsorbed to silica gel. Further purification by silica gel column chromatography using hexanes/EtOAc (0% to 10% EtOAc) provided 1-benzyl-4-(4,4,5,5-tetramethyl-1,3,2-dioxaborolan-2-yl)-1H-pyrrolo[2,3-b]pyridine (4.41 g, 13.2 mmol, 92% yield) as a light-yellow oil.

*1-benzyl-4-(4,4,5,5-tetramethyl-1,3,2-dioxaborolan-2-yl)-1H-pyrrolo [2,3-b]pyridine decomposes on LCMS to the boronic acid.

TLC: R_{f}=0.48 (20% EtOAc in hexanes, decomposes to boronic acid on TLC).

¹H NMR: (500 MHz, CDCl₃) δ 8.36 (d, J=4.6 Hz, 1H), 7.46 (d, J=4.7 Hz, 1H), 7.29-7.21 (m, 4H), 7.18-7.14 (m, 2H), 6.89 (d, J=3.5 Hz, 1H), 5.51 (s, 2H), 1.39 (s, 12H) ppm.

¹³C NMR: (126 MHz, CDCl₃) δ 147.21, 142.21, 137.96, 128.68, 128.65, 128.50, 127.50, 127.33, 124.90, 121.80, 102.01, 84.06, 47.75, 25.01 ppm.

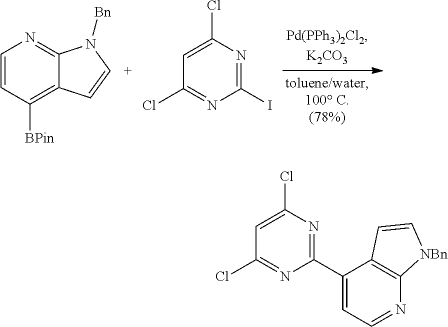

In a 250 mL round-bottomed flask equipped with a stir bar and argon balloon was added 4,6-dichloro-2-iodopyrimidine (6.15 g, 18.4 mmol, 1 eq.), 1-benzyl-4-(4,4,5,5-tetramethyl-1,3,2-dioxaborolan-2-yl)-1H-pyrrolo[2,3-b]pyridine (5.06 g, 18.4 mmol, 1 eq.), K₂CO₃ (5.09 g, 36.8 mmol, 2 eq.), Pd(PPh₃)₂Cl₂ (0.839 g, 1.2 mmol, 0.07 eq.) and toluene/H₂O (184 mL, 1.8 mL, 100:1). The reaction mixture was degassed by bubbling argon through the mixture for 30 minutes then heated to 100° C. for 18 hours. An additional 0.03 eq. of Pd(PPh₃)₂Cl₂ (0.387 g, 0.552 mmol) was added and continued heating at 100° C. for 20 hours. The reaction was cooled to room temperature, saturated aqueous NH₄Cl (200 mL) was added, aqueous layer extracted with EtOAc (4×200 mL), combined organic layers washed with brine (150 mL), dried over Na₂SO₄, filtered and concentrated. Further purification by silica gel column chromatography using hexanes/DCM (0% to 60% DCM) provided 1-benzyl-4-(4,6-dichloropyrimidin-2-yl)-1H-pyrrolo[2,3-b]pyridine (5.10 g, 14.4 mmol, 78% yield) as a yellow solid.

TLC: R_{f}=0.46 (100% DCM)

¹H NMR: (500 MHz, CDCl₃) δ 8.50 (d, J=5.0 Hz, 1H), 8.15 (d, J=5.1 Hz, 1H), 7.44 (d, J=3.5 Hz, 1H), 7.38 (d, J=3.5 Hz, 1H), 7.34 (s, 1H), 7.33-7.24 (m, 3H), 7.23-7.20 (m, 2H), 5.58 (s, 2H) ppm.

¹³C NMR: (126 MHz, CDCl₃) δ 165.61, 161.95, 149.46, 142.85, 137.55, 133.78, 130.33, 128.74, 127.70, 127.42, 119.38, 118.78, 115.93, 102.11, 48.11 ppm.

HRMS: Calc'd for C₁₈H₁₃Cl₂N₄[M+H⁺] 355.0512; found: 355.0507.

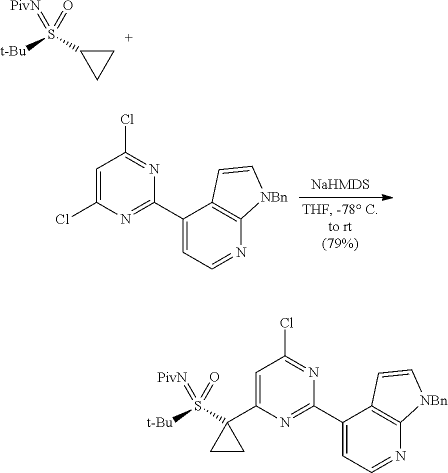

In a 250 mL round-bottomed flask equipped with a stir bar and argon balloon was added (S)—N-(tert-butyl(cyclopropyl)(oxo)-λ⁶-sulfaneylidene)pivalamide (2.35 g, 9.58 mmol, 1 eq.) and 1-benzyl-4-(4,6-dichloropyrimidin-2-yl)-1H-pyrrolo[2,3-b]pyridine (3.40 g, 9.58 mmol, 1 eq.) and THF (96 mL) then cooled to −78° C. NaHMDS (4.48 mL, 8.97 mmol, 2.2 eq, 2M in THF) was added dropwise and the reaction stirred at −78° C. for 7 hours before gradually warming to room temperature over 1 hour. Once at room temperature the reaction was quenched with saturated aqueous NH₄Cl (100 mL), aqueous layer was extracted with EtOAc (4×90 mL), combined organic layers were dried over Na₂SO₄, filtered and adsorbed to silica gel. Purification by silica gel column chromatography using hexanes/EtOAc (0% to 20% EtOAc gradient) provided (R)—N-((1-(2-(1-benzyl-1H-pyrrolo[2,3-b]pyridin-4-yl)-6-chloropyrimidin-4-yl)cyclopropyl)(tert-butyl)(oxo)-λ⁶-sulfaneylidene)pivalamide (4.28 g, 7.59 mmol, 79% yield) as a yellow oil.

TLC: R_{f}=0.19 (20% EtOAc in hexanes, UV)

¹H NMR: (500 MHz, CDCl₃) δ 8.51 (d, J=5.1 Hz, 1H), 8.17 (d, J=5.0 Hz, 1H), 7.71 (s, 1H), 7.44 (d, J=3.5 Hz, 1H), 7.38 (d, J=3.5 Hz, 1H), 7.34-7.26 (m, 3H), 7.27-7.21 (m, 4H), 5.58 (d, J=3.8 Hz, 2H), 2.58 (ddd, J=10.5, 8.1, 5.9 Hz, 1H), 2.20-2.12 (m, 1H), 1.99 (ddd, J=10.5, 7.9, 5.5 Hz, 1H), 1.47-1.40 (m, 10H), 1.27 (s, 9H) ppm.

¹³C NMR: (126 MHz, CDCl₃) δ 188.03, 165.87, 164.87, 162.03, 149.36, 142.79, 137.45, 134.68, 130.19, 128.77, 127.76, 127.51, 122.69, 118.79, 115.68, 102.12, 66.73, 48.20, 42.32, 42.28, 27.81, 24.66, 16.64, 14.62 ppm.

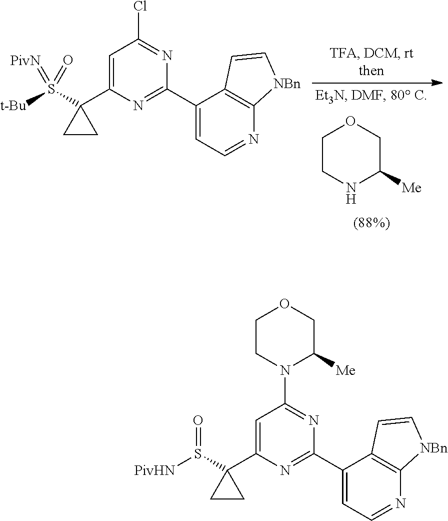

In a 200 mL round-bottomed flask equipped with a stir bar and argon balloon was added (R)—N-((1-(2-(1-benzyl-1H-pyrrolo[2,3-b]pyridin-4-yl)-6-chloropyrimidin-4-yl)cyclopropyl)(tert-butyl)(oxo)-λ⁶-sulfaneylidene)pivalamide (3.80 g, 6.74 mmol, 1 eq.) and DCM (67 mL). TFA (0.773 mL, 10.1 mmol, 1.5 eq.) was added dropwise then stirred at room temperature for 3.5 hours. The solvent was removed under reduced pressure and DMF (50 mL) was added. Et₃N (1.88 mL, 13.5 mmol, 2 eq.) and (R)-3-methylmorpholine (0.818 g, 8.09 mmol, 1.2 eq.) were added then heated to 80° C. for 68 hours. The reaction mixture was cooled to room temperature and the solvent was removed under reduced pressure to give a crude oil that was taken up in EtOAc (200 mL) and saturated aqueous NH₄Cl (200 mL). Aqueous layer extracted with EtOAc (3×200 mL), combined organic layers washed with brine (150 mL), dried over Na₂SO₄, filtered and concentrated. Further purification by silica gel column chromatography using hexanes/EtOAc (10% to 60%) provided N—((R)-(1-(2-(1-benzyl-1H-pyrrolo[2,3-b]pyridin-4-yl)-6-((R)-3-methylmorpholino)pyrimidin-4-yl)cyclopropyl)sulfinyl)pivalamide (3.42 g, 5.97 mmol, 88% yield) as a light-yellow oil.

TLC: R_{f}=0.25 (60% EtOAc in hexanes, UV)

¹H NMR: (500 MHz, CDCl₃) δ 10.10 (s, 1H), 8.45 (d, J=5.0 Hz, 1H), 7.94 (d, J=5.0 Hz, 1H), 7.29 (ddd, J=12.0, 4.5, 2.5 Hz, 4H), 7.23-7.18 (m, 3H), 6.57 (s, 1H), 5.63 (d, J=15.4 Hz, 1H), 5.51 (d, J=15.4 Hz, 1H), 4.53 (s, 1H), 4.18 (s, 1H), 4.05 (dd, J=11.5, 3.7 Hz, 1H), 3.85 (d, J=11.5 Hz, 1H), 3.75 (dd, J=11.5, 3.1 Hz, 1H), 3.59 (td, J=12.0, 3.0 Hz, 1H), 3.39 (td, J=12.8, 3.9 Hz, 1H), 1.76-1.69 (m, 1H), 1.62-1.56 (m, 1H), 1.45-1.36 (m, 5H), 0.91 (s, 9H) ppm.

¹³C NMR: (126 MHz, CDCl₃) δ 179.41, 164.08, 162.21, 161.40, 149.25, 142.83, 137.78, 137.60, 129.28, 128.71, 127.68, 127.40, 118.77, 115.80, 103.35, 101.83, 70.94, 66.72, 48.06, 47.35, 44.86, 39.39, 26.89, 13.92, 10.43, 10.20 ppm.

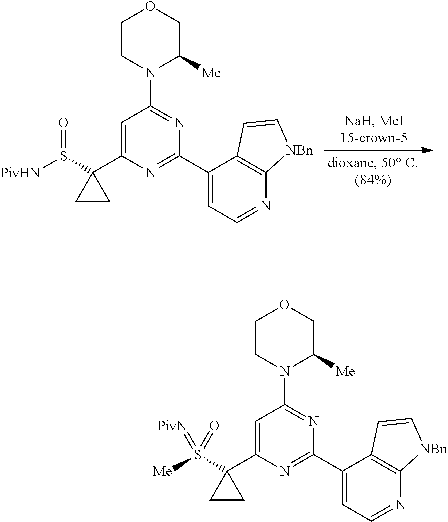

In a 20 mL septum capped vial equipped with a stir bar and argon balloon was added N—((R)-(1-(2-(1-benzyl-1H-pyrrolo [2,3-b]pyridin-4-yl)-6-((R)-3-methylmorpholino)pyrimidin-4-yl)cyclopropyl)sulfinyl)pivalamide (0.375 g, 0.654 mmol, 1 eq.) and dioxane (6.5 mL). 15-crown-5 ether (0.155 mL, 0.785, 1.2 eq.) was added followed by NaH (0.031 g, 0.785 mmol, 60% wt, 1.2 eq.) then stirred at room temperature for 25 minutes. Mel (0.082 mL, 1.31 mmol, 2 eq.) was added and the reaction mixture was heated to 50° C. for 20 hours. The reaction was cooled to room temperature, quenched with saturated aqueous NH₄Cl (15 mL), aqueous layer extracted with DCM (4×20 mL), combined organic layers washed with brine (20 mL), dried over Na₂SO₄, filtered and concentrated. Further purification by silica gel column chromatography using hexanes/EtOAc (0% to 40%) provided N—((R)-(1-(2-(1-benzyl-1H-pyrrolo[2,3-b]pyridin-4-yl)-6-((R)-3-methylmorpholino)pyrimidin-4-yl)cyclopropyl)(methyl)(oxo)-λ⁶-sulfaneylidene)pivalamide (0.322 g, 0.549 mmol, 84% yield) as a light-yellow oil.

TLC: R_{f}=0.57 (60% EtOAc in hexanes, UV)

¹H NMR: (500 MHz, CDCl₃) δ 8.47 (d, J=5.0 Hz, 1H), 8.04 (d, J=5.0 Hz, 1H), 7.32-7.26 (m, 5H), 7.24-7.21 (m, 2H), 6.78 (s, 1H), 5.57 (s, 2H), 4.67-4.57 (m, 1H), 4.12-3.99 (m, 2H), 3.84 (d, J=11.5 Hz, 1H), 3.73 (dd, J=11.5, 3.0 Hz, 1H), 3.57 (td, J=11.9, 3.1 Hz, 1H), 3.45 (s, 3H), 3.44-3.35 (m, 1H), 2.18 (ddd, J=10.4, 7.5, 5.8 Hz, 1H), 1.89 (ddd, J=10.5, 7.3, 5.2 Hz, 1H), 1.70 (ddd, J=9.4, 7.2, 5.8 Hz, 1H), 1.53-1.47 (m, 1H), 1.39 (d, J=6.8 Hz, 3H), 1.07 (s, 9H) ppm.

¹³C NMR: (126 MHz, CDCl₃) δ 188.06, 163.70, 162.12, 161.29, 142.71, 137.66, 129.24, 128.73, 127.68, 127.50, 118.85, 115.59, 103.46, 101.91, 71.01, 66.71, 48.13, 47.13, 46.54, 41.35, 40.03, 39.54, 27.64, 13.95, 13.10, 12.72 ppm.

Exemplary synthetic schemes of specific compounds are also provide below.

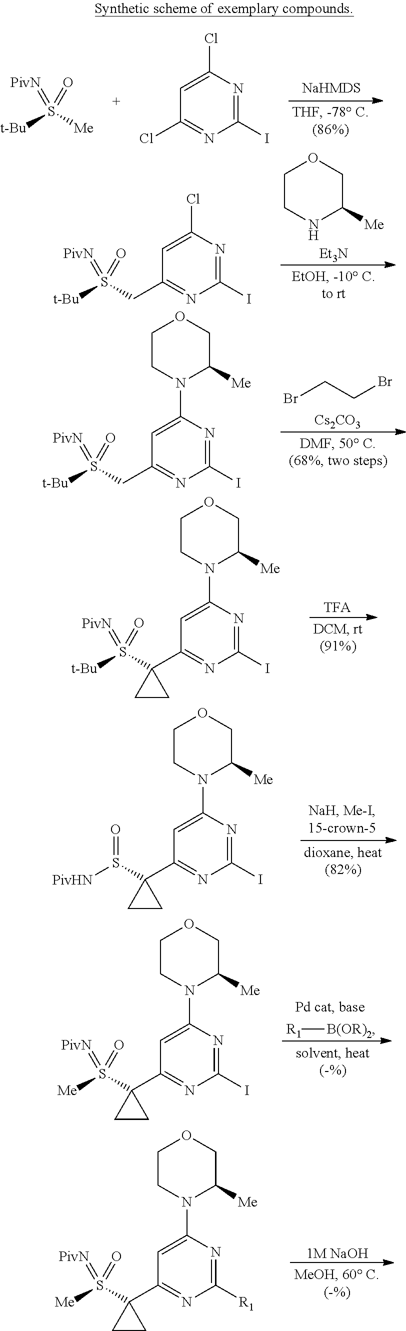

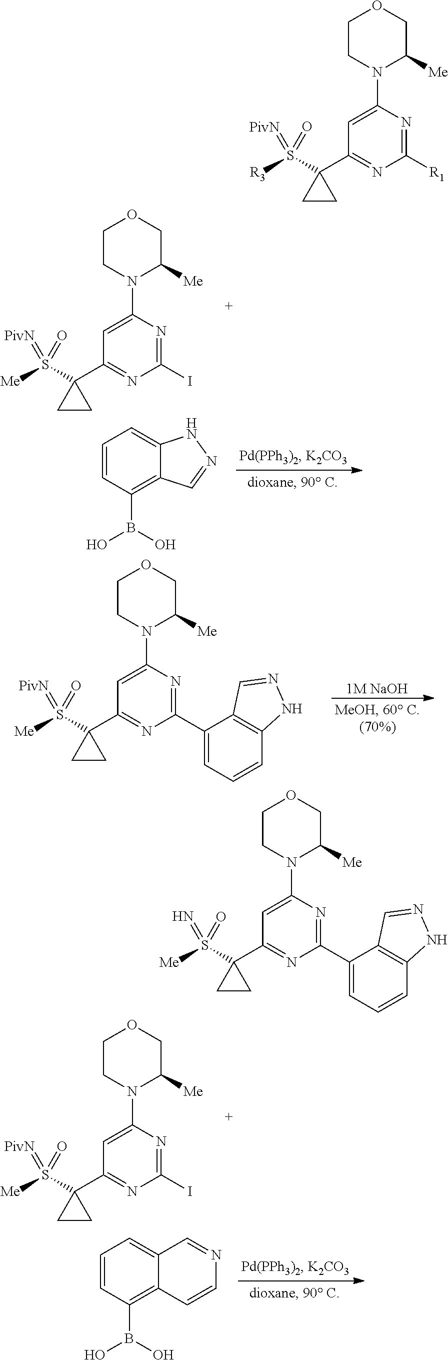

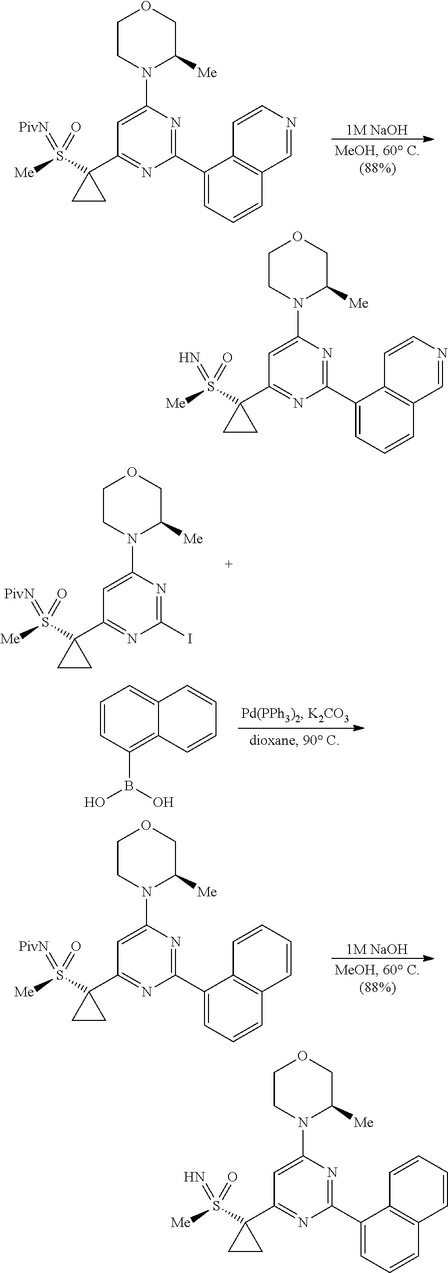

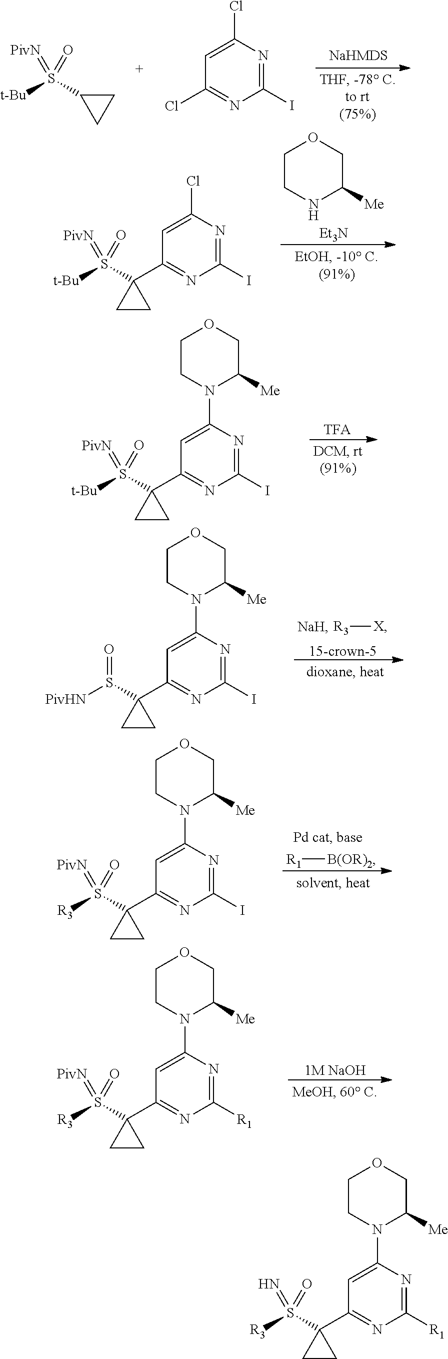

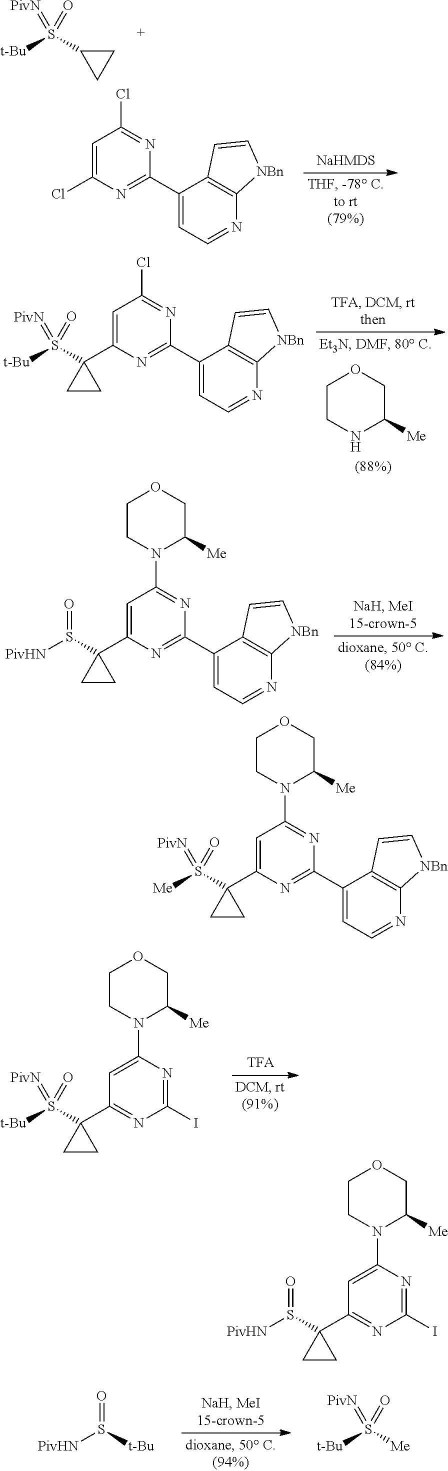

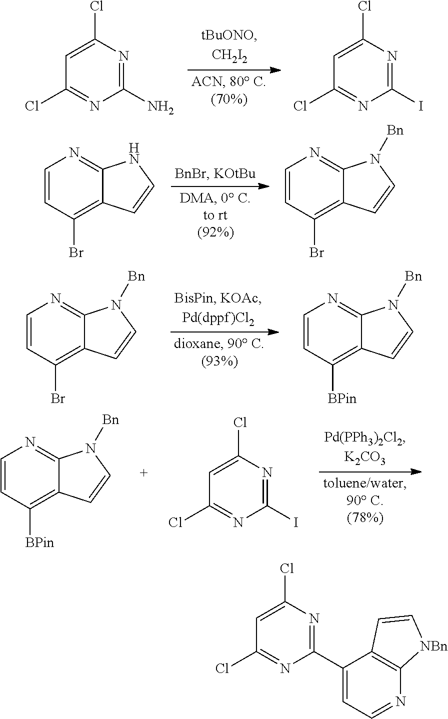

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A compound having Formula I′. 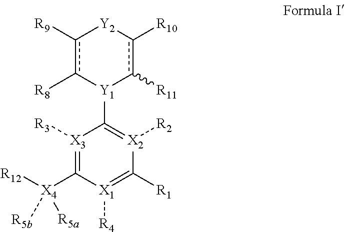
wherein
X₁, X₂, and X₃ are independently selected from C, N, or S;
X₄ is selected from C, N, or S;
Y₁ is selected from CH or N;
Y₂ is selected from O or NH;
R₁ is selected from halogen, amine, alkylamine, thiol, sulfide, sulfonyl, sulfoximine, sulfoxide, sulfur diimide, sulphonamide, alkoxyl, cycloalkyl, heterocycloalkyl, aryl, or heteroaryl, wherein R₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₂, R₃, and R₄, when present, are independently selected from, hydrogen, halogen, hydroxyl, cyano, carboxyl, C₁-C₃ alkyl, C₁-C₃ haloalkyl, or cyclopropyl;
R₅ₐ and R_{5b}, when present, are independently selected from, hydrogen, halogen, hydroxyl, cyano, carboxyl, amino, aminoalkyl, C₁-C₆ alkyl, C₁-C₆ haloalkyl, cycloalkyl, heterocycloalkyl, or R₅ₐ and R_{5b} combine to form a carbonyl, a C₃-C₈ cycloalkyl or a C₂-C₇ heterocycloalkyl together with the atom to which they are attached, wherein R₅ₐ and R_{5b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₈ is selected from hydrogen, halogen, hydroxyl, cyano, carboxyl, amino, C₁-C₃ alkyl, C₁-C₃ haloalkyl, or C₁-C₃ aminoalkyl, wherein R₈ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₉ and R₁₀ are independently selected from hydrogen, halogen, hydroxyl, cyano, carboxyl, amino, C₁-C₃ alkyl, C₁-C₃ haloalkyl, or C₁-C₃ aminoalkyl, wherein R₉ and R₁₀ are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₁ is selected from C₁-C₃ alkyl, C₁-C₃haloalkyl, or C₁-C₃ aminoalkyl, wherein R₁₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₁₂ is selected from sulfoximine, sulfonyl, sulfoxide, sulfur diimide, sulphonamide, amide, amine, hydroxyl, carbonyl, ester, alkyl, heteroalkyl, or one of R₅ₐ or R_{5b} combine with R₁₂ to form a C₃-C₈ cycloalkyl or a C₂-C₇ heterocycloalkyl together with the atom to which they are attached, wherein R₁₂ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl; and [symbol]
represents a bond that is present or absent,
or a pharmaceutically acceptable salt thereof, and
wherein the compound is not AZD6738, AZ20, or AZD3147.

2-3. (canceled)

4. The compound of claim 1, having the formula of Formula II. 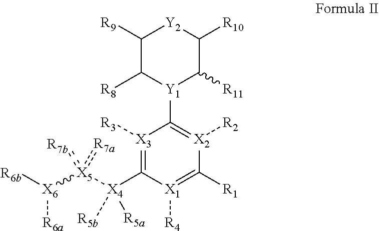
wherein
R₆ₐ and R_{6b} are independently selected from, hydrogen, halogen, hydroxyl, cyano, carboxyl, amino, aminoalkyl, C₁-C₆ alkyl, C₁-C₆ haloalkyl, cycloalkyl, heterocycloalkyl, aryl, heteroaryl, or R₆ₐ and R_{6b} combine to form a C₃-C₈ cycloalkyl, a C₂-C₇ heterocycloalkyl, an aryl, or a heteroaryl together with the atom to which they are attached, wherein R₆ₐ and R_{6b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl; or
wherein two or more of R₅ₐ, R_{5b}, R₆ₐ, and R_{6b} combine together with the atom to which they are attached to form a C₂-C₇ heterocycloalkyl or C₂-C₇ heterocycloalkenyl;
R₇ₐ and R_{7b} are independently absent or present and when present, are selected from O or NH;
or a pharmaceutically acceptable salt thereof.

5. The compound of claim 1, having a structure according to Formula II-A. 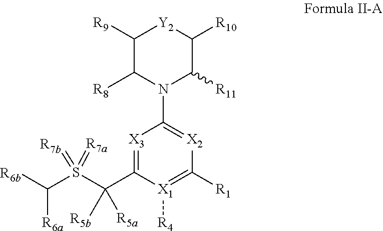
wherein
X₁, X₂, and X₃ are independently selected from C, CH, or N;
Y₂ is selected from O or NH;
R₁ is selected from heterocycloalkyl, aryl or heteroaryl, wherein R₁ is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₄, when present, is selected from, hydrogen, halogen, hydroxyl, cyano, carboxyl, C₁-C₃ alkyl, C₁-C₃ haloalkyl, or cyclopropyl;
R₅ₐ and R_{5b} are independently selected from, C₁-C₃ alkyl, C₁-C₃ haloalkyl, C₃-C₅ cycloalkyl, C₁-C₃ heterocycloalkyl, or R₅ₐ and R_{5b} combine to form a C₃-C₈ cycloalkyl or a C₂-C₇ heterocycloalkyl together with the atom to which they are attached, wherein R₅ₐ and R_{5b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₆ₐ and R_{6b} are independently selected from, hydrogen, halogen, hydroxyl, cyano, amino, aminoalkyl, C₁-C₆ alkyl, C₁-C₆ haloalkyl, C₃-C₆ cycloalkyl, heterocycloalkyl, aryl, heteroaryl, or R₆ₐ and R_{6b} combine to form a C₃-C₈ cycloalkyl, a C₂-C₇ heterocycloalkyl, an aryl, or a heteroaryl together with the atom to which they are attached, wherein R₆ₐ and R_{6b} are independently and optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl;
R₇ₐ and R_{7b} are independently selected from 0 or NH;
R₈, R₉, and R₁₀ are independently selected from hydrogen, halogen, hydroxyl, cyano, amino, C₁-C₃ alkyl, C₁-C₃ haloalkyl, or C₁-C₃ aminoalkyl;
R₁₁ is selected from C₁-C₃ alkyl, C₁-C₃haloalkyl, or C₁-C₃ aminoalkyl; and [symbol]
represents a bond that is present or absent,
or a pharmaceutically acceptable salt thereof.

6. The compound of claim 1, wherein R¹ is selected from thiol, sulfide, sulfonyl, halogen, amine, alkoxyl, heterocycloalkyl, C₅-C₁₀ aryl, or C₂-C₉ heteroaryl, wherein R₁ is optionally substituted with alkyl, halogen, amine, hydroxyl, thiol, sulfide, or sulfonyl.

7-9. (canceled)

10. The compound of claim 1, wherein R¹ is methyl sulfide.

11. (canceled)

12. The compound of claim 1, wherein X₁ and X₂ are both N.

13. The compound of claim 1, wherein Y₁ is N and Y₂ is O.

14-15. (canceled)

16. The compound of claim 1, wherein X₃ is C and R₃ is present and selected from hydrogen or C₁-C₃ alkyl.

17-18. (canceled)

19. The compound of claim 4, wherein X₅ is S.

21-22. (canceled)

23. The compound of claim 1, wherein
R₅ₐ and R_{5b} combine to form a carbonyl, a C₃-C₈ cycloalkyl or a C₂-C₄ heterocycloalkyl together with the atom to which they are attached.

24. (canceled)

25. The compound of claim 1, wherein R₅ₐ and R_{5b} combine to form a C₃ cycloalkyl or together with the atom to which they are attached.

26-31. (canceled)

32. The compound of claim 1, wherein R₈, R₉, and R₁₀ are hydrogen or substituted- or unsubstituted C₁-C₃ alkyl.

33. The compound of claim 1, wherein R₈, R₉, and R₁₀ are hydrogen.

34. The compound of claim 1, wherein R₁₁ is selected from a substituted or unsubstituted C₁-C₃ alkyl.

35. The compound of claim 1, having a structure represented by a formula: 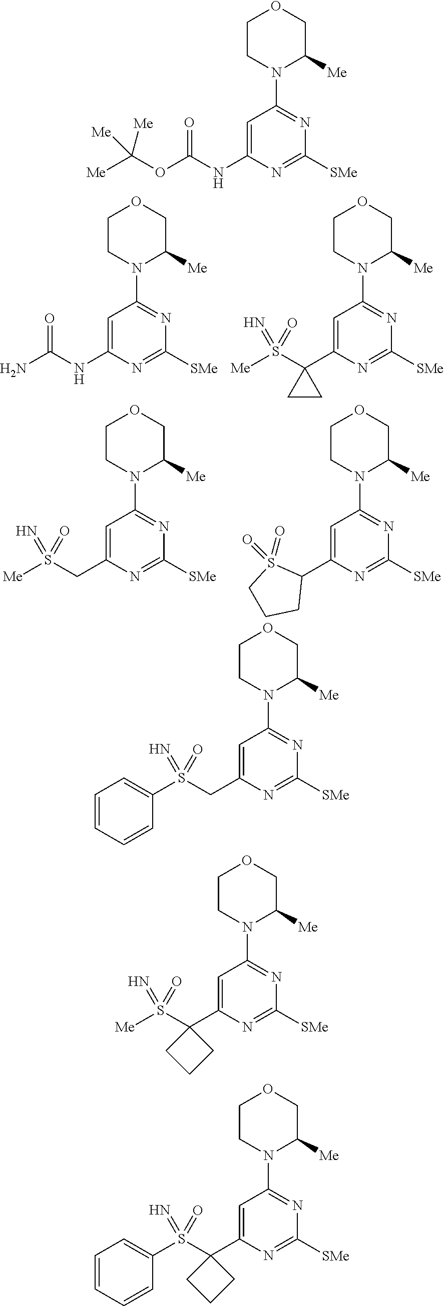 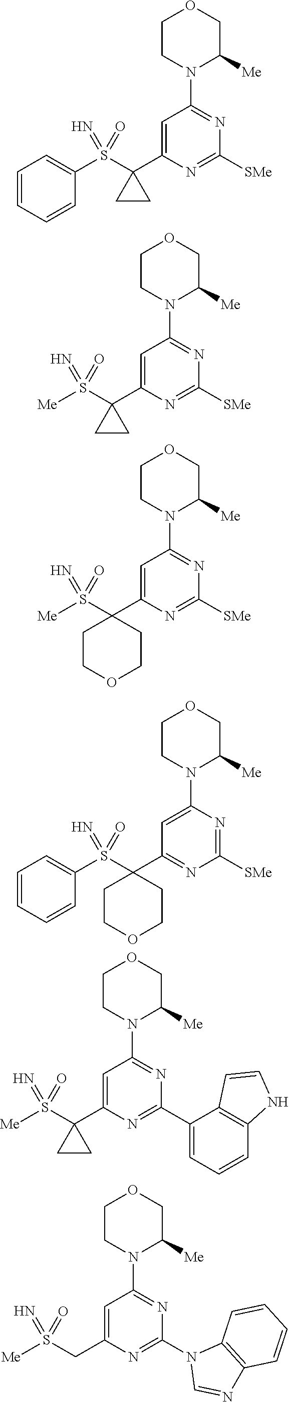 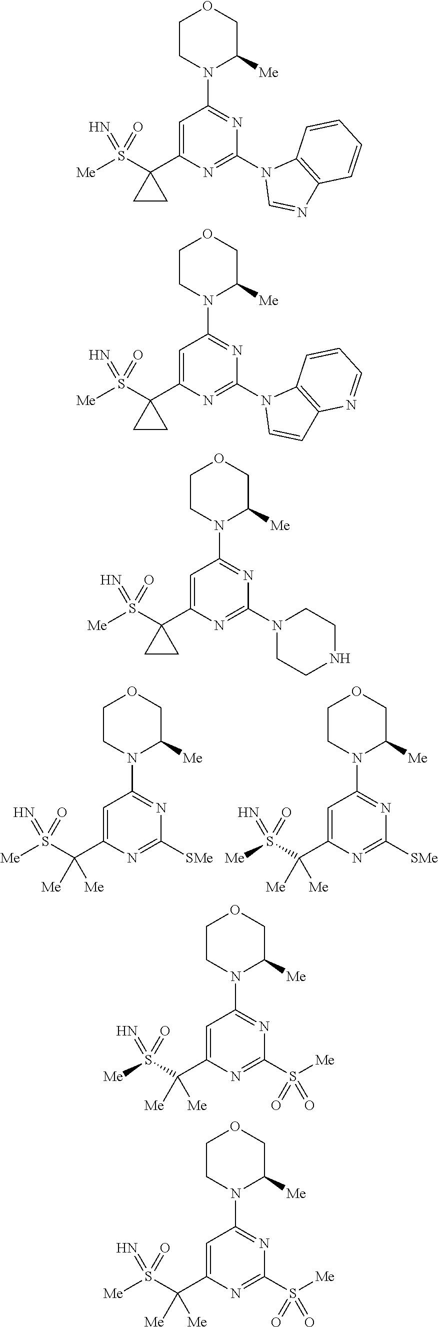 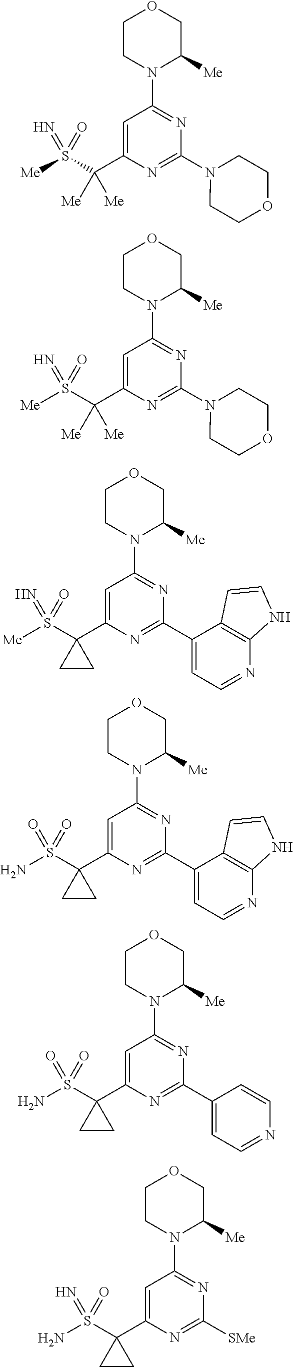 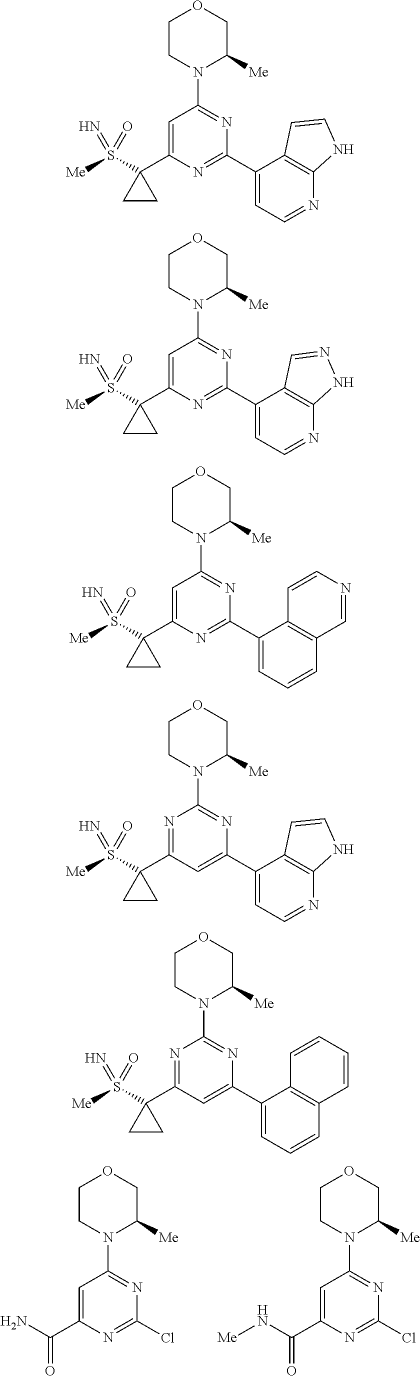 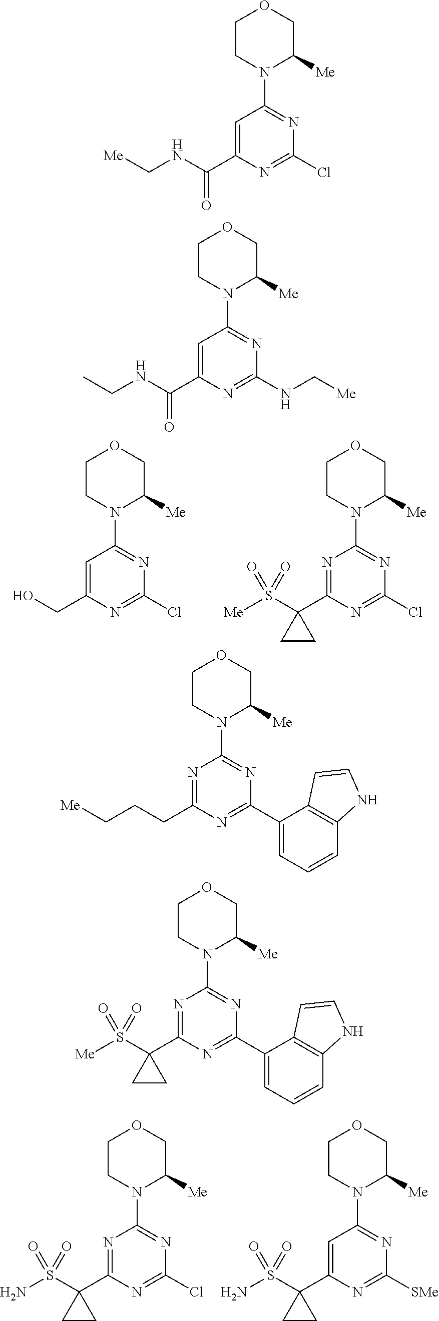 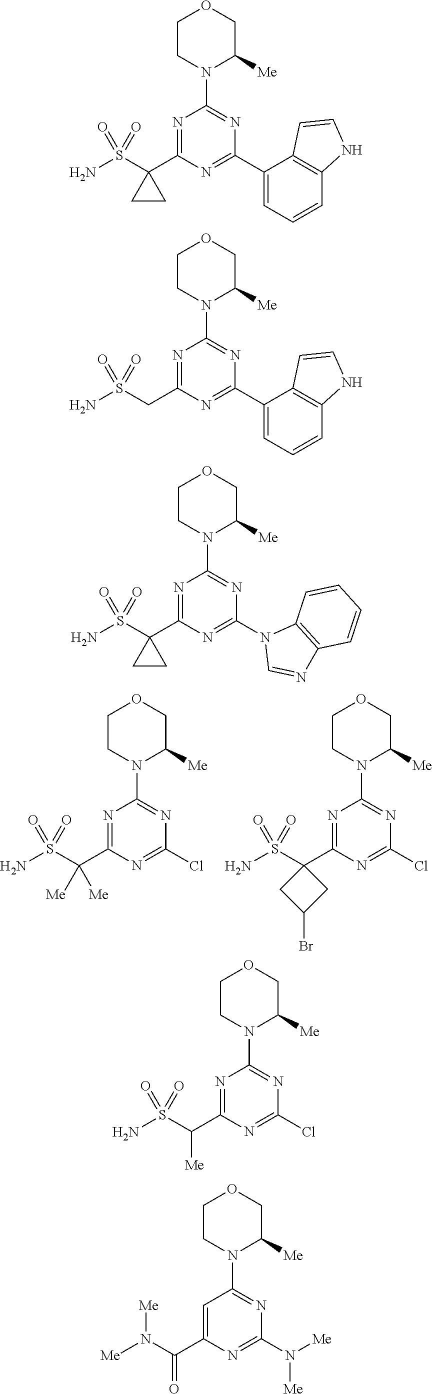 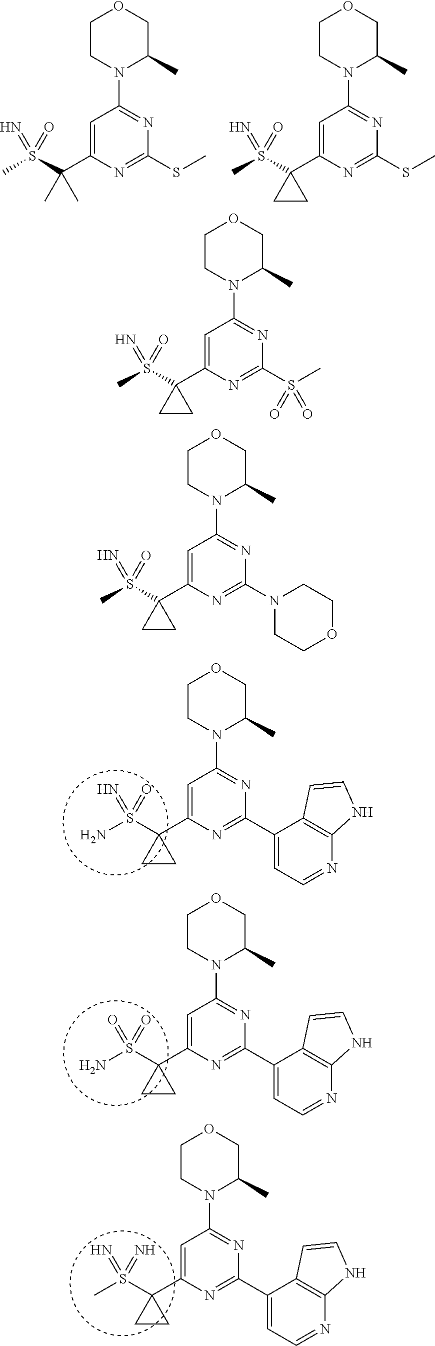 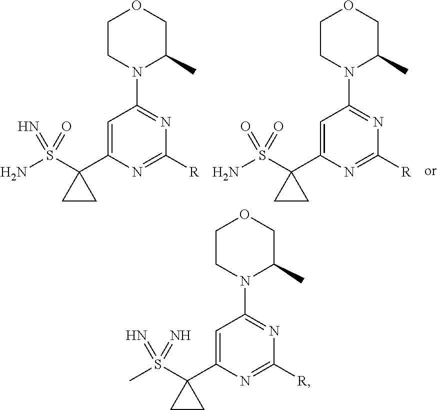
wherein R is selected from halogen, amine, alkylamine, thiol, sulfide, sulfonyl, sulfoximine, sulfoxide, sulfur diimide, sulphonamide, alkoxyl, cycloalkyl, heterocycloalkyl, aryl, or heteroaryl, wherein R is optionally substituted with alkyl, haloalkyl, alkoxyl, halogen, amine, amide, nitro, cyano, hydroxyl, thiol, sulfide, sulfonyl, sulfoximine, sulfoxide, sulfur diimide, sulphonamide, aryl, or heteroaryl.

36. (canceled)

37. A pharmaceutical composition comprising a therapeutically effective amount of the compound of claim 1, or a pharmaceutically acceptable salt thereof, and a pharmaceutically acceptable carrier.

38. A method for the treatment of a disorder of uncontrolled cellular proliferation associated with TAF1 dysfunction in a mammal comprising the step of administering to the mammal an effective amount of the compound according to claim 1.

39-40. (canceled)

41. The method of claim 38, wherein the mammal is human; and wherein the human has been identified to have Mdm2-mediated degradation of the tumor suppressor p53.

42-43. (canceled)

44. The method of claim 38.

45. The method of claim 44, wherein the cancer is selected from breast cancer, lung cancer, cervical cancer, gastrointestinal cancer, colorectal cancer, brain cancer, skin cancer, prostate cancer, ovarian cancer, thyroid cancer, testicular cancer, pancreatic cancer, endometrial cancer, melanoma, glioma, leukemia, lymphoma, chronic myeloproliferative disorder, myelodysplastic syndrome, myeloproliferative neoplasm, and plasma cell neoplasm (myeloma).

46-56. (canceled)
